(19) United States
(12) Patent Application Publication
(10) Pub. No.: US 2008/0051454 A1
(43) Pub. Date: Feb. 28, 2008

(54) Compositions and methods for transient receptor potential vanilloid (TRPV) channel mediated treatments

(75) Inventor: Donna H. Wang, Okemos, MI (US)

Correspondence Address: MEDLEN & CARROLL, LLP, 101 HOWARD STREET, SUITE 350, SAN FRANCISCO, CA, 94105, US

(73) Assignee: The Board of Trustees Operating Michigan State University, East Lansing, MI (US)

(21) Appl. No.: 11/788,877

(22) Filed: Apr. 23, 2007

Related U.S. Application Data
(60) Provisional application No. 60/794,019, filed on Apr. 21, 2006.

(51) Int. Cl. A61K 31/20; A61K 31/164; A61K 31/165; C12Q 1/02; A61P 9/00; A61K 31/19

(52) U.S. Cl. 514/559; 435/29; 514/557; 514/622; 514/627

(57) ABSTRACT

The present inventions relate to therapeutic compositions comprising, and methods utilizing, arachidonic acid derivatives and analogs for treatment of patients demonstrating symptoms of pathological conditions. Specifically, the inventions relate to therapeutic compositions for activating transient receptor potential vanilloid-1 channels (TRPV1). Additionally, therapeutic compositions are provided for increasing TRPV1-type responses. These pathological conditions include, but are limited to, hypertension, in particular salt induced hypertension, and cardiovascular complications, including myocardial infarction, kidney dysfunction, diabetes, and inflammation. Further, the inventions relate to drug screening methods for providing additional therapeutic compounds.

The present invention claims priority to U.S. Provisional Patent Application Ser. No. 60/794,019 filed Apr. 21, 2006, hereby incorporated by reference in its entirety.

This invention was made in part with government support under grants NIH 71-3010, HL-57853, HL-73287, and DK67620 awarded by the United States National Institutes of Health. The government has certain rights in the invention.

### FIELD OF THE INVENTION

The present inventions relate to therapeutic compositions comprising, and methods utilizing, arachidonic acid derivatives and analogs for treatment of patients demonstrating symptoms of pathological conditions. Specifically, the inventions relate to therapeutic compositions for activating transient receptor potential vanilloid-1 channels (TRPV1). Additionally, therapeutic compositions are provided for increasing TRPV1-responses. These pathological conditions include, but are limited to, hypertension, in particular salt induced hypertension, and cardiovascular complications, including myocardial infarction, kidney dysfunction, diabetes, and inflammation. Further, the inventions relate to drug screening methods for providing additional therapeutic compounds.

### BACKGROUND

There are an estimated that 65 million individuals in the United States alone suffering from hypertension including a high number of patients with salt sensitive hypertension. Yet there are few if any effective treatments for salt sensitive hypertension. Current treatments for hypertension are disruptive to the patient and not well tolerated. The detrimental consequences of this disease involve myocardial infarction, congestive heart failure, stroke, and renal failure.

Despite intensive research in this field, the molecular basis underlying human hypertension is essentially largely unknown with the exception of the involvement of the sensory nervous system. Thus pharmacologic prevention of end organ damage induced by hypertension remains a challenge. Defining how sensory nerves sense changes in the environment, alter their afferent and efferent activities, and cross-talk with other systems to modulate cardiovascular and renal function and blood pressure may provide valuable new insight into the interactions that lead to hypertension and increased salt sensitivity. In particular, defining specific receptors or receptor systems of sensory nerves should provided new areas for targeted drug therapies. Such insight may unveil novel pharmacologic approaches in order to reduce hypertension and prevent the resulting end organ damage.

What is needed, therefore, are compositions and methods for the treatment of hypertension, in particular salt sensitive hypertension, and cardiovascular disease that are more effective and better tolerated than current treatment modalities.

### SUMMARY OF THE INVENTION

The present inventions relate to therapeutic compositions comprising, and methods utilizing, arachidonic acid derivatives and analogs for treatment of patients demonstrating symptoms of pathological conditions. Specifically, the inventions relate to therapeutic compositions for activating transient receptor potential vanilloid-1 channels (TRPV1). Additionally, therapeutic compositions are provided for increasing TRPV1-responses. These pathological conditions include, but are limited to, hypertension, in particular salt induced hypertension, and cardiovascular complications, including myocardial infarction, kidney dysfunction, diabetes, and inflammation. Further, the inventions relate to drug screening methods for providing additional therapeutic compounds.

Embodiments of the present invention relate to therapeutic compositions comprising, endogenous molecules, derivatives of endogenous molecules, and synthetic forms of endogenous molecules. In other embodiments, the present inventions relate to therapeutic compositions comprising novel synthetic compounds for activating TRPV1 receptors. In other embodiments, compounds of the present inventions bypass TRPV1 receptors for inducing TRPV1 activation effects. Embodiments of the present invention further relate to therapeutic compositions comprising, and methods utilizing, arachidonic acid derivatives and analogs for treating patients.

In one embodiment, transient receptor potential vanilloid-1 channel agonists are provided for treating patients at risk for, or currently experiencing, high blood pressure and/or cardiac related symptoms.

In one embodiment, the present invention relates to using a 20-hydroxyeicosatetraenoic acid (20-HETE) analog (referred to as DSR-II-247-30) having the following structure: 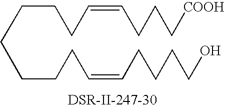
for treating hypertension patients. In a further embodiment, hypertension patients are salt sensitive hypertension patients.

The present invention is not limited a specific compound. Indeed, in some embodiments, the invention provides derivatives and analogs of arachidonic acid:

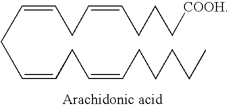

In other embodiments, the invention describes analogs of 20-HETE:

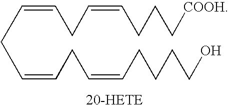

In a preferred embodiment, the invention comprises a compound having the following structure:

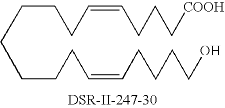

In other embodiments, the invention comprises a compound, referred to as TVR-I-80-20, which has the following structure:

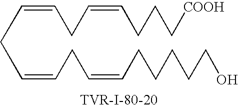

In other embodiments, the invention comprises a compound, referred to as GKD-II-56-22, which has the following structure:

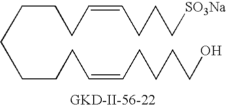

In other embodiments, the invention comprises a compound, referred to as SA-II-54-25, which has the following structure:

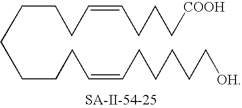

In other embodiments, the invention comprises a compound, referred to as N-oleoyl-dopamine, which has the following structure:

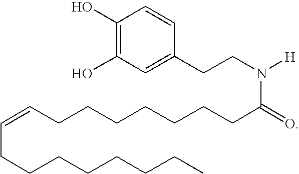

In one embodiment, the invention provides a method of treating a subject, comprising: a) providing: i) a subject, and ii) a pharmaceutical composition comprising a 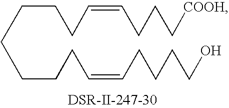
therapeutic agent, N-arachidonoyl dopamine (NADA), N-oleoyl-dopamine (OLDA), anandamide, methanandamide (MethA), 20-hydroxyeicosatetraenoic acid (20-HETE), capsaicin (CAP), derivative and synthetic analog thereof, and a pharmaceutical carrier, and b) administering said pharmaceutical composition to said subject. It is not intended that the present invention be limited by the therapeutic agent. Therapeutic agents include, but are not limited to 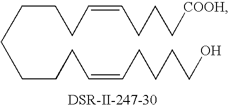
N-arachidonoyl dopamine (NADA), N-oleoyl-dopamine (OLDA), anandamide, methanandamide (MethA), 20-hydroxyeicosatetraenoic acid (20-HETE), capsaicin (CAP), derivative and synthetic analog thereof. The present invention is not limited to any particular concentration of therapeutic agent. In some embodiments, the invention provides a preparation wherein said therapeutic agent is administered at 0.5 mg/kg. In some embodiments, the invention provides a preparation wherein said therapeutic agent is administered at 1.0 mg/kg. In some embodiments, the invention provides a preparation wherein said therapeutic agent is administered at 10 mg/kg. In some embodiments, the invention provides a preparation wherein said therapeutic agent is administered at a concentration of at least, 0.5, 1.0, 2.0, 5.0, 8.0, 10.0, 50.0 and 100.00 mg/kg. The present invention is not limited to any particular type of pharmaceutical carrier. Indeed, in some embodiments, the pharmaceutical carrier includes, but is not limited to saline, a solvent, and a wetting agent. The present invention is not limited to any particular type of solvent. Indeed, in some embodiments, the solvent included but is not limited to ethanol and dimethyl sulfoxide (DMSO). The present invention is not limited to any particular type of wetting agent. Indeed, in some embodiments, the wetting agent is at least one or more of Tween 20, Tween 80 Tween 60, Tween 85, Brij 35, Brij 78, Myrj 52, PEG 600, glycerin, sodium lauryl sulfate, Pluronic F-68, Pluronic F-38, Pluronic P-105, PLURONIC L101, LURONIC L121, and Pluronic-10R5. In some embodiments, the pharmaceutical carrier is a targeting agent for sensory nerves. In some embodiments, the pharmaceutical composition further comprises a side-effect reducing agent, wherein said side-effect reducing agent is co-administered to said patient. The present invention is not limited to any particular type of side-effect reducing agent. In some embodiments, the side-effect reducing agent is a cannabinoid-1 receptor antagonist or a derivative or synthetic analog thereof. In some embodiments, the cannabinoid-1 receptor antagonist is SR141716A. The present invention is not limited to any particular type of administering a therapeutic agent. In some embodiments, the invention provides a method wherein said administering comprises intra-arterial injection. In some embodiments, the invention provides a method wherein said administering comprises intravenous injection. In some embodiments, the invention provides a method wherein said administering comprises a local injection. In some embodiments, the invention provides a method wherein said preparation further comprises a local anesthetic. The present invention is not limited to any particular subject. Indeed, the present invention contemplates a range of subjects for administering a therapeutic. In a preferred embodiment, a subject may present symptoms of a pathological condition. In some embodiments, the subject is a patient. In some embodiments, the patient is at risk for hypertension. In some embodiments, the subject presents one or more symptoms indicative of salt sensitive hypertension. In some embodiments, the invention provides a method of treatment to a subject, wherein said subject presents one or more symptoms indicative of salt sensitive hypertension. The present invention is not limited to any particular symptom of salt sensitive hypertension. In some embodiments, the invention provides a method wherein said symptom of salt sensitive hypertension comprises at least one or more of a high dietary salt intake correlating with high blood pressure, increase in high mean arteriole pressure correlating with increasing sodium intake, high blood pressure, high mean arteriole pressure, chronic hypertension, low plasma renin levels, and a decrease in sensory nerve function. In some embodiments, the invention provides a method wherein said patient is selected from a group consisting of a subject with a genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, a subject with a TRPV1 receptor, a subject with a dysfunctional and/or compromised TRPV1 receptor, and a subject with high salt intake. Subjects may present cardiac related symptoms. In some embodiments, the subject presents one or more symptoms indicative of cardiovascular disease. In particular, cardiac symptoms may be indicative of cardiac myopathy or cardiac infraction or cardiac ischemia wherein said symptoms may be pre or post event symptoms. In some embodiments, subjects may comprise healing heart tissue. In some embodiments, subjects may present renal dysfunction symptoms. In some embodiments, subjects may present symptoms of aging. In some embodiments, subjects may present symptoms of inflammation in cardiovascular tissues. In another embodiment, the present invention contemplates the use of the arachidonic acid analogs and derivatives, described in the instant application, as an analgesic.

In one embodiment, the invention provides a method of treating a patient demonstrating at least one symptom of salt sensitive hypertension, comprising: a) providing: i) a patient demonstrating one or more symptoms of salt induced hypertension, and ii) a pharmaceutical composition comprising a therapeutic agent, wherein said therapeutic agent is selected from the group consisting of 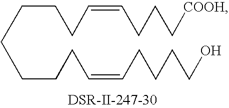
N-arachidonoyl dopamine (NADA), N-oleoyl-dopamine (OLDA), anandamide, Methanandamide (MethA), 20-hydroxyeicosatetraenoic acid (20-HETE), capsaicin (CAP), derivative or synthetic analog thereof, and a pharmaceutical carrier; and b) administering said formulation to said patient under a condition such that one or more symptom of salt induced hypertension is reduced.

In some embodiments, the invention provides a method wherein said subject is a human. In some embodiments, the invention provides a method of treating a patient demonstrating at least one symptom of salt sensitive hypertension, comprising: a) providing: i) a patient demonstrating one or more symptoms of salt induced hypertension, and ii) a formulation comprising the compound: 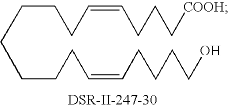
and b) administering said formulation to said patient under condition such that one or more symptom of salt induced hypertension is reduced.

In some embodiments, the invention provides a method wherein said symptom of salt sensitive hypertension comprises at least one or more of a high dietary salt intake correlating with high blood pressure, increase in high mean arteriole pressure correlating with increasing sodium intake, high blood pressure, high mean arteriole pressure, chronic hypertension, low plasma renin levels, and a decrease in sensory nerve function.

In some embodiments, the invention provides a method wherein said patient is selected from a group consisting of a subject with a genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, a subject with dysfunctional and/or compromised TRPV1 receptors, and a subject with high salt intake.

In one embodiment, the invention provides a method of treating a patient demonstrating at least one symptom of salt sensitive hypertension, comprising: a) providing: i) a patient demonstrating one or more symptoms of salt induced hypertension, and ii) a pharmaceutical composition comprising a therapeutic agent, wherein said therapeutic agent is selected from the group consisting of 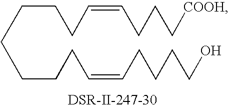
N-arachidonoyl dopamine (NADA), N-oleoyl-dopamine (OLDA), anandamide, Methanandamide (MethA), 20-hydroxyeicosatetraenoic acid (20-HETE), capsaicin (CAP), derivative or synthetic analog thereof, and a pharmaceutical carrier; and b) administering said formulation to said patient under a condition such that one or more symptom of salt induced hypertension is reduced. 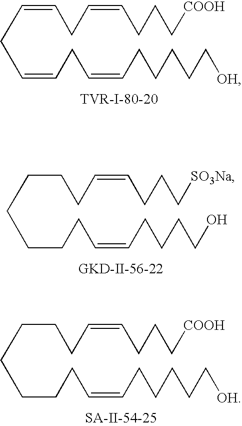
In some embodiments, said symptom of salt sensitive hypertension is selected from the group consisting of increased blood pressure, increased mean arteriole pressure, and decreased plasma renin levels. In some embodiments, said patient is selected from a group consisting of a subject with a genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, and a subject with high salt intake.

In one embodiment, the invention provides a method for drug screening comprising exposing a cell expressing a Transient Receptor Potential Vanilloid-1 (TRPV1) receptor to a test compound of interest and determining the activity of said cell in the presence and absence of said test compound.

In some embodiments, said cell is provided in a tissue. In some embodiments, the invention provides a method wherein said symptom of salt sensitive hypertension comprises at least one or more of a high dietary salt intake correlating with high blood pressure, increase in high mean arteriole pressure correlating with increasing sodium intake, high blood pressure, high mean arteriole pressure, chronic hypertension, low plasma renin levels, and a decrease in sensory nerve function.

In some embodiments, the invention provides a method wherein said patient is selected from a group consisting of a subject with a genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, a subject with dysfunctional and/or compromised TRPV1 receptors, and a subject with high salt intake.

In some embodiments, the invention provides a method wherein said patient is at risk for hypertension, wherein at risk for hypertension comprises at least one or more of a history of high blood pressure in the family, a family member who suffered from a stroke, a frequent dieter, older than age 45, a sign of inflammation in cardiovascular or renal tissues, consumes a high salt diet, children with renal dysfunction, renal deficiency, renal dysfunction, consume more than two alcoholic beverages per day, overweight, obesity, ethnic group, for example, Hispanic and African American.

In one embodiment, the invention provides a method for drug screening comprising: a) providing: i) a cell expressing a Transient Receptor Potential Vanilloid-1 (TRPV1) receptor; and ii) a test compound; b) exposing the cell to the test composition; and c) determining the activity of said cell in the presence of said test composition. In some embodiments, said test compound is provided in a pharmaceutical carrier. It is not meant to limit the type of cells. Indeed, cells include but are not limited to a neuronal cell, a cardiac cell, a kidney cell, and an engineered cell. It is not meant to limit the location of or population of cells. In some embodiments, said cell is located within a tissue. In some embodiments, said cell is in vivo. It is not meant to limit the activity of a cell. Indeed, cell activity includes but is not limited to decreasing mean arteriole pressure, protecting against ischemia and reperfusion injury, decreasing ventricular end-diastolic pressure, increasing coronary flow, increasing ventricular peak positive changes in pressure vs. time (dP/dt), decreasing plasma renin levels, increasing alpha calcitonin gene-related peptide release, and increasing substance P release. In some embodiments, said cell is in vitro. In some embodiments, said activity is selected from the group consisting of increasing alpha calcitonin gene-related peptide release, increasing substance P release, and increasing Ca release. In some embodiments, the method further provides a cell comprising an impaired Transient Receptor Potential Vanilloid-1 (TRPV1) receptor and a step d) determining the activity of said cell in the presence of said test composition, wherein said activity is decreased as compared to the activity of a cell expressing a Transient Receptor Potential Vanilloid-1 (TRPV1) receptor. In some embodiments, said Transient Receptor Potential Vanilloid-1 (TRPV1) receptor impaired cell is a Transient Receptor Potential Vanilloid-1 (TRPV1) receptor negative cell. In some embodiments, said Transient Receptor Potential Vanilloid-1 (TRPV1) receptor impaired cell is the cell expressing a Transient Receptor Potential Vanilloid-1 (TRPV1) receptor exposed to a Transient Receptor Potential Vanilloid-1 (TRPV1) receptor inhibitor. In some embodiments, the method further provides an inhibitor and exposing said cell expressing Transient Receptor Potential Vanilloid-1 (TRPV1) receptor to said inhibitor and determining the activity of said cell, wherein said activity is decreased. In some embodiments, said inhibitor is a Transient Receptor Potential Vanilloid-1 (TRPV1) receptor inhibitor, wherein said inhibitor is selected from the group consisting of capsazepine and chelerythrine. In some embodiments, said inhibitor is an alpha calcitonin gene-related peptide receptor inhibitor, wherein said inhibitor is CGRP₈₋₃₇ In some embodiments, said method further provides a cell expressing a cannabinoid-1 receptor, and a step for exposing the cell to the test compound and determining cannabinoid-1 receptor activity, wherein said exposing reduces activation of said cannabinoid-1 receptor. In some embodiments, said test compound for reducing activation of said cannabinoid-1 receptor is selected from the group consisting of RP67580. In some embodiments, said test compound is selected from the group consisting of N-arachidonoyl dopamine (NADA), N-oleoyl-dopamine (OLDA), anandamide, Methanandamide (MethA), 20-hydroxyeicosatetraenoic acid (20-HETE), 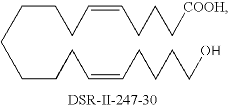
capsaicin (CAP), and derivative or synthetic analog thereof. It is not meant to limit the type of tissue. Indeed, in some embodiments, said tissue is selected from the group consisting of sensory ganglia, cardiac tissue, an aorta, and an artery. In some embodiments, said cell is in a whole organ selected from the group consisting of a heart and a kidney.

### DESCRIPTION OF THE FIGURES

FIG. 1 shows an exemplary three phase responses of mean arterial pressure (MAP) to 5 mg/kg N-arachidonoyl dopamine (NADA) in rats.

FIG. 2 shows an exemplary three phase responses of mean arterial pressure (MAP) of 1 mg/kg compared to 5 mg/kg N-arachidonoyl dopamine (NADA) in rats.

FIG. 3 shows an exemplary mean arterial pressure (MAP) change in rats fed normal salt or high salt diet (NS or HS) in response to vehicle, capsaicin (CAP), and capsazepine (CAPZ)+CAP.

FIG. 4 shows an exemplary mean arterial pressure (MAP) change in response to different doses of N-arachidonoyl dopamine (NADA) in rats fed normal salt or high salt (NS or HS) diets.

FIG. 5 shows an exemplary change in mean arterial pressure (MAP) in rats fed normal salt or high salt (NS or HS) diets in response to N-arachidonoyl dopamine (NADA) and capsazepine (CAPZ)+NADA.

FIG. 6 shows an exemplary arterial pressure (MAP) change in rats fed normal salt or high salt (NS or HS) diets and response to 4 mg/kg N-arachidonoyl dopamine (NADA), capsazepine (CAPZ)+NADA, CGRP₈₋₃₇+NADA, RP67580+NADA.

FIG. 7 shows an exemplary change in arterial pressure (MAP) in response to drug 3 mg/kg N-arachidonoyl dopamine (NADA), capsazepine (CAPZ)+NADA, RP67580+NADA.

FIG. 8 shows exemplary CGRP levels in plasma in response to vehicle and vehicle+N-arachidonoyl dopamine (NADA) (4 mg/kg).

FIG. 9 shows exemplary structures for arachidonic acid, 20-HETE, DSR-II-247-30 (DSR; 20-HEDE), and other derivatives and synthetic analogs.

FIG. 10 shows an exemplary DSR-II-247-30 (DSR) induced-depressor effect is mediated by; (A) a selective TRPV1 antagonist Capsazepine (CAPZ), (B) a selective CGRP receptor antagonist CGRP₈₋₃₇, (C) a neurokinin-1 receptor (NK1) antagonist RP67580. +p<0.05, DSR vs. other groups; *p<0.05, DSR vs. vehicle (n=5-6 rats in each group).

FIG. 11 shows exemplary plasma levels of calcitonin gene-related peptide (CGRP) and substance P induced by intravenous injection of DSR-II-247-30 at dose of 1 mg/kg. +p <0.05 vs. vehicle (n=5-6 rats in each group).

FIG. 12 shows exemplary N-oleoyl-dopamine (OLDA) improved recovery of cardiac function after ischemia and reperfusion (I/R; ISCH) in WT (wild-type; w) but not TRPV1^{-/-} (knock-out; KO; k) hearts by increasing left ventricular developed pressure (LVDP) (WT: 45.2±3.2 vs 61.3±3.8 mmHg; p<0.05; TRPV^{-/-}34.7±2.5 vs 39.8±3.7 mmHg; p>0.05), coronary flow (CF) (WT: 52±4 vs 76±6%, p<0.05; TRPV^{-/}−, 48±4 vs 53±2%, p>0.05), and left ventricular (LV) peak positive dP/dt (+dP/dt) (WT: 2303±214 vs 3227±126 mmHg/s, p<0.05; TRPV^{-/-}1656±119 vs 1967±299 mmHg/s, p>0.05), and by decreasing left ventricular end-diastolic pressure (LVEDP; EPD) (WT: 22.1±1.4 vs 10.1±0.6 mmHg, p<0.05; TRPV^{-/-}28.7±1.7 vs 29.9±3.5 mmHg, #=p>0.05). (with OLDA vs without OLDA, respectively).

FIG. 13 shows exemplary protective effect of N-oleoyl-dopamine (OLDA) in WT (wild-type; w) hearts was abolished by A) Calcitonin-Gene Related Peptide (CGRP₈₋₃₇; K-8-37), B) capsazepine (CAPZ), and C)RP67580 (rp).

FIG. 14 shows an exemplary radioimmunoassay where N-oleoyl-dopamine (OLDA) induced significantly higher A) Calcitonin-Gene Related Peptide (CGRP) and Substance P(SP) release in WT (wild-type; w) compared to TRPV1−/− hearts (p<0.05), B) CAPZ blocked increased CGRP and SP release in WT hearts and C) a Protein Kinase C(PKC) inhibitor, chelerythrine (che) blocked increased CGRP and SP release in WT hearts.

FIG. 15 shows an exemplary Mean arterial pressure and heart rate responses to bolus injection of Narachidonoyl-dopamine (NADA) (1 mg/kg; 4 mg/kg; 10 mg/kg) in rats fed a normal (A) or high (B) salt diet for 10 days. Values are mean ±SE (n=6 to 8). *P<0.05 compared with the corresponding vehicle-treated value.

FIG. 16. shows an exemplary A, Changes in mean arterial pressure (MAP) at 8 minutes after bolus injection of N-arachidonoyl-dopamine (NADA) (1 mg/kg; 4 mg/kg; 10 mg/kg) in rats fed a normal-salt (NS) or high-salt (HS) diet for 10 days. B, MAP responses to bolus injection of NADA (4 mg/kg) with or without capsazepine (CAPZ), CGRP₈₋₃₇ or SR141716A in rats fed a NS or HS diet for 10 days. Values are mean ±SE (n=6 to 8). *P<0.05 compared with the corresponding vehicle-treated rats. †P<0.05 compared with NS treated rats at the same dose of NADA. ‡P<0.05 compared with the corresponding rats treated with NADA alone.

FIG. 17 shows an exemplary A, Mean arterial pressure (MAP) responses to bolus injection of capsaicin (CAP) (10 μg/kg; 30 μg/kg) with or without capsazepine (CAPZ) (3 mg/kg) in urethane anesthetized rats fed a normal-salt (NS) or high-salt (NS) diet for 10 days. B, MAP responses to bolus injection of calcitonin gene-related peptide (CGRP) (0.5 μg/kg; μg/kg; 5 μg/kg) in conscious rats fed a NS or HS diet for 10 days. Values are mean ±SE (n=7 to 8). *P<0.05 compared with the corresponding vehicle-treated rats. †P<0.05 compared with NS-treated rats at the same dose of CAP or CGRP. ‡P<0.05 compared with the corresponding rats treated with CAP at the same dose.

FIG. 18 shows exemplary effects of N-arachidonoyl-dopamine (NADA) (1 μM; 10 μM) on calcitonin gene-related peptide (CGRP) release with or without capsazepine (CAPZ) (10 μM) in mesenteric artery isolated from rats fed a normal-salt (NS) or high-salt (HS) diet for 10 days. Values are mean ±SE (n=7 to 8). *P<0.05 compared with the corresponding vehicle-treated rats. †P<0.05 compared with NS-treated rats at the same dose of NADA. ‡P<0.05 compared with the corresponding rats treated with NADA (10 μM) alone.

FIG. 19 shows an exemplary plasma calcitonin gene-related peptide (CGRP) levels in response to injection of vehicle or N-arachidonoyl-dopamine (NADA) (4 mg/kg) in rats fed a normal-salt (NS) or high-salt (HS) diet for 10 days. Values are mean ±SE (n=6 to 8). *P<0.05 compared with the corresponding vehicle-treated rats. †P<0.05 compared with the corresponding value in NS-treated rats.

FIG. 20 shows an exemplary Western blot analysis showing the TRPV1 protein expression in mesenteric arteries in rats fed a normal-salt (NS) or high-salt (HS) diet for 10 days. Values are mean ±SE (n=4 to 5). *P<0.05 compared with NS-treated rats.

FIG. 21 shows an exemplary Time course responses of mean arterial pressure (A) and heart rate (B) to bolus injection of capsazepine (3 mg/kg) in rats fed a normal (NS) or high (HS) salt diet for 3 weeks. Values are mean ±SE (n=6 to 7)

FIG. 22 shows exemplary Peak changes in mean arterial pressure (A) and heart rate (B) after bolus injection of capsazepine (3 mg/kg) in rats fed a normal (NS) or high (HS) salt diet for 3 weeks. Values are mean ±SE (n=6 to 7). *P<0.05 compared with NS-treated rats.

FIG. 23 shows exemplary Mean arterial pressure (MAP) responses to intravenous injection of calcitonin generated peptide (CGRP, 1 μg; 5 μg/kg) in conscious rats fed a normal (NS) or (HS) salt diet for 3 weeks. Values are mean ±SE (n=5 to 6). *P<0.05 compared with the corresponding vehicle treated values. †P<0.05 compared with NS-treated rats at the same dose of CGRP.

FIG. 24 shows exemplary effects of methanandamide (MethA, 0.1 μM; 10 μM) on calcitonin gene-related peptide (CGRP) release with or without capsazepine (CAPZ) in mesenteric artery isolated from rats fed a normal (NS) or high (HS) salt diet for 3 weeks. Values are mean ±SE (n=7 to 8). *P<0.05 compared with the corresponding vehicle-treated values. †P<0.05 compared with Ns treated rats at the same dose of MethA. ‡P<0.05 compared with the corresponding rats treated with MethA (10 μM).

FIG. 25 shows an exemplary representative ion chromatogram of rats fed a normal (NS) (A) or high (HS)(B) salt diet for 3 weeks. (C) Plasma anandamide (AEA) levels in rats fed a NS or HS diet for 3 weeks. *P<0.05 compared with NS-treated rats.

FIG. 26 shows an exemplary plasma CGRP levels in response to intravenous injection of vehicle or methanandamide (MethA, 5 mg/kg) in rats fed a NS or HS diet for 3 weeks. Values are mean ±SE (n=7 to 8). *P<0.05 compared with the corresponding value in NS-treated rats. †P<0.05 compared with vehicle-treated rats.

FIG. 27 shows an exemplary Western blot analysis showing receptor activity-modifying protein 1 (RAMP1)(A) and calcitonin receptor-like receptor (CRLR)(B) in mesenteric arteries in rats fed a normal (NS) or high (HS) salt diet for 3 weeks. Values are mean ±SE (n=5 to 6). *P<0.05 compared with NS-treated rats.

FIGS. 28A, B, C, D and E show an exemplary effect of three 5-min preconditioning cycles (3PC) on cardiac function at the end of I/R. WT and TRPV1−/− hearts were retrogradely perfused in a Langendorff apparatus, and subjected to 3PC and then I/R (WTpc and TRPV1−/−pc). Hearts were paced at 400 bpm during the initial equilibration period. Pacing was terminated during ischemia and reinitiated at 3 minutes into the reperfusion period. As ischemia controls, WT and TRPV1−/− hearts were equilibrated for 55 minutes, followed by I/R (WTi and TRPV1−/−i). A, Left ventricular end-diastolic pressure (LVEDP). B, Left ventricular developed pressure (LVDP). C, % coronary flow recovery (% CF). D, LV peak positive dP/dt (+dP/dt). E, the peak negative dP/dt (−dP/dt). Values are mean ±SEM; n=7-8; *P<0.05 vs WTpc; †P<0.05 vs TRPV1−/− pc; ‡P<0.05 vs WTi hearts. FIG. 28B shows an exemplary effect of 3PC on cardiac function during I/R. WT- and TRPV1−/− hearts were retrogradely perfused in a Langendorff apparatus and then subjected to 3PC followed by I/R. (WTpc and TRPV1−/−pc). Hearts were paced at 400 bpm during the initial equilibration period. Pacing was terminated during ischemia and reinitiated at 3 minutes into the reperfusion period. As normal controls, WT and TRPV1−/− hearts were perfused and paced throughout the 130-minute period (WTn and TRPV1−/−n); As ischemia controls, WT and TRPV1−/− hearts were equilibrate for 55 min, followed by I/R (WTi and TRPV1−/−i). A, Left ventricular end-diastolic pressure (LVEDP). B, Left ventricular developed pressure (LVDP). C, % coronary flow recovery (% CF). D, LV peak positive dP/dt (+dP/dt). E, the peak negative dP/dt (−dP/dt). Values are mean ±SEM; n=5-8; *P<0.05 vs WTpc.

FIGS. 29A, B, C, D and E show exemplary effects of blockade of the TRPV1 receptor by capsazepine (CAPZ) on PC induced cardiac protection at the end of I/R. WT and TRPV1−/− hearts were subjected to the same PC and I/R protocol described in FIG. 1. CAPZ was added to the perfusate 5 minutes before PC. A, Left ventricular end-diastolic pressure (LVEDP). B, Left ventricular developed pressure (LVDP). C, % coronary flow recovery (% CF). D, LV peak positive d P/dt (+dP/dt). E, the peak negative dP/dt (−dP/dt). Values are mean ±SEM; n=6-8; *P<0.05 vs WTpc. FIG. 29B shows an exemplary effects of blockade of the TRPV1 receptor by capsazepine (CAPZ) on PC induced cardiac protection during I/R. WT and TRPV1−/− hearts were subjected to the same PC and I/R protocol described in FIG. 1. CAPZ was added to the perfusate 5 minutes before PC. A, Left ventricular end-diastolic pressure (LVEDP). B, Left ventricular developed pressure (LVDP). C, % coronary flow recovery (% CF). D, LV peak positive d P/dt (+dP/dt). E, the peak negative dP/dt (−dP/dt). Values are mean ±SEM; n=6-8; *P<0.05 vs WTpc.

FIGS. 30A, B, C, D and E show an exemplary effects of the CGRP receptor antagonist, CGRP8-37, on PC induced cardiac protection at the end of I/R. WT and TRPV1−/− hearts were subjected to the same PC and I/R protocol described in FIG. 1. CGRP8-37 was added to the perfusate 5 minutes before PC. A, Left ventricular end-diastolic pressure (LVEDP). B, Left ventricular developed pressure (LVDP). C, % coronary flow recovery (% CF). D, LV peak positive d P/dt (+dP/dt). E, the peak negative dP/dt (−dP/dt). Values are mean ±SEM; n=6-8; *P<0.05 vs WTpc. FIG. 30B shows exemplary effects of the CGRP receptor antagonist, CGRP₈₋₃₇, on PC induced cardiac protection during I/R. WT and TRPV1−/− hearts were subjected to the same PC and I/R protocol described in FIG. 1. CGRP8-37 was added to the perfusate 5 minutes before PC. A, Left ventricular end-diastolic pressure (LVEDP). B, Left ventricular developed pressure (LVDP). C, % coronary flow recovery (% CF). D, LV peak positive dP/dt (+dP/dt). E, the peak negative dP/dt (−dP/dt). Values are mean ±SEM; n=6-8; *P<0.05 vs WTpc.

FIGS. 31A, B, C, D, E show exemplary effects of the SP receptor antagonist, RP67580 (RP), on PC induced cardiac protection at the end of I/R. WT and TRPV1−/− hearts were subjected to the same PC and I/R protocol described in FIG. 1. RP was added to the perfusate 5 minutes before PC. A, Left ventricular end-diastolic pressure (LVEDP). B, Left ventricular developed pressure (LVDP). C, % coronary flow recovery (% CF). D, LV peak positive d P/dt (+dP/dt). E, the peak negative dP/dt (−dP/dt). Values are mean ±SEM; n=6-8; *P<0.05 vs WTpc. FIG. 31B shows exemplary effects of the SP receptor antagonist, RP67580 (RP), on PC induced cardiac protection during I/R. WT and TRPV1−/− hearts were subjected to the same PC and I/R protocol described in FIG. 1. RP was added to the perfusate 5 minutes before PC. A, Left ventricular end-diastolic pressure (LVEDP). B, Left ventricular developed pressure (LVDP). C, % coronary flow recovery (% CF). D, LV peak positive d P/dt (+dP/dt). E, the peak negative dP/dt (−dP/dt). Values are mean ±SEM; n=6-8; *P<0.05 vs WTpc.

FIG. 32 shows exemplary effects of substance P(SP) from isolated hearts subjected to three 10-min preconditioning cycles (3PC), entailing 10 min of anaerobic oxygen followed by 10 min of aerobic oxygen in WT and TRPV1−/− mice in the absence or presence of the TRPV1 receptor antagonist, capsazepine (CAPZ). Values are mean ±SEM; n=4; *P<0.05 vs WTn; †P<0.05 vs WTpc.

FIG. 33 shows an exemplary release of CGRP from isolated hearts subjected to three 10-min preconditioning cycles (3PC), entailing 10 min of anaerobic oxygen followed by 10 min of aerobic oxygen in WT and TRPV1−/− mice in the absence or presence of the TRPV1 receptor antagonist, capsazepine (CAPZ), Values are mean ±SEM; n=4 *P<0.05 vs WTn; †P<0.05 vs WTpc.

FIG. 34 shows an exemplary cardiac injury as assessed by the release of lactate dehydrogenase (LDH) during I/R. WT and TRPV1−/− hearts were retrogradely perfused in a Langendorff apparatus and subjected to 3PC and followed by I/R. Effects of blockade of TRPV1, CGRP, and SP receptors by capsazepine (CAPZ), CGRP8-37, and RP67580 (RP), respectively, on LDH release in WT and TRPV1−/− hearts were assessed. Coronary outflow was collected during the first period of 10 min to 20 min of I/R and sampled for the LDH content. (A) WT and TRPV1−/− hearts subjected to 3PC; (B) WT and TRPV1−/− hearts subjected to 3PC+CAPZ; (C) WT and TRPV1−/− hearts subjected to 3PC+CGRP8-37; (D) WT and TRPV1−/− hearts subjected to 3PC+RP; Values are mean ±SEM; n=6-8; *P<0.05 vs WTpc; †P<0.05 vs WTi; ‡P<0.05 vs TRPV1−/−pc.

FIG. 35 shows an exemplary natriuretic, diuretic and depressor effects observed 10 min after administration of trichlormethiazide (10 mg/kg, intravenously), at which time the maximal natriuresis and diuresis were seen at the same time as the maximal reductions in blood pressure. (a) Urine flow rate. (b) Urine sodium excretion. N¼5, P<0.001 versus vehicle of corresponding group; †P<0.01 versus trichlormethiazide of control rats with a normal sodium diet (Con-NS); trichlormethiazide of capsaicin-pretreated rats with a normal sodium diet (Cap-NS) and trichlormethiazide of control rats with a high sodium diet (Con-HS). (c) Mean arterial pressure. N¼5,_P<0.01 versus vehicle of Con-NS, Cap-NS and Con-HS rats; †P<0.01 versus trichlormethiazide of Con-NS, Cap-NS and Con-HS rats; #P<0.01 versus vehicle of capsaicin-pretreated rats with a high sodium diet (Cap-HS). Vehicle; trichlormethiazide.

FIG. 36 shows an exemplary natriuretic, diuretic and depressor effects observed 5 min after the administration of furosemide (1 mg/kg, intravenously), at which time the maximal natriuresis and diuresis were seen at the same time as the maximal reductions in blood pressure. (a) Urine flow rate. (b) Urine sodium excretion. N¼6,_P<0.001 versus vehicle of corresponding group; †P<0.01 versus furosemide of control rats with a normal sodium diet (Con-NS), #P<0.01 versus furosemide of capsaicinpretreated rats with a normal sodium diet (Cap-NS), $P<0.05 versus furosemide of control rats with a high sodium diet (Con-HS). (c) Mean arterial pressure. N¼6,_P<0.001 versus vehicle of Con-NS, Cap-NS and Con-HS rats; †P<0.001 versus furosemide of Con-NS, Cap-NS and Con-HS rats; #P<0.001 versus vehicle of capsaicin-pretreated rats with a high sodium diet (Cap-HS). Vehicle; furosemide.

FIG. 37 shows an exemplary natriuretic, diuretic and depressor effects observed 10 min after the administration of amiloride (1 mg/kg, intravenously), at which time the maximal natriuresis and diuresis were seen at the same time as the maximal reductions in blood pressure. (a) Urine flow rate. (b) Urine sodium excretion. N¼5,_P<0.001 versus vehicle of corresponding group. (c) Mean arterial pressure. N¼5,_P<0.001 versus vehicle of control rats with a normal sodium diet (Con-NS), vehicle of capsaicinpretreated rats with a normal sodium diet (Cap-NS) and vehicle of control rats with a high sodium diet (Con-HS); †P<0.01 versus amiloride of Con-NS, Cap-NS and Con-HS rats. Vehicle; amiloride.

FIG. 38 shows an exemplary protein levels of thiazide-sensitive NaCl co-transporter (NCC), bumetamide-sensitive type 2 Na†K†2Cl co-transporter (NKCC) and amiloridesensitive epithelial sodium channel a-subunit (a-ENaC) in the kidneys. The protein abundance was normalized with b-actin. (a) NCC abundance in the renal cortex. N¼5,_P<0.05 versus control rats with a normal sodium diet (Con-NS), †P<0.01 versus capsaicin-pretreated rats with a normal sodium diet (Cap-NS) and control rats with a high sodium diet (Con-HS). (b) NKCC2 abundance in the renal medulla. N¼5,_P<0.01 versus Con-NS rats, †P<0.01 versus Cap-NS rats, #P<0.05 versus Con-HS rats. (c) NKCC2 abundance in the renal cortex. N¼5,_P<0.01 versus Con-NS rats, †P<0.05 versus Cap-NS rats, #P<0.01 versus Con-HS rats. (d) a-ENaC abundance in the renal cortex. N¼5, P>0.05.

FIG. 39 shows exemplary effects of capsaicin with or without capsazepine on calcitonin generelated peptide (CGRP) release from renal tissues. N¼5,_P<0.05 versus vehicle, capsazepine plus capsaicin and capsazepine; †P<0.001 versus capsaicin of control rats with a normal sodium diet (Con-NS) and capsaicin of control rats with a high sodium diet (Con-HS). Vehicle; capsaicin; capsazepine and capsaicin; capsazepine.

FIG. 40 shows an exemplary systolic blood pressure in vehicle (CON) or capsaicin (CAP)-treated rats fed a normal (NS) or high (HS)-sodium diet. Values are means SE; n=6-7 rats. *P<0.05 vs. CON-HS, CON-NS, and CAP−NS.

FIG. 41 shows an exemplary renal cortical (A) and medullary (B) superoxide levels in CON or CAP-treated rats fed a NS or HS diet with or without apocynin (APO). Values are means SE; n=7-8 rats. *P<0.05 vs. CON-HS, CON-NS, and CAPNS. #P<0.05 vs. CAP−HS.

FIG. 42 shows exemplary plasma levels (A) and 24-h urine excretion (B) of 8-iso-prostaglandin F2_(8-isoprostane) in CON or CAP-treated rats fed a NS or HS diet. Values are means SE; n=6-7 rats. *P<0.05 vs. CON-NS and CAPNS. #P<0.05 vs. CON-HS.

FIG. 43 shows an exemplary renal Cu/Zn SOD (A: cortex; B: medulla) and Mn SOD (C: cortex; D: medulla) activity in CON or CAP-treated rats fed a NS or HS diet. Values are means SE; n=7-8 rats. *P<0.05 vs. CON-NS and CAP−NS.

FIG. 44 shows an exemplary renal Cu/Zn SOD (A: cortex; B: medulla) and Mn SOD (C: cortex; D: medulla) protein expression in CON or CAP-treated rats fed a NS or HS diet. Values are means SE; n=4-5 rats. *P<0.05 vs. CON-NS and CAP−NS.

FIG. 45 shows an exemplary renal p47phox (A: cortex; B: medulla) and gp91phox (C: cortex; D: medulla) protein expression in CON or CAP-treated rats fed a NS or HS diet. Values are means SE; n=4-5 rats. *P<0.05 vs. CON-HS, CON-NS and CAP−NS.

FIG. 46 shows an exemplary creatinine clearance (A) in CON or CAP-treated rats fed a NS or HS diet, and relationship between creatinine clearance and renal cortical superoxide level (B) in CAP treated rats fed a HS diet. *P<0.05 vs. CON-HS, CON-NS, and CAP−NS.

FIG. 47 shows an exemplary time course of systolic blood pressure measured by the tail-cuff method. Systolic blood pressure in DR or DS rats fed an LS or HS diet for 3 weeks. Values are mean +/−SE (n=7 to 8). *P<0.05 compared with corresponding LS diet group.

FIG. 48 shows an exemplary time course responses of MAP (A) and HR (B) to bolus injection of CAPZ (3 mg/kg) in DR or DS rats fed an LS or HS diet for 3 weeks. Values are mean +/−SE (n=6 to 7). *P<0.05 compared with corresponding LS diet group.

FIG. 49 shows an exemplary MAP responses to intravenous injection of capsaicin (CAP, 10 μg/kg and 30 μg/kg) with or without CAPZ (3 mg/kg) in urthane-anesthetized DR or DS rats fed an LS or HS diet for 3 weeks. Values are mean +/−SE (n=5 to 6). *P<0.05 compared with corresponding LS-treated rats at the same dose of CAP. †P<0.05 compared with corresponding DR or DS rats treated with CAP at the dose of 10 μg/kg. #P P<0.05 compared with corresponding DR or DS rats treated with CAP at the dose of 30 μg/kg.

FIG. 50 shows an exemplary immunoactive CGRP content in DRG of DR or DS rats fed an LS or HS diet for 3 weeks. Values are mean +/−SE (n=6 to 7). *P<0.05 compared with corresponding LS diet group.

FIG. 51 shows an exemplary western blot analysis showing the TRPV1 protein expression in mesenteric arteries in DR or DS rats fed an LS or HS diet for 3 weeks. Values are mean +/−SE (n=4 to 5). *P<0.05 compared with corresponding LS diet group.

FIG. 52 shows an exemplary western blot analysis showing the TRPV1 protein expression in renal cortex (A) and medulla (B) in DR or DS rats fed an LS or HS diet for 3 weeks. Values are mean +/−SE (n=4 to 5). *P<0.05 compared with corresponding LS diet group.

FIG. 53 shows an exemplary dose-related effects of unilateral intramedullary infusion of methanandamide (MethA) on mean arterial pressure (MAP) and renal blood flow in anesthetized Wistar rats (n¼5). *P<0.05 versus vehicle (veh) or MethA at 15 nmol/kg per min group, +P<0.05 versus MethA at 150 nmol/kg per min group.

FIG. 54 shows an exemplary dose-related effects of unilateral intramedullary infusion of methanandamide (MethA) on urine flow rate and urine sodium excretion in anesthetized Wistar rats (n¼5). *P<0.05 versus vehicle (veh) or MethA at 15 nmol/kg per min group. Open bars, infused kidney; shaded bars, contralateral kidney.

FIG. 55 shows an exemplary effects of unilateral intramedullary infusion of methanandamide (MethA, 300 nmol/kg per min) on urine flow rate with or without ipsilateral denervation in conjunction with infusion of capsazepine (Capz, 150 nmol/kg per min) or AM251 (Am, 150 nmol/kg per min) in anesthetized Wistar rats (n ¼6-8). *P <0.05 versus vehicle (veh); +P<0.05 versus Capz-MethA; #P<0.05 versus Am-MethA.

FIG. 56 shows exemplary effects of unilateral intramedullary infusion of methanandamide (MethA, 300 nmol/kg per min) on mean arterial pressure (MAP) with or without ipsilateral denervation, in conjunction with infusion of capsazepine (Capz, 150 nmol/kg per min) or AM251 (Am, 150 nm ol/kg per min) in anesthetized Wistar rats (n¼6-8). *P <0.01 versus vehicle (veh), +P<0.01 versus Capz-MethA, #P<0.01 versus denervation-MethA.

FIG. 57 shows exemplary effect of hypertonic saline given via left renal pelvis perfusion (LRPP) on diuresis and natriuresis. NaCl was given at doses of 150, 300, 600 mM via LRPP, and urine flow rate (V, FIG. 57a) and urinary sodium excretion (UNaV, FIG. 157b) of the contralateral kidney are shown (n=5-6 in each group). +vs baseline and different doses of NaCl; +p<0.05.

FIG. 58 shows an exemplary effect of KCl given via left renal pelvis perfusion (LRPP) on diuresis and natriuresis. KCl was given at doses of 150, 300, 600 mM via LRPP, and urine flow rate (V, FIG. 2a) and urinary sodium excretion (UNaV, FIG. 2b) of the contralateral kidney are shown (n=5-6 in each group). +vs baseline and different doses of NaCl; +p<0.05.

FIG. 59 shows exemplary effects of capsazepine (CAPZ), renal denervation (RD) and RP67580 on hypertonic saline or KCl-induced diuresis and natriuresis when given via left renal pelvis perfusion (LRPP). Urine flow rate (V, FIG. 3a) and urinary sodium excretion (UNaV, FIG. 3b) of the contralateral kidney are shown (n=5-6 in each group). +vs baseline and all other groups, +p<0.05.

FIG. 60 shows exemplary effects of hypertonic saline, capsazepine (CAPZ) and RP67580 perfused into left renal pelvis (LRPP) on the ipsilateral afferent renal nerve activity (ARNA). Ipsilateral ARNA is shown (n=5-6 in each group). ++ vs basal and all other groups, ++ p<0.01

FIG. 61 shows exemplary levels of substance P released from isolated renal pelvis in vitro. +p<0.05 vs other unmarked groups (n=5-6 in each group)

FIG. 62 shows an exemplary role of the transient receptor potential vanilloid type 1 (TRPV1) channels in electrical field stimulation (EFS)-induced relaxation in mouse mesenteric resistance arteries. (a) Representative EFS (100 V, 0.5 ms, 20 Hz for 5 s)-induced dilation after incubation with guanethedine (5 mmol/l) and atropine (1 mmol/l) to block the sympathetic and parasympathetic nerves, respectively, and with phenylephrine (PE, 10 mmol/l) to preconstrict the artery of a wild-type (WT) mouse. (b) Representative EFS-induced dilation in the artery of a TRPV1 gene knockout (TRPV^{-/-}) mouse. (c) Representative EFS induced dilation after pretreatment with capsazepine (CAPZ, 10-6 mol/l) in the artery of a WT mouse. (d) Representative EFS-induced dilation after pretreatment with CAPZ in the artery of a TRPV1−/− mouse. (e) Statistical results in each of the experimental groups. Values are mean +/−SEM; *P<0.05 versus the WT group.

FIG. 63 shows an exemplary role of endogenous calcitonin gene-related peptide (CGRP) in electrical field stimulation (EFS)-induced relaxation in mouse mesenteric resistance arteries. (a) Representative EFS-induced dilation after pretreatment with a CGRP receptor antagonist, CGRP8-37 (10-6 mol/l), in the artery of a wild-type (WT) mouse. (b) Representative EFS-induced dilation after pretreatment with CGRP8-37 in the artery of a TRPV1 gene knockout (TRPV1−/−) mouse. (c) Statistical results in each of the experimental groups (the data for WT and TRPV1−/− mice are the same as FIG. 1). Values are mean +/−SEM; +P<0.05 versus WT group.

FIG. 64 shows an exemplary role of endogenous substance P(SP) in electrical field stimulation (EFS)-induced relaxation in mouse mesenteric resistance arteries. (a) Representative EFS-induced dilation after pretreatment with a neurokinin 1 (NK1) receptor antagonist, RP67580 (10-6 mol/l), in the artery of a wild-type (WT) mouse. (b) Representative EFS-induced dilation after pretreatment with RP67580 in the artery of a TRPV1 gene knockout (TRPV1−/−) mouse. (c) Statistical results in each of the experimental groups (the data for WT and TRPV1−/− mice are the same as FIG. 1). Values are mean +/−SEM; +P<0.05 versus WT group.

FIG. 65 shows an exemplary release of calcitonin gene-related peptide (CGRP) from isolated mesenteric resistance arteries of wild-type (WT) and TRPV1 gene knockout (TRPV1−/−) mice under the control condition (WTn and TRPV1−/−n) or subjected to electrical field stimulation (EFS) in the absence or presence of a TRPV1 antagonist, capsazepine (CAPZ). Values are mean +/−SEM; n¼4 for each group; *P<0.05, **P<0.01 versus control WT arteries (WTn); †P<0.05 versus control TRPV1−/− arteries (TRPV1−/− n); ‡P<0.05 versus WT arteries subjected to EFS in the absence of CAPZ (WT#EFS).

FIG. 66 shows exemplary effects of exogenous calcitonin gene-related peptide (CGRP) (10-7, 10-8 and 10-9 mol/l) on vascular relaxation of mesenteric resistance arteries in wild-type (WT) and TRPV1 gene knockout TRPV1−/− mice. Values are mean +/−SEM; n¼4 for each group; *P<0.05 versus WT arteries treated with CGRP 10-9 mol/l; †P<0.05 versus TRPV1−/− arteries treated with CGRP 10-9 mol/l.

FIG. 67 shows an exemplary confocal microscopic images of double immunofluorescence staining of mesenteric arteries isolated from wild type (a-c) or TRPV1−/− mice (d-f). a and d, FITC-labelled TRPV shown in wild type (a) but not TRPV1−/− (d) vessels; b and e, Cy3-labelled CGRP staining shown in both wild type (b) and TRPV1−/− (e) vessels; c and f, double staining of TRPV1−/− and CGRP shown in wild type (c) but not TRPV1−/− (f) vessels. Scale bars, 100 mm.

### DEFINITIONS

To facilitate an understanding of the present invention, a number of terms and phrases as used herein are defined below:

The use of the article “a” or “an” is intended to include one or more.

As used herein, terms defined in the singular are intended to include those terms defined in the plural and vice versa.

As used herein, “transient receptor potential,” and “TRP,” refers to terms appended to at least three classes of ion channels that mediate the response of a cell to external stimuli (electrical charge, agents, and forces) by increasing or decreasing its selective permeability to particular ions, for example, “TRPC” or “canonical,” “TRPV1-6” or “vanilloid,” and “TRPM” or “melatasin.” For purposes of the present invention vanilloid receptor-related TRP channels “TRPV1-6” represent sequence specific and/or assembly specific ion channels that mediate thermosensation and/or pain perception and/or ion entry and/or epithelial Ca²⁺ entry.

As used herein, “transient receptor potential vanilloid Type 1” or “TRPV-1” or “VR1” or “vanilloid-1 receptor” refers to an ion channel comprising sequences such as demonstrated by GenBank nonlimiting examples: Accession AF029310 for Rattus norvegicus (Norway rats) and NM_{—}018727 for Homo sapiens (human). For the purposes of the present invention, TRPV1 is a nonselective cation channel wherein activating a TRPV-1, such as with capsaicin, causes the release of sensory neurotransmitters including but not limited to substance P(SP) and calcitonin gene-related peptide (CGRP) that may lead to altered cardiovascular responses. Activation of TRPV-1 ion channels are associated with regulating mean arterial pressure (MAP), extracellular ion concentration and pain.

As used herein “therapeutic agent,” “agent,” “compound,” or “drug” is used herein to denote a compound or mixture of chemical compounds, a biological macromolecule, or an extract made from biological materials such as bacteria, plants, fungi, or animal (particularly mammalian) cells or tissues that are suspected of having therapeutic properties. Encompassed within this definition are compound analogs, naturally occurring, synthetic and recombinant pharmaceuticals, hormones, neurotransmitters, etc. The compound, agent or drug may be purified, substantially purified or partially purified.

As used herein “carrier” or “excipient” in reference to a pharmaceutical refers to a substance used for the administration of a pharmaceutically active substance may be, for example, either a solid or liquid. In one embodiment, a pharmaceutical carrier may decrease a side-effect of a certain therapeutic agent.

As used herein “side-effect reducing agent” refers to a compound for reducing a side effect of a therapeutic compound, in particular, for reducing an unwanted side effect such as crying or laughing, for example, a CB1 antagonist compound for reducing the side effects of a therapeutic of the present inventions.

As used herein “wetting agent” refers to a substance that reduces the surface tension of a liquid, for example, causing the liquid to spread across or penetrate more easily the surface.

As used herein “targeting agent” in reference “for sensory nerves” refers to an agent for preferential delivery of a therapeutic to a tissue or cell, in particular to sensory nerves or TRPV1 cells, for example, a neurotropic compound or TRPV1+ cell binding compound.

The term “synthetic”, as used herein, refers to any non-biological chemical reaction process. When used in reference to a “synthetic compound” synthetic refers to a non-biologically produced compound.

As used herein “analog” refers to a synthetic compound that exactly matches a naturally occurring compound, or provides a similar biological function as a naturally occurring compound, or provides increased activity as compared to its naturally occurring compound, or provides a desired activity as compared to its naturally occurring compound. Nonlimiting examples of analogs include an analog of arachidonic acid or an analog of 20-HETE, such as 20-HEDE.

As used herein “arachidonic acid,” “AA,” or “20:4(n-6)” refers to an omega-6 fatty acid with the structure “5,8,11,14-Eicosatetraenoic acid” or “(all-Z)-5,8,11,14-Eicosatetraenoic acid” with a chemical formula of C₂₀—H₃₂—O₂ and a structure of:

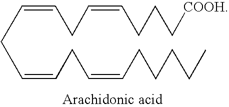

As used herein, the phrase “derivatives of arachidonic acid” refers to chemical compositions comprising arachidonic acid with a chemical group attached, including (but Not limited to) amide groups, for example 20-HETE, which has the following structure:

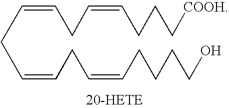

As used herein “agonist” refers to molecules or compounds which mimic the action of a “native” or “natural” compound, for example, anandamide. Agonists may or may not be homologous to these natural compounds in respect to conformation, charge or other characteristics. Thus, agonists may or may not be recognized by, e.g., receptors expressed on cell surfaces. In any event, regardless if the agonist is recognized by a natural compound in a manner similar to a “natural” compound or molecule, the agonist may cause physiologic and/or biochemical changes within the cell, such that the cell reacts to the presence of the agonist in the same manner as if the natural compound was present. Nonlimiting examples of agonists for TRPV1 ion channels (TRPV1) of the present invention are DSR-II-247-30 (DSR; 20-HEDE), N-arachidonoyl dopamine (NADA), anandamide, 20-hydroxyeicosatetraenoic acid (20-HETE) and capsaicin (CAP).

As used herein, “anandamide,” “arachidonoylethanolamide,” or “AEA” refer to an endocannabinoid neurotransmitter “(5Z, 8Z, 11Z, 14Z)-N-(2-hydroxyethyl) icosa-5,8,11,14-tetraenamide” of the chemical formula” C₂₂H₃₇NO₂.”

As used herein, “N-arachidonoyl dopamine” “NADA” refers to a capsaicin-like endogenous ligand of vanilloid (VR1) receptors.

As used herein, “capsaicin” and “CAP” refers to a 8-methyl-N-vanillyl-6-nonenamide)” and “(E)-N-(4-hydroxy-3-methoxybenzyl)-8-methylnon-6-enamide” of the chemical formula “C₁₈H₂₇NO₃.”

As used herein, “DSR-II-247-30,” “DSR,” and “20-HEDE” refers to a synthetic analog of 20-hydroxyeicosatetraenoic acid (20-HETE) with the following structure:

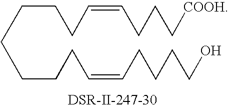

As used herein, “20-HETE agonists” and “20-HETE antagonists” refer to molecules comprising a carboxyl or an ionizable group on carbon 1 and a double bond near the 14 or 15 carbon wherein 20-HETE agonists further comprise a functional group capable of hydrogen bonding on carbon 20 or 21, whereas 20-HETE antagonists lack this reactive group.

As used herein “antagonist” refers to molecules or compounds which inhibit the action of a “native” or “natural” compound. Antagonists may or may not be homologous to these natural compounds in respect to conformation, charge or other characteristics. Thus, antagonists may be recognized by the same or different receptors or molecules that are recognized by an agonist. Antagonists may have allosteric effects which prevent the action of an agonist (e.g., by modifying a DNA adduct, or antagonists may prevent the function of the agonist (e.g., by blocking a DNA repair molecule).

As used herein, “capsazepine” and “CAPZ” refers to a synthetic analog of capsaicin that acts as a specific capsaicin antagonist (A.G. Scientific, Inc.) of chemical formula “C₁₉H₂₁C₁N₂O₂S” and “N-[2-(4-Chlorophenyl)ethyl]-1,3,4,5-tetrahydro-7,8-dihydroxy-2H-2-benzazepine-2-carbothioamide” (Calbiochem).

The term “receptors” refers to structures expressed by cells and which recognize binding molecules (e.g., ligands).

As used herein, “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject or a rat.

As used herein, “activating a transient receptor potential vanilloid ion channel” refers to increasing ion transport and/or triggering the release of a secondary molecule, such as CGRP and/or Substance P. For example, increasing ion transport may show a decrease or an increase in MAP or a reduction in symptoms, such as a reduction in symptoms indicative of salt sensitive hypertension, a reduction in analgesia effects, a reduction in pain, a reduction of cardiac symptoms, or a reduction in cardiac tissue damage.

As used herein, symptoms, for example of hypertension and pain, are “reduced” when the magnitude (e.g. intensity) or frequency of symptoms is reduced.

As used herein, analgesia, is the reduction of pain without a loss of consciousness. It is not intended that the present invention is limited to the treatment of any specific type of pain. For example the treatment of sensory nerve pain, orthopedic, muscular, abdominal, urological, and gynecological pain is expressly contemplated. In addition, the treatment of headache (especially migraine headache) is also contemplated. The treatment of “breakthrough pain” is also contemplated. The present invention specifically contemplates treatment such that one or more symptoms are reduced (and the condition of the subject is thereby “improved”), albeit not completely eliminated. The present invention is also not limited to the reduction of all symptoms or all pain.

As used herein, “salt-sensitive subjects” refer to subjects whose blood pressure reacts significantly to salt intake as in “salt sensitive hypertension.” High-salt diets in a subject with “salt-sensitivity” or “salt sensitive hypertension” may harm the heart, kidney, and brain and increase the risk for death, regardless of their blood pressure measurement. Nonlimiting examples of salt-sensitive subjects include genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, a subject with dysfunctional and/or compromised TRPV1 receptors, and a subject with high salt intake.

As used herein, “symptom(s) of salt sensitive hypertension” refer to an increase (over normal values) in blood pressure, mean arteriole pressure, and/or decreased plasma renin levels that correlate with an increased intake of dietary sodium chloride.

As used herein, “a patient is at risk for hypertension” refers to a patient comprising a history of high blood pressure in the family, a family member who suffered from a stroke, a frequent dieter, older than age 45, consumes a high salt diet, children with renal dysfunction, renal deficiency, renal dysfunction, consume more than two alcoholic beverages per day, overweight, Hispanic and African American.

As used herein, “calcitonin gene-related peptide” and “CGRP” refers to a 37-amino acid peptide existing in a(1) and b(2) forms. As used herein, CGRP antagonist includes but is not limited to a BIBN4096BS peptide (Boehringer-Ingelheim (Mannheim, Germany) (Doods et al., 2000, Br J Pharmacol 129: 420-423) and CGRP₈₋₃₇ peptide ((Peninsula Laboratories Inc., Belmont, Calif.).

As used herein, “Substance P” refers to an eleven-amino acid neuropeptide consisting of SEQ ID NO:01 (Arg Pro Lys Pro Gln Gln Phe Phe Gly Leu Met-NH2). An endogenous receptor for Substance P is neurokinin 1 receptor (NK1-receptor, NK1R) while Substance P antagonist (SPA) includes but are not limited to aprepitant and RP67580.

As used herein, “pain” refers to an unpleasant sensation which may be associated with actual or potential tissue damage and which may have physical Nociception and emotional components. Nociception, on the other hand, is a neurophysiological term and denotes specific activity in nerve pathways.

As used herein, “inhibiting a transient receptor potential vanilloid ion channel” refers to reducing ion transport and/or inhibiting the release of a secondary molecule, such as CGRP and/or Substance P.

As used herein “standard injection” refers to the placement of a pharmaceutical composition into a subject (e.g., with a hypodermic needle). For example, such injection can be made subcutaneously, intravenously, intramuscularly, intra-arterial, etc.

As used herein “single dosage” refers to a pharmaceutical composition of a formulation that is capable of achieving its intended effect in a single administration or application.

As used herein “coadministration” refers to administering at least 2 agents to a subject.

As used herein, “symptoms of cardiovascular disease” refers to any clinical manifestation of a disease state associated with the heart and vasculature. For example, said clinical manifestation include: angina pectoris, myocardial infarction, congestive heart failure, cardiomyopathy, hypertension, arterial stenosis, and venous stenosis. The present invention specifically contemplates treatment such that symptoms are reduced (and the condition of the subject is thereby “improved”), albeit not completely eliminated.

As used herein, “Hypertension” refers to an abnormal increase of blood pressure in the arteries continuing over a period of time. It occurs when the arterioles, the small blood vessels that branch off from the arteries, become constricted. This constriction of the arterioles makes it difficult for blood to flow which increases pressure against the artery walls.

A blood pressure reading of approximately 110/60 to 140/90 for humans is considered to be in the normal range. The first number (110) is the systolic pressure that measures the blood pressure in the arteries when the heart is contracting and pumping blood. The second number (60) is the diastolic pressure that measures the blood pressure in the arteries when the heart is at rest. Hypertension adds to the workload of the heart and arteries. Over time, this can lead to heart and blood vessel damage that causes hardening of the arteries, heart failure, stroke, kidneys problems, blindness, and brain damage. In one embodiment of the present invention, a symptom of cardiovascular disease comprises a measured blood pressure of approximately 140/90 or higher. In a preferred embodiment, the diagnosis of said hypertensive blood pressure (e.g. approximately 140/90 or higher) is confirmed by a plurality of measurements of approximately 140/90 or higher spaced over the period of at least two weeks. It is not intended that the present invention be limited by the means by which blood pressure is measured. Moreover, additional symptoms of hypertension also include, but are not limited to, tiredness, confusion, nausea, vomiting, anxiety, excessive perspiration, muscle tremor, chest pain, nosebleed, and buzzing in the ears.

As used herein, “salt sensitive hypertension” refers to hypertension influenced by the level of sodium ions in the blood.

As used herein, “symptoms of cardiovascular disease” refers to any clinical manifestation of a disease state associated with the heart and vasculature. For example, said clinical manifestation include: angina pectoris, myocardial infarction, congestive heart failure, cardiomyopathy, hypertension, arterial stenosis, and venous stenosis. The present invention specifically contemplates treatment such that symptoms are reduced (and the condition of the subject is thereby “improved”), albeit not completely eliminated.

As used herein, the term “patient” refers to a human or non-human organism that is either symptomatic or asymptomatic for cardiovascular disease. Preferably, a human patient is under the supervision of a physician or hospitalized.

### DESCRIPTION OF THE INVENTION

The present inventions relate to therapeutic compositions comprising, and methods utilizing, arachidonic acid derivatives and analogs for treatment of patients demonstrating symptoms of pathological conditions. Specifically, the inventions relate to therapeutic compositions for activating transient receptor potential vanilloid-1 channels (TRPV1). Additionally, therapeutic compositions are provided for increasing TRPV1-responses. These pathological conditions include, but are limited to, hypertension, in particular salt induced hypertension, and cardiovascular complications, including, myocardial infarction, kidney dysfunction, diabetes, and inflammation. Further, the inventions relate to drug screening methods for providing additional therapeutic compounds.

In one embodiment, transient receptor channel type 1 agonists are provided for treating patients at risk for or actually experiencing high blood pressure and/or cardiac and cardiovascular related symptoms. In other embodiments, transient receptor channel type 1 agonists are provided for treating at risk for or actually experiencing renal dysfunction. In yet other embodiments, transient receptor channel type 1 agonists are provided for treating at risk for or actually experiencing end organ damage.

I. Introduction and Overview of the TRP Family and TRPV1.

Members of the TRPV subfamily are mainly implicated in thermosensation (TRPV1-4), (Smith, et al., Nature 418, 186-190 (2002); herein incorporated by reference) but have also been implicated in osmosensation (TRPV4) (Liedtke, et al., Pflugers Arch-Eur J Physiol 451, 176-180 (2005); herein incorporated by reference) and calcium reabsorption in the kidney and the GI tract (TRPV5, 6) (Nijenhuis, et al., Pflugers Arch-Eur J Physiol 451, 181-192 (2005); herein incorporated by reference). Channels in the TRPM subfamily have been identified with Mg2+ absorption (TRPM6) and with sensing cool temperatures (TRPM8) (Reid, Pflugers Arch-Eur J Physiol 451, 250-263 (2005); herein incorporated by reference).

TRPV1, a recently cloned member of the TRPV subfamily, has well recognized roles in pain (Caterina, et al., Nature 389, 816-824 (1997); herein incorporated by reference) and thermosensation (Jordt, et al., Curr Opin Neurobio 13, 487-492 (2003); herein incorporated by reference). TRPV1 participates in particulate matter-induced apoptosis (Agopyan, et al., Am J Physiol Lung Cell Mol Physiol 283, L563-L572 (2004); herein incorporated by reference), normal bladder function, (Birder, et al., Nature Neuroscience 5(9), 856-860 (2002); herein incorporated by reference taste), (Lyall, et al., J Physiol 558(1), 147-159 (2004); herein incorporated by reference) and neurogenic inflammation (Planells-Cases, et al., Pflugers Arch-Eur J Physiol 451, 151-159 (2005); herein incorporated by reference). Intense study has drawn attention to TRPV1's role in the regulation of salt and water homeostasis and its subsequent effect on systemic blood pressure. TRPV1 has a role in the cardiovascular system, particularly in the regulation of salt sensitivity of arterial pressure.

Members of the TRPV family are known to be activated by a wide array of stimuli, including acidity, heat, lipids, phosphorylation, phorbol esters, changes in osmolarity, and others. TRPV channels are the members of the TRP superfamily known to be activated by vanilloids, the property for which the subfamily was named. Because the vanilloid capsaicin is a known activator of TRPV1, the protein is also known as the “capsaicin receptor.” TRPV1 can be activated by multiple stimuli (O'Neil, et al., News Physiol Sci 18, 226-231 (2003); Gunthorpe, et al., TRENDS in Pharmacological Sciences 23(4), 183-191 (2002); herein incorporated by reference) and as a result it was hypothesized that it may function as a “molecular integrator” of biological systems. This hypothesis stems from the observation that protons can lower the threshold for heat activation of TRPV1 (Tominaga, et al., Neuron 21, 531-543 (1998); herein incorporated by reference).

As a result of its complex modes of activation, much study was done on the molecular structure of TRPV1 and its biochemical characterization (Tominaga, et al., Neuron 21, 531-543 (1998); herein incorporated by reference).

When the gene encoding TRPV1 was initially cloned in 1997, it was discovered that TRPV1 cDNA contains a 2,514 nucleotide open reading frame encoding an 838 amino acid peptide with a molecular mass of around 95 kDa (Caterina, et al., Nature 389, 816-824 (1997); herein incorporated by reference). Both termini point intracellularly. TRPV1 was described as having an N-terminus of 432 amino acids that notably contains three ankyrin repeat domains following a proline-rich region. These ankyrin repeats are believed to function as an interconnection of the TRPV1 protein with spectrin-based cytoskeletal elements (Schumacher, et al., J Biol Chem 275(4), 2756-2762 (2000); herein incorporated by reference). Furthermore, it was discovered that calmodulin (CaM) binds to the first ankyrin repeat (residues 189-222) (Tominaga, et al., Pflugers Arch-Eur J Physiol 451, 143-150 (2005); herein incorporated by reference).

The C-terminus has 154 amino acids, although with no currently recognizable motifs. The C-terminus was determined, however, to contain domains responsible for allosteric conformational changes that occur after ligand binding (Vlachova, et al., J Neurosci 23(4), 1340-1350 (2003); herein incorporated by reference). Further study indicated that the C-terminus also contains a 35-amino acid segment (residues 767-801) that is bound by CaM (Numazaki, et al., Proc Natl Acad Sci USA 100(13), 8002-8006 (2003); herein incorporated by reference).

The native quaternary structure of the TRPV1 receptor is composed of at least four equivalent 95 kDa subunits (Numazaki, et al., Proc Natl Acad Sci USA 100(13), 8002-8006 (2003); herein incorporated by reference). This evidence was supported by Kuzhikandathil, et al. when they developed a TRPV1 subunit with a dominant negative mutation through mutation of residues in the sixth transmembrane domain (Kuzhikandathil, et al., J Neurosci 21(22), 8697-8706 (2001); herein incorporated by reference). These mutations resulted in a receptor that was impaired, unable to be activated by capsaicin. Molecular determinants of the association domain of TRPV1 were described in 2004 by Garcia-Sanz, et al. (Garcia-Sanz, et al., J Neurosci 24(23), 5307-5314 (2004); herein incorporated by reference) wherein a stretch of amino acids on the C-terminus spanning Glu684 to Arg721 composes a TRP-like domain similar to the 25-amino acid TRP domains highly conserved in many other TRP channels subunits (Montell, et al., Cell 108, 595-598 (2002); herein incorporated by reference). Deletion of the TRP-like domain from the C-terminus of one TRPV1 subunit significantly inhibited TRPV1 receptor activation by vanilloids, indicating that the TRP-like domain is an association domain that is necessary for the formation of a fully-functional receptor. Given that a deletion mutation can negatively regulate TRPV1 receptor formation, it is not surprising that genomic studies have provided evidence for several splice variant TRPV1 cDNAs that could modulate TRPV1 function (Xue, et al., Genomics 76(1-3), 14-20 (2001); herein incorporated by reference).

When a homomultimer of functional TRPV1 subunits forms, the result is a TRPV1 receptor that functions as a nonselective cation channel that exhibits a time- and Ca2+-dependent outward rectification followed by a long-lasting refractory period (Caterina, et al., Annu. Rev. Neurosci. 24, 487-517 (2001); herein incorporated by reference). In terms of cation selectivity, TRPV1 was shown to have no preference for monovalent cations, but out of divalent cations it prefers calcium over magnesium (Caterina, et al., Nature 389, 816-824 (1997); herein incorporated by reference). Thus when TRPV1 is activated the pore of the channel opens resulting in an influx of extracellular calcium that effects a cellular response.

II. Expression of TRPV1.

Functions of TRPV1 are dependent on its localization within the body and expression within the cell. In Caterina, et al., TRPV1 was reported to be exclusively localized to primary sensory neurons in the dorsal root ganglia (DRG) (Caterina, et al., Nature 389, 816-824 (1997); herein incorporated by reference). In this study evidence indicated that TRPV1 is expressed mainly in small neurons with unmyelinated C fibers. In a 2002 study TRPV1 was also expressed in large neurons with myelinated A6 fibers (Caterina, et al., Annu. Rev. Neurosci. 24, 487-517 (2001); herein incorporated by reference). A more recent study, however, has suggested that TRPV1 is found mainly in the unmyelinated C fibers (Ma, Neurosci. Lett. 319, 87-90 (2002); herein incorporated by reference). Furthermore, these DRG neurons have been classified based on coexpression of TRPV1 with receptors for neurotrophic factors (Kobayashi, et al., J Comp Neurol 493, 596-606 (2005); herein incorporated by reference). The majority of neurons that express trkA, IB4, (Guo, et al., Eur J Neurosci 11, 946-958 (1999); herein incorporated by reference) SP, and CGRP are TRPV1-positive neurons, although the exact phenotype of these neurons varies across species (Funakoshi, et al., Cell Tissue Res 323, 27-41 (2006); herein incorporated by reference). These primary afferent neurons innervate a wide variety of tissues, including the majority of vascular beds. In addition to the dorsal root ganglia, TRPV1 was found in the trigeminal and nodose ganglia.

TRPV1 was found to be present in many normeuronal tissues in both rats and humans. =Study of organs expressing TRPV1 has led to the discovery of novel functions of the TRPV1 receptor that has implications for health and disease (Szallasi, Am J Clin Pathol 118, 110-121 (2002); herein incorporated by reference). Organs of use in the present invention that express TRPV1 include DRG neurons and the CNS39, further including rat organs where TRPV1 protein and/or mRNA were identified are the kidney (Sanchez, et al., Neurosci 107(3), 373-381 (2001); herein incorporated by reference), bladder (Sanchez, et al., Neurosci 107(3), 373-381 (2001); herein incorporated by reference), urothelium (Birder, et al., Proc Nat/Acad Sci USA 98(23), 13396-13401 (2001); Avelino, et al., Neuroscience 109(4) 787-798 (2002); herein incorporated by reference), Heart (McIntyre, et al., Br. J. Pharmacol. 132, 1084-1094 (2001); Zvara, et al., FASEB J 20, 160-162 (2006); herein incorporated by reference, stomach (Sanchez, et al., Neurosci 107(3), 373-381 (2001); herein incorporated by reference), mast cells (Nozawa, et al., Neurosci Lett. 309, 33-36 (2001); herein incorporated by reference), pulmonary arterial and aortic smooth muscle (Biró, et al., Blood 91, 1332-1340 (1998); herein incorporated by reference and spleen (Yang, et al., Am J Physiol Lung Cell Mol Physiol 290(6), L1267-1276 (2005); herein incorporated by reference). The rapidly growing list of human tissues that express TRPV1 currently includes human epidermal keratinocytes, (Denda, et al., Biochem Biophys Res Commun 285, 1250-1252 (2001); Inoue, et al., Biochem Biophys Res Commun 291, 124-129 (2002); herein incorporated by reference), mast cells (Ständer, et al., Exp Dermatol 13, 129-139 (2004); herein incorporated by reference), epithelial cells of hair follicle (Inoue, et al., Biochem Biophys Res Commun 291, 124-129 (2002); herein incorporated by reference), sweat glands (Inoue, et al., Biochem Biophys Res Commun 291, 124-129 (2002); herein incorporated by reference), sebaceous glands (Inoue, et al., Biochem Biophys Res Commun 291, 124-129 (2002); herein incorporated by reference), bladder urothelium (Apostolidis, et al., Urology 65, 400-405, (2005); herein incorporated by reference), kidney (Cortright, et al., Biochem and Biophys Res Comm 281, 1183-1189 (2001); herein incorporated by reference), cerebral cortex (Cortright, et al., Biochem and Biophys Res Comm 281, 1183-1189 (2001); herein incorporated by reference), cerebellum (Cortright, et al., Biochem and Biophys Res Comm 281, 1183-1189 (2001); herein incorporated by reference), and hypothalamus (Mezey, et al., Proc. Natl. Acad. Sci. USA 97, 3655-3660 (2000); herein incorporated by reference), among others. Thus TRPV1 was widely implicated in physiology and pathology, as comprehensively reviewed elsewhere glands (Nagy et al., Eur J Pharmacol 500, 351-369 (2004); Caterina, Pain 105, 5-9 (2003); herein incorporated by reference).

III. Agonists and Antagonists of TRPV1.

TRPV1's wide expression suggests that it has a complex mode of activation and regulation, and indeed this is the case. Consistent with the suggestion that TRPV1 is a polymodal integrator of noxious stimuli, numerous biological and synthetic compounds were discovered that can activate the receptor. Three categories of TRPV1 activation of use in the present invention include: receptor activation, ligand activation, and direct activation (Ramsey, M et al., Annu Rev Physiol 68, 619-647 (2006); herein incorporated by reference). Receptor activation refers to the activation of isoforms of phospholipase C through the activity of G-protein coupled receptors and receptor tyrosine-kinases. These TRPV1 cell activities result in phosphotidylinositol-4,5-bisphosphate (PIP2) hydrolyzed into diacylglycerol and inositol-3,4,5-trisphosphate (IP3), products which can modulate TRPV1 function. Furthermore, specific isoforms of PKA and PKC are activated that can modulate the channel's status through phosphorylation events. Ligand activation, the most commonly studied form of activation of TRPV1 proteins, refers to the binding of either exogenous or endogenous small organic molecules, inorganic ions (such as H+), or products of lipid or nucleotide metabolism to the channel (either extra- or intra-cellularly) in a way that causes a conformational change in the channel that opens the pore to allow influx of cations.

Finally, direct activation refers to mechanical stimuli or changes in temperature. TRPV1 was the founding member of the vanilloid subfamily of TRP subunits because they are activated by molecules with a vanillyl moiety, including capsaicin, olvanil, and others. Capsaicin and anandamide (arachidonoyl ethanolamide), a vanilloid-like compound that is a potent agonist at the TRPV1 receptor (Ross, Brit J Pharmacol 140, 790-801 (2003); herein incorporated by reference), have been most widely studied and used in the biochemical and pharmacological characterization of the receptor.

Anandamide was discovered as an agonist of the TRPV1 receptor when patch-clamp experiments expressing TRPV1 exhibited anandamide-induced currents in whole cells and isolated membrane patches (Zygmunt, et al., Nature 400, 452-456 (1999); herein incorporated by reference). Anandamide was later described as a full agonist at the human TRPV1 receptor when electrophysiological experiments showed that both capsaicin and anandamide induced similar Ca2+-mediated inward currents in hTRPV1 transfected HEK293 cells (Smart, et al., Brit J Pharmacol 129, 227-230 (2000); herein incorporated by reference). Later it was shown that anandamide is restricted in producing a Ca2+-mediated current in primary cultures of DRG neurons at a pH<6.5 (Olah, et al., J Biol Chem 276(33), 31163-31170 (2001); herein incorporated by reference). Much interest has surrounded anandamide as a potential candidate for the endogenous activator of TRPV1 proteins, but this has not yet been shown. A recent study provides evidence that anandamide is formed in cells following activation of the PLC/IP3 pathway by a rise in intracellular calcium (Van der Stelt, et al., Eur Mol Bio Org Jour 24, 3026-3037 (2005); herein incorporated by reference). The inventor's contemplate that anandamide could function as a second messenger inside the cell that amplifies calcium levels via TRPV1 by sensing the calcium release from intracellular stores

Other ligands were identified and synthesized that activate TRPV1 that may find use in methods of treatment of the present inventions. These include but are not limited to methanandamide (Malinowska, et al., Naunyn-Schmiedeberg's Arch Pharmacol 364, 562-569 (2001); Ralevic, et al., Brit J Pharmacol 130, 1483-1488 (2000); herein incorporated by reference), N-arachidonoyl-dopamine (NADA), (Huang, et al., J Neurophysiol 95, 1207-1212 (2006); Huang, et al., Proc Natl Acad Sci USA 99(12), 8400-8405 (2002); herein incorporated by reference), resiniferatoxin (RTX), rinvanil and its derivatives (Huang, et al., Proc Natl Acad Sci USA 99(12), 8400-8405 (2002); herein incorporated by reference), 2-aminoethoxydiphenyl borate (2-APB), (Hu, et al., J Biol Chem 279(34), 35741-35748 (2004); Gu, et al., AJP-Lung 288, 932-941 (2005); herein incorporated by reference), the peripheral satiety factor oleoylethanolamide (Ahern, J Biol Chem 278(33), 30429-30434 (2003); herein incorporated by reference), N-Oleoyldopamine Chu, et al., J Biol Chem 278(16), 13633-13639 (2003); herein incorporated by reference), ethanol (Trevisani, et al., Nature Neurosci 5(6), 546-551 (2002); Gazzieri, et al., Cardiovasc Res 70, 589-599 (2006); Trevisani, et al., Nature Neurosci 5(6), 546-551 (2002); herein incorporated by reference), as well as several lipoxygenase products (Hwang, et al., Proc Natl Acad Sci USA 97(11), 6155-6160 (2000); herein incorporated by reference), such as 12-(S)-HPETE, 15-(S)-HPETE, 5-(S)-HETE, 15-(S)-HETE, leukotriene B4, and a cytochrome P450 product 20-HETE (Scotland, et al., Circ Res 95, 1027-1034 (2004); herein incorporated by reference).

Similarly, ATP was shown to potentiate TRPV1 activity through its interaction with P2Y receptors in a PKC-dependent pathway (Tominaga, et al., Proc Natl Acad Sci USA 98(12), 6951-6956 (2001); herein incorporated by reference). 1,2-Napthoquinone causes the phosphorylation of protein tyrosine kinases, leading to the activation of the phospholipase A2/lipoxygenase signaling pathway that causes the TRPV1-dependent contraction of guinea pig tracheal smooth muscle (Kikuno, et al., Tox Appl Pharmacol 210, 47-54 (2006); herein incorporated by reference).

Similarly, several compounds have been identified that antagonize TRPV1 receptor activation that may find use for impairing TRPV1 for identifying TRPV1 specific therapeutic drugs. These inhibitors include but are not limited to capsazepine and ruthenium red (RR) (Wardle, et al., Br J Pharmacol 121, 1012-1016 (1997); herein incorporated by reference), a high-affinity iodo-resiniferatoxin (Rigoni, et al., Brit J Pharmacol 138, 977-985 (2003); herein incorporated by reference), thiazole carboxamides (Xi, et al., Bioorg Med Chem Ltrs 15, 5211-5217 (2005); herein incorporated by reference), A-425619 (Kouhen, et al., J Pharmacol Experim Therap 314(1), 400-409 (2005); McGaraughty, et al., J Neurophysiol 95, 18-25 (2006); herein incorporated by reference), AMG 9810 (Xi, et al., Bioorg Med Chem Ltrs 15, 5211-5217 (2005); herein incorporated by reference), and N-Acylvanillamines such as arvanil (Marquez, et al., Mol Pharmacol 69(4), 1373-1382 (2006); herein incorporated by reference). Together these compounds represent a class of compounds that are known to inhibit TRPV1 channel activation. TRPV1 antagonists have come under intense investigation due to their therapeutic promise in the treatment of pain (Szallasi, et al., J Med Chem 47(11), 2717-2723 (2004); herein incorporated by reference). Thus much effort has gone into understanding the molecular determinants of receptor agonism and antagonism as exhibited by the various modulators of TRPV1 function.

The discovery that vanilloids bind to TRPV1 from the intracellular side led investigators to develop deletion and site-directed mutagenesis experiments on the cytosolic tails of both termini to determine ligand recognition sites. Such experiments determined that Glu-761 and Arg-114 are recognition sites for capsaicin binding in rat TRPV1 (Jung, et al., J Biol Chem 277(46), 44448-44454 (2002); herein incorporated by reference). When these two mutants were coexpressed, capsaicin elicited no inward current, indicating that vanilloids interact with both termini of the TRPV1 subunit. Gating by capsaicin is notably more complex, however, as indicated by the potentiation of channel opening by heat and acid. A molecular determinant for the potentiation of capsaicin activation by acid was localized to Glu-600 (Jordt, et al., Proc Natl Acad Sci USA 97(14), 8134-8139 (2000); herein incorporated by reference). Jordt, et al., also demonstrated that acid was enough to activate the receptor itself in a way distinct from other forms of activation, as mutations at Glu-648 selectively destroyed proton-evoked activation without affecting channel responses to vanilloids or heat.

In addition to TRPV1's complex interaction with vanilloid ligands, it was reported that distinct mechanisms exist for TRPV1 activation by either heat or acid (Welch, et al., Proc Natl Acad Sci USA 97(25), 13889-13894 (2000); herein incorporated by reference). A potent TRPV1 antagonist iodoresiniferatoxin (IRTX) prevented heat-evoked responses in rats (Jhaveri, et al., Eur J Neurosci 22(2), 361-70 (2005); herein incorporated by reference). Understanding how agonists and antagonists interact with the receptor is an important area of research for those interested in drug design.

A study that utilized rabbit TRPV1 identified key residues in transmembrane regions 3 and 4 that are critical for vanilloid binding. Mutations in Met-547 and Thr-550 were able to confer vanilloid sensitivity (both capsaicin and RTX) to rabbit TRPV1 that is normally 100-fold less sensitive to vanilloids than either rat or human (Jung, et al., J Biol Chem 277(46), 44448-44454 (2002); herein incorporated by reference). These findings add to the previously reported finding that Tyr-511 and Ser-512 are critical residues for conferring vanilloid sensitivity in TRPV1 (Jordt, et al., Proc Natl Acad Sci USA 97(14), 8134-8139 (2000); herein incorporated by reference). Another study found that mutating the Ser-512 equivalent residue in human TRPV1 destroyed vanilloid-dependent activation of the channel even though capsaicin was still able to interact with the mutant residue (Gavva, et al., J Biol Chem 279(19), 20283-20295 (2004); herein incorporated by reference). Thus it is evident that several distinct areas of the channel are involved in its gating by vanilloid ligands and of use for impaired TRPV1 receptors of the present inventions (For review, see, Ferrer-Montiel, et al., Eur J Biochem 271, 1820-1826 (2004); herein incorporated by reference).

IV. TRPV1 Signaling Pathways.

Beyond regulation at the gate of the channel, TRPV1 is known to be regulated by a variety of intracellular signaling pathways. The actions of several protein kinases and phosphatases work in concert to determine the activation status of the channel. These and other mechanisms of TRPV1 regulation will now be briefly reviewed.

In 2000 it was discovered that TRPV1 channel activity could be induced by the activation of protein kinase C(PKC), and that bradykinin and anandamide enhanced channel activity in a PKC-dependent manner (Premkumar, et al., Nature 408, 985-990 (2000); herein incorporated by reference). PKC has also been implicated in re-sensitization of the receptor after desensitization was induced by repeated capsaicin treatment (Mandadi, et al., Cell Calcium 35, 471-478 (2004); herein incorporated by reference). Later studies used the phorbol ester PMA (an activator of intracellular PKC) to identify Ser-502 and Ser-800 as key target residues of PKCε phosphorylation on rat TRPV1's first intracellular loop and C-terminus, respectively (Numazaki, et al., J Biol Chem 277(16), 13375-13378 (2002); herein incorporated by reference).

These results were expanded upon in a Bhave, et al. study in which in vitro phosphorylation and protein sequencing techniques were used to add Thr-704 from the C-terminus and Thr-144 from the N-terminus to the list of major PKC phosphorylation sites (Bhave, et al., Proc Natl Acad Sci USA 100(21), 12480-12485 (2003); herein incorporated by reference). PKC's role in receptor desensitization is not limited to phosphorylation status, however. Deletion studies of rat TRPV1 have indicated that the distal C-terminus plays an inhibitory role in counteracting PKC stimulation (Liu, et al., J Physiol 560(3), 627-638 (2004); herein incorporated by reference).

Knowledge of the role of PKC on TRPV1 activation led to the search for ways in which PKC could be activated in the physiologic state, particularly during inflammation. Chuang, et al. suggested that both of the inflammatory mediators bradykinin and nerve growth factor (NGF) modulate TRPV1 sensitivity through both PKC-dependent and independent pathways (Bhave, et al., Proc Natl Acad Sci USA 100(21), 12480-12485 (2003); herein incorporated by reference). Both bradykinin and NGF stimulate phospholipase C to cleave PIP2 into IP3 and DAG. Consequences of this event are two-fold. First IP3 causes the release of calcium from intracellular stores. Increased intracellular calcium then works in concert with DAG to activate PKC, which can subsequently stimulate TRPV1.

Furthermore, a more recent study indicated that PLC-dependent potentiation of TRPV1 is dependent upon residues 777-820, a PIP2 binding site on the C-terminus of the receptor (Prescott, et al., Science 300, 1284-1288 (2003); herein incorporated by reference). Thus activation of PLC can relieve TRPV1 from PIP2-mediate inhibition. In addition to its role in directly mediating receptor activity, PKC was implicated in the trafficking of vesicular channels containing TRPV1 to the cell surface via SNARE-dependent exocytosis (Morenilla-Palao, et al., J Biol Chem 279(24), 25665-25672 (2004); herein incorporated by reference). These authors suggested that this works through the rapid recruitment of the SNARE proteins Snapin and Syt IX, two proteins identified to interact with the ankyrin repeats on the N-terminus of TRPV1. This represents a form of indirect regulation that can sensitize or desensitize the channel to stimulation by a specific agonist molecule (Ramsey, M et al., Annu Rev Physiol 68, 619-647 (2006); herein incorporated by reference).

These and other studies indicated that PKC signaling could be a mechanism for transducing environmental stimuli into TRPV1 activation. The activation of TRPV1 via PKC-dependent pathways provides a mechanism through which inflammatory mediators such as nerve growth factor, bradykinin, and the proinflammatory cytokine IL-1β can contribute to TRPV1-mediated inflammatory hyperalgesia (Tang, et al., Eur J Pharmacol 498, 37-43 (2004); Obreja, et al., FASEB J 16, 1497-1503 (2002); herein incorporated by reference). Future studies can be expected to implicate other endogenous inflammatory mediators in this TRPV1-mediated pathway.

PKC is one of several intracellular kinases involved in modulation of TRPV1 activity. Examples of such kinases are prostaglandin E2, a compound that enhanced the gating of TRPV1 channels stimulated by capsaicin mediated via the cAMP-PKA signaling pathway (Lopshire, et al., J Neurosci 18(16), 6081-6092 (1998); herein incorporated by reference) and PKA that inhibited the capsaicin-evoked desensitization of the channel by phosphorylating Ser-116 and Thr-370 residues (Mohapatra, et al., J Biol Chem 278(50), 50080-50090 (2003); herein incorporated by reference). In addition, Ser-502 was discovered as a PKA phosphorylation site (Rathee et al., J. Neurosci. 22(11), 4740-4745 (2002); herein incorporated by reference). The importance of PKA in this event is affirmed by studies that inhibited calcineurin, a phosphatase found to regulate the desensitization of the capsaicin-activated channel. (Docherty, et al., Flugers Arch-Eur J Physiol 431, 828-837 (1996); herein incorporated by reference). When the cyclosporine A•cyclophilin A complex, a specific inhibitor of calcineurin, was applied intracellularly, a decrease in capsaicin-mediated desensitization was observed similar to when intracellular PKA was activated (Mohapatra, et al., J Biol Chem 280(14), 13424-13432 (2005); herein incorporated by reference).

Also regulating the capsaicin-mediated calcium-dependent desensitization is the Ca2+-calmodulin dependent kinase II (CaMKII). In 2004 a study found that mutations at two consensus sites for this kinase failed to confer a capsaicin-sensitive current (Jung, et al., J Biol Chem 279(8), 7048-7054 (2004); herein incorporated by reference). Phosphorylation by PKA and PKC converge at these same sites. Together these results indicate that PKA, PKC, and CaMKII work in opposition to calcineurin to regulate the calcium-dependent desensitization of TRPV1.

This aforementioned regulation of TRPV1 by phosphorylation and dephosphorylation is one of several physiologically relevant methods of regulation. Several TRPV1 splice variants isolated in mouse and rat models that can modulate TRPV1-mediated responses to environmental stimuli. A mouse variant TRPV1β encodes a dominant-negative subunit of the TRPV1 channel such that it inhibits channel activity when it associates with other normal TRPV1 subunits in a tetramer. Wang, et al., J Biol Chem 279(36), 37423-37430 (2004); herein incorporated by reference. Furthermore, a novel splice variant was recently discovered in the rat renal papilla that may be an additional point of regulation to the TRPV1-mediated response to a salt load. Tian, et al., AJP-Renal 290, 117-126 (2006); herein incorporated by reference.

Other noteworthy regulation of TRPV1 function includes the increased expression of TRPV1 receptors as mediated by the inflammatory mediators ATP, bradykinin, and NGF (Amaya, K et al., Brain Res 963(1-2), 190-196 (2003); herein incorporated by reference) regulation of mean open and closed channel times by the association of Fas-associated factor 1 (FAF1) with the N-terminus of TRPV1 (Kim, et al., J Neurosci 26(9), 2403-2412 (2006); herein incorporated by reference), the mobilization of TRPV1 to the plasma membrane in the presence of insulin (Van Buren, et al., Mol Pain 1, 17 (2005); herein incorporated by reference), the regulation of the oxidation state of key cysteine residues on the extracellular side of the receptor by dithiothreitol (Vyklicky, et al., Neuroscience 111(3), 435-441 (2002); herein incorporated by reference), and the upregulation of mRNA expression by neurotrophic factors.35 As a result of this complex web of regulatory mechanisms the inventors contemplate the use of these systems for designing drugs for use in many physiological and pathophysiological states, in particular for pathological conditions.

V. The Function of TRPV1 as a Chemo- and Mechano-Sensor.

The polymodal activation of TRPV1 by such noxious stimuli as high temperatures and acidity have indicated that one of its major functions is to act as a molecular transducer of a painful physico-chemical environment. Beyond nociception, the role of TRPV1 has expanded to other aspects of physiological regulation, notably the cardiovascular system. A prominent example involves the role of TRPV1 in the antihypertensive mechanisms induced by sodium loading (Wang, et al., Hypertension 33(15), 499-503 (1999); herein incorporated by reference). In one embodiment, the inventors contemplate that the release of the vasodilatory neuropeptides substance P(SP) and/or calcitonin gene-related peptide (CGRP) from terminals of sensory neurons innervating the vascular beds compensates for a rise in systemic blood pressure. (Supowit, et al., AJP-Heart 289, 1169-1175 (2005); herein incorporated by reference). It is currently unknown, however, as to how rises in plasma/interstitial sodium concentrations or intralumenal pressure lead to activation of TRPV1 in vivo. Thus it is of great interest to understand how the receptor senses the chemical and mechanical environment in the vasculature and kidney and integrates altered environmental stimuli to lead to compensatory responses in a pathophysiological state such as hypertension.

TRPV1 acts as a sensor of a wide range of chemical stimuli. The question remains, however, as to the identity of the endogenous ligands of TRPV1 in the CNS and peripheral sites. At least three to four categories of endogenous candidates were suggested as anandamide, lipoxygenase and cytochrome P450 products of arachidonic acid, and N-arachidonoyldopamine (Scotland, et al., Circ Res 95, 1027-1034 (2004); van der Stelt, et al., Eur J Biochem 271, 1827-1834 (2004); herein incorporated by reference). While much effort was put forth to understand the biosynthesis and degradation of each of these possible endogenous ligands, little data exists on the role of each in activating TRPV1 in vivo. Thus future studies will be focused on determining the roles of each in the pathophysiological state, a process that the inventor's believe will lead to the discovery of new candidates for endogenous activation of TRPV1.

Evidence regarding the role of TRPV1 as a mechanosensor is at best, inconclusive. No evidence is available supporting the direct activation of TRPV1 via sheer stress generated by the flow in the lumen of blood vessels. However, TRPV1 may be activated indirectly by altered transmural pressure through the production of 20-hydroxyeicosatetraenoic acid (20-HETE), a known activator of TRPV1 that causes the release of SP when it binds to the TRPV1 expressed on sensory C-fibers. The same authors concluded that TRPV1 was not a mechanosensor, however, because the stretch-activated nonselective cation channel blocker gadolinium had no effect on capsaicin-induced activation of TRPV1. On the contrary, indirect evidence indicates that perhaps TRPV1 is a mechanosensor. As a result of the subsequent increase in cellular volume, an osmotic stimulus leads to changes in membrane tension that can be regarded as a mechanical stimulus. When TRPV1 null mice were studied it was found that the bladder epithelial cells did not respond to a hypotonic osmotic stimulus (Birder, et al., Nature Neuroscience 5(9), 856-860 (2002); herein incorporated by reference). Wild type bladder urothelial cells, however, responded to a hypotonic stimulus with a release of ATP. Thus the authors concluded that the absence of TRPV1 in the urothelium inhibits normal bladder function and the normal mechanically evoked purinergic signaling by the urothelium. It may also be possible that TRPV1 coassembles with distinct mechanosensitive TRPV subtypes or other proteins in the bladder (Kobayashi, et al., J Comp Neurol 493, 596-606 (2005); herein incorporated by reference).

A study by Naeini, et al. implicates TRPV1 in the osmosensory transduction mediated by arginine-vasopressin (AVP)-releasing neurons in the supraoptic nucleus (SON) of the hypothalamus (Naeini, et al., Nature Neurosci 9(1), 93-98 (2006); herein incorporated by reference). In this study it was shown that N-terminal splice variants of TRPV1 are expressed by AVP neurons in the SON. In a TRPV1-positive animal, these cells would shrink in response to a hyperosmotic stimulus, activating a stretch-inhibited osmosensory transduction channel (Oliet, et al., Nature 364, 341-343 (1993); herein incorporated by reference). This would lead to depolarization that contributes to cellular excitation and AVP release. TRPV1 knockout mice were unable to respond to hyperosmotic stimulation, indicating that the TRPV1 gene may encode a central component of the osmoreceptor that regulates systemic levels of AVP.

VI. The Role of TRPV1 in the Regulation of Salt and Water Homeostasis in the Physiologic State.

The aforementioned study by Naeini, et al. indicates that a TRPV1 splice variant in the central nervous system plays an important role in antidiuresis in response to serum hyperosmolality (Naeini, et al., Nature Neurosci 9(1), 93-98 (2006); herein incorporated by reference), which appears to be opposite to the peripheral response in which TRPV1 seems to be activated by the hypotonic stimulus (Birder, et al., Nature Neuroscience 5(9), 856-860 (2002); herein incorporated by reference). Thus it seems apparent that TRPV1 is an important player in the regulation of salt and water homeostasis in the physiologic state. The evidence supporting such a role for TRPV1 is shown below. In addition to their sensory afferent function, TRPV1-positive neurons also have an efferent motor function. Binding of agonists to TRPV1 causes the opening of the cation channel that leads to the influx of sodium and calcium ions (Winter, et al., Brain Res. 520, 13-40 (1990); Bevan, et al., J. Physiol. 398, 28 (1988); Forbes, et al., Neurosci. Lett. S3, 32 (1998); Marsh, et al., Neuroscience, 23, 275-289 (1987); Wood, et al., J. Neurosci. 8, 3208-3220 (1988); all of which are herein incorporated by reference). While sodium influx is high enough to depolarize the neuron and lead to an afferent impulse conduction, calcium influx is required for the release of neuropeptides from sensory nerve endings (Maggi, The pharmacological modulation of neurotransmitter release, in: Capsaicin in the study of pain, edited by J. Wood (Harcourt Brace & Company, New York, 1993), pp. 161-189). At least 12 different types of transmitters have been identified in TRPV1-positive sensory neurons that can be released alone or with other peptides. These neuropeptides include SP, neurokinin A, neuropeptide K, eledoisin-like peptide, somatostatin, vasoactive intestinal polypeptide, cholecystokinin-octapeptide, CGRP, galanin, corticotrophin-releasing factor, arginine vasopressin, and bombesin-like peptides (Maggi, The pharmacological modulation of neurotransmitter release, in: Capsaicin in the study of pain, edited by J. Wood (Harcourt Brace & Company, New York, 1993), pp. 161-189).

The vasodilatory neuropeptides SP and CGRP have been shown to be extremely relevant to the discussion of whole body salt and water homeostasis. For example, they have been shown to have direct and indirect effects on tubular ion transport in the kidney, resulting in natriuretic and diuretic actions. Wimalawansa, Endocr. Rev. 17, 533-585 (1996); herein incorporated by reference; Arendshorst, et al., Am. J. Physiol. 230, 1662-1667 (1976); herein incorporated by reference; Shekhar, et al., Am. J. Cardiol. 67, 732-736 (1991); herein incorporated by reference. Furthermore, CGRP and SP are often co-localized within the nerve endings of sensory nerves found around blood vessels in the majority of vascular beds (Wimalawansa, Endocr. Rev. 17, 533-585 (1996); herein incorporated by reference; Breimer, et al., Biochem. J. 255, 377-390 (1988); herein incorporated by reference; Preibisz, Am. J. Hypertens. 6, 434-450 (1993); herein incorporated by reference; Zaidi, et al., Crit. Rev. Clin. Lab. Sci. 28, 109 (1999); herein incorporated by reference; McEwan, et al., Calcitonin gene-related peptide: a review of its biology and relevance to the cardiovascular system, in: Endocrine mechanisms in hypertension, edited by Laragh, et al., (Raven Press, New York, 1989), p. 287); herein incorporated by reference). This raises the possibility that TRPV1 receptors located in sensory nerves innervating the vascular beds sense local and/or systemic chemical (e.g., changes in concentrations of ions or endocrine/paracrine/autocrine factors) or mechanical (e.g. changes in transmural pressure) stimuli and release neuropeptides such as SP and CGRP to mediate a response.

In addition, the TRPV1 agonist capsaicin was shown to be a selective toxin for sensory neurons. Capsaicin binds to TRPV1 receptors on DRG neurons causing downregulation of the receptor, depletion of sensory neurotransmitters, and sensory nerve degeneration when given to neonates or in high doses to adults (Szallasi, et al., Pharmacol. Rev. 51, 159-210 (1999); herein incorporated by reference). The inventors found that degeneration of sensory nerves causes a subsequent rise in systemic blood pressure in rats challenged with a salt load (Wang, et al., Hypertension 33(15), 499-503 (1999); Wang, et al., Hypertension 37, 440-443 (2001); Wang, et al., Hypertension (Rapid Communication) 32(4), 649-653 (1998); Wang, et al., J Hypertension 21(2), 403-409 (2003); Li, et al., J Hypertension 21(3), 577-582 (2003); all of which are herein incorporated by reference). This implied that TRPV1 normally functions to increase natriuresis and diuresis in response to a salt challenge.

Furthermore, the inventors discovered that TRPV1 present in a subpopulation of sensory nerves innervating the kidney is responsible for capsaicin-induced bilateral increases in diuresis and natriuresis (Zhu, et al., Hypertension 46(part 2), 992-997 (2005); herein incorporated by reference). In this experiment capsaicin was administered either into the unilateral renal pelvis or intravenously in the presence or absence of a selective TRPV1 antagonist or unilateral renal denervation. Increases in Uflow and UNa were observed in a dose-dependent fashion that could be abolished through blockade of TRPV1 or by ipsilateral renal denervation. Thus activation of TRPV1 in the unilateral renal pelvis resulted in an enhanced bilateral renal function via the renorenal reflex.

VII. The Role of TRPV1 in the Regulation of Salt Sensitivity of Arterial Pressure.

The previously reviewed studies indicate that TRPV1 is widely expressed in the body in areas that have a direct effect on salt and water homeostasis, notably the osmosensory neurons in the CNS, sensory nerves lining the blood vessels, and sensory nerves innervating the kidney. In combination it is likely that such expression in the body indicates a central role for TRPV1 in the regulation of blood pressure homeostasis. Understanding the interactions between blood volume and sodium levels, TRPV1, the renin-angiotensin-aldosterone system (RAAS), the sympathetic nervous system, the endothelin system, and increased oxidative stress have obvious implications with regards to the treatment of hypertension. Several studies have investigated the importance of TRPV1 with regard to hypertension, particularly in models of salt sensitivity.

The inventor's demonstrated for the first time in 1998 that neonatal degeneration of TRPV1-positive sensory nerves rendered an adult rat salt-sensitive (Wang, et al., Hypertension (Rapid Communication) 32(4), 649-653 (1998); herein incorporated by reference). As a result rats loaded with salt showed an impaired natriuretic and diuretic response along with an increase in blood pressure. To determine an interaction between the sensory nervous system and the RAAS or sympathetic nervous system, either losartan (a type I angiotensin II receptor blocker), prazosin (a selective α1-adeno-receptor blocker), or hydralazine (a nonspecific vasodilator) was administered to high-salt plus capsaicin pretreated rats. Wang, et al., Hypertension 33(15), 499-503 (1999); herein incorporated by reference. Losartan and hydralazine, but not prazosin, prevented the development of salt-induced hypertension in this model. This implies that in sensory intact rats there is an interaction between the sensory nervous system and the RAAS in preventing the development of salt-induced hypertension. However, because losartan and hydralazine were unable to prevent the impaired natriuretic response, it suggests that intact sensory innervation is essential for a normal natriuretic response regardless of blood pressure. It is likely that losartan and hydralazine work by vasodilatory mechanisms and not by protecting against the impaired sodium excretion in the model.

In a study designed during the development of the present inventions to understand the molecular mechanisms behind the salt activation of sensory nerves, the inventors tested the effects of capsaicin and capsazepine on rats fed either a high or normal sodium diet. Li, et al., J Hypertens 21, 1525-1530 (2003); herein incorporated by reference. Several important findings can be taken from this study. First it was found that activation of TRPV1 by capsaicin led to an increase in plasma CGRP levels and a decrease in MAP because these actions were blocked by capsazepine. Secondly, when capsazepine was administered to rats fed a high salt diet, the pro-hypertensive effects of salt were unmasked. Furthermore, western blot analysis indicated that TRPV1 receptor expression increased in both mesenteric resistance arteries and the renal medulla in response to a high salt diet. Finally, it was shown using immunohistochemical techniques that at least a subset of TRPV1-positive sensory neurons contains CGRP because TRPV1 and CGRP co-localized in the perivascular sensory nerves innvervating the mesenteric resistance arteries. The inventor's hypothesized that the upregulation in the renal medulla could mediate an increase in the release of CGRP that would bind in a paracrine fashion to its receptors in the renal medulla where it could affect salt and water homeostasis.

The inventors recently discovered that the depressor effect mediated by anandamide was prevented when both TRPV1 and CB1 receptors were blocked (Wang, et al., Hypertension 46(part 2), 986-991 (2005); herein incorporated by reference). Thus TRPV1 at least partially contributes to the decrease in peripheral vascular resistance caused by anandamide-induced release of 'CGRP, a mechanism that also operates in spontaneously hypertensive rats (Li, et al., Hypertension 41(2), 757-762 (2003); herein incorporated by reference). Furthermore, the inventors found from the study of Dahl salt sensitive rats that a lack of TRPV1 receptor activity in these genetically predisposed rats eliminated the normal counterregulatory action of sensory nerves, leaving these rats without protection in the face of a salt challenge (Wang, et al., Hypertension 47 (part 2), 609-614 (2006); herein incorporated by reference). These results have led us to develop the hypothesis that TRPV1 expressed in a genetically distinct subpopulation of primary sensory nerves is activated in response to high salt intake, which promotes natriuresis and prevents salt-induced increases in blood pressure via counterbalancing the prohypertensive systems.

The contemplated hypothesis of the inventors is described as follows. Activation of TRPV1 by mechanical and chemical stimuli results in the release of CGRP and SP which promotes natriuresis and diuresis through their actions on the kidney. TRPV1 also affects kidney function via descending pathways from the CNS.

While it is certainly an attractive hypothesis that TRPV1-induced release of CGRP normally maintains blood pressure through its local effects on peripheral resistance and its effects on tubular ion transport in the kidney, the picture is most certainly more complicated. An interaction between the sensory nervous system and the RAAS has already been discussed (Wang, et al., Hypertension 33(15), 499-503 (1999); herein incorporated by reference; Huang et al., J Hypertension 19(10), 1841-1846 (2001); herein incorporated by reference; Huang, et al., Am J Physiol (Heart & Circulatory Physiology) 281 (5), H2143-H2149 (2001); herein incorporated by reference).

Furthermore, it is known that the sympathetic nervous system, (Wang, et al., Hypertension 37, 440-443 (2001); herein incorporated by reference), the endothelin system (Ye, et al., Hypertension 39 (2 pt 2), 673-678 (2002); herein incorporated by reference; Wang et al., Am J Physiol (Heart & Circulatory Physiology) 289, H2005-H2011 (2005); herein incorporated by reference), and oxidative stress generation Song, et al., Act Pharmacol Sin 25(12), 1626-1632 (2004); herein incorporated by reference) becomes more active upon sensory nerve degeneration plus high salt intake. The role of TRPV1 and how it interacts with these systems to prevent hypertension is comprehensively reviewed elsewhere (Wang Acta Pharmacologica Sinica 26(3), 286-294 (2005); herein incorporated by reference).

VIII. TRPV1 as a Target for Future Drug Development.

The wide expression of TRPV1 in the body indicates that agonism and antagonism of the receptor has enormous therapeutic potential. Possible therapeutic interventions may include the use of selective blockers, down-regulation strategies such as antisense treatment, or desensitization of TRPV1 (Nilius, et al., Sci STKE 2005(295), re8, 2 (2005); herein incorporated by reference). One of the most widely studied of these possibilities is the use of TRPV1 antagonists in the treatment of pain (Szallasi, et al., J Med Chem 47(11), 2717-2723 (2004); herein incorporated by reference). Furthermore, the fact that agonists of TRPV1 are able to induce a CGRP-mediated hypotensive effect in salt hypertensive rats indicates that activating TRPV1 may be an effective means of preventing the development of hypertension (Wang et al., Am J Physiol (Heart & Circulatory Physiology) 289, H2005-H2011 (2005); herein incorporated by reference), and its associated end organ damage. Indeed, the inventor's showed that a TRPV1 gene knockout impairs postischemic recovery in isolated perfused heart in mice (Wang, et al., Circulation 112(23), 3617-3623 (2005); herein incorporated by reference), indicating that TRPV1 plays a protective role against ischemic injury of the heart. This in itself is an exciting possibility considering that an estimated 65 million Americans and 1 billion subjects worldwide suffer from hypertension and its associated end organ damage review (Nagy et al., Eur J Pharmacol 500, 351-369 (2004); herein incorporated by reference).

The quest to find safe, effective therapeutics to act primarily on TRPV1 is currently hindered by the lack of endogenous ligands or synthetic ligands of the receptor for use in clinical treatments. Understanding the endogenous activation of TRPV1 as well as its expression, regulation, and post-signaling pathways are necessary in order to understand, identify therapeutic drugs, and treat pathologies related to cardiovascular homeostasis, hypertension, and end organ damage that results from uninhibited pathological conditions.

In a preferred embodiment, the present invention relates to using a 20-hydroxyeicosatetraenoic acid (20-HETE) analog, DSR-II-247-30: 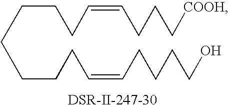
for treating hypertension patients, in particular salt sensitive hypertension patients.

In one embodiment the following compound: 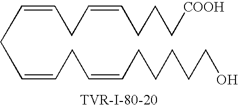
is contemplated for treating hypertension patients, in particular salt sensitive hypertension patients.

In one embodiment the following compound: 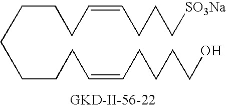
is contemplated for treating hypertension patients, in particular salt sensitive hypertension patients.

In one embodiment, the following compound:

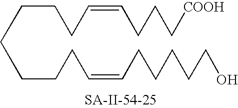

is contemplated for treating hypertension patients, in particular salt sensitive hypertension patients.

TABLE 1
Mean arterial pressure before and after treatments (mmHg)

| | Baseline | Treatment |
|---|---|---|
| | mmHg | mmHg |
| Vehicle | 119 ± 4 | 118 ± 3 |
| 600 mM NaCl | 115 ± 2 | 117 ± 2 |
| CAPZ + NaCl | 120 ± 5 | 123 ± 5 |
| RD + NaCl | 122 ± 5 | 122 ± 4 |
| RP67580 + NaCl | 124 ± 3 | 125 ± 3 |
| 1200 mOsm Mannitol | 121 ± 6 | 121 ± 7 |
| CAPZ + Mannitol | 124 ± 7 | 123 ± 5 |
| 150 KCl | 123 ± 6 | 125 ± 4 |
| CAPZ + KCl | 121 ± 7 | 121 ± 5 |

Values are expressed as mean ± SE, n = 5-6 rats in each group.

### EXPERIMENTAL

The following examples serve to illustrate certain embodiments and aspects of the present invention and are not to be construed as liming the scope thereof. In the experimental disclosures which follow, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); pg (picograms); L and l (liters); ml (milliliters); μl (microliters); cm (centimeters); mm (millimeters); μm (micrometers); nm (nanometers); U (units); min (minute); s and sec (second); k (kilometer); deg (degree); ° C. (degrees Centigrade/Celsius).

The following examples demonstrate therapeutic compound induced activation of the TRPV1 receptors for lowering salt induced high blood pressure (mean arteriole pressures), loss of therapeutic activity in TRPV1 impaired animals, tissues and cells, and efficacy of therapeutic compounds while impairing CB receptors. Additionally, embodiments demonstrating drug screening using whole animals, tissues, and cells, are shown for heart, kidney, sensory ganglia et cetera.

In addition the teachings described herein, the following publications are herein, incorporated by reference in their entirety: VR1-Mediated Depressor Effects During High-Salt Intake Role of Anandamide (Wang et al., Hypertension. 2005 October; 46(4):986-91. Epub 2005 September 6); Diuresis and Natriuresis Caused by Activation of VR1—Positive Sensory Nerves in Renal Pelvis of Rats Hypertension (Zhu et al., 2005 October; 46(4):992-7. Epub 2005 August 8); Enhanced oxidative stress in kidneys of salt-sensitive hypertension: role of sensory nerves (Wang et al., Am J Physiol Heart Circ Physiol, 2006, 291:3136-3143; Differential mechanisms mediating depressor and diuretic effects of anandamide Li and Wang, J Hypertens (2006) 24: 2271-6; Endocannabinoid Regulates Blood Pressure via Activation of the TRPV1 in Wistar Rats Fed a High Salt Diet, Wang et al., J Pharmacol Exp Ther. 2007 May; 321(2):763-9; Function and regulation of epithelial sodium transporters in the kidney of a salt-sensitive hypertensive rat model, Li and Wang, J Hypertens. 2007 May; 25(5):1065-72; TRPV1 Gene Knockout Impairs Postischemic Recovery in Isolated Perfused Heart in Mice (Wang et al., Circulation. 2005; 112:3617-3623); A Novel Mechanism Contributing to Development of Dahl Salt-Sensitive Hypertension (Wang et al., Hypertension, 2006, March; 47(3):609-14); Impaired vasodilation in response to perivascular nerve stimulation in mesenteric arteries of TRPV1-null mutant mice, Wang et al., Journal of Hypertension, 2006, 24:2399-2408; and Function and regulation of epithelial sodium transporters in the kidney of a salt-sensitive hypertensive rat model, Li, et al., Journal of Hypertension 2007, 25:000-000.

### Example I

### VR1-Mediated Depressor Effects During High-Salt Intake Role of Anandamide

Experiments were approved by the Institutional Animals Care and Use Committee. Experiments were performed using male Wistar rats (Charles River Laboratory, Wilmington, Mass.). Rats (6 weeks old) were housed in the animal facility for 1 week before the experiments. Rats were then randomly assigned to a normal-sodium (NS) diet (0.4% of Na+ by weight, Harlan Teklad) or HS (4% of Na+ by weight; Harlan Teklad) for 3 weeks. Rats drank water ad libitum throughout the experiment.

Surgical Preparation and MAP measurements: The rats were anesthetized with ketamine and xylazine (80 and 4 mg/kg intraperitoneally, respectively) for implantation of vascular catheters or with urethane (1.5 mg/kg intraperitoneally) throughout protocol 2. The left jugular vein and carotid artery were cannulated under anesthesia for administration of drugs or monitoring of mean arterial pressure (MAP) and heart rate (HR) with a Statham 231D pressure transducer coupled to a Gould 2400s recorder (Gould Instruments).

Experimental Protocols:

Protocol 1: Rats fed a NS or HS diet were randomly assigned to 5 groups for intravenous injection of vehicle or MethA alone or in combination with SR141716A (a CB1 receptor antagonist), CAPZ (a VR1 receptor antagonist), or the combination of the 2 (n=5 to 8). Baseline MAP and its response to the aforementioned drugs were determined 3 hours after surgery when animals were fully awake and unrestrained as described previously. MethA was administered in increasing doses (0.5, 5, and 15 mg/kg intravenous bolus) in 3 subgroups of rats (each group for each dose). To examine the effects of SR141716A and CAPZ on MethA-induced changes in MAP, MethA (5 mg/kg) was injected 20 minutes or 10 minutes after intravenous injection of SR141716A (3 mg/kg), CAPZ (3 mg/kg), or the combination of the 2. The doses and time frames for injection of these drugs were based on the results of our previous7 and current studies (see results) showing that SR141716A and CAPZ caused transient elevation in blood pressure in rats fed HS diet, which lasted for no more than 15 minutes and 7 minutes after injection of these drugs, respectively. Thus, MethA was injected when baseline MAP was restored and stable, which happened to be 20 minutes and 10 minutes after injection of SR141716A and CAPZ, respectively.

Protocol 2: To determine the role of VR1 as a depressor during HS intake, a selective VR1 receptor agonist, capsaicin (10 and 30 mg/kg bolus), or vehicle was intravenously injected into the anesthetized rats fed a NS or HS diet (n=5 to 6). Given that Capsaicin is an irritant and causes severe pain in conscious rats, this protocol was performed under in anesthetized rats. Each dose of injection was separated by a 30-minute interval. To determine the specificity of capsaicin, CAPZ (3 mg/kg) was administered 10 minutes before capsaicin injection in a separate group.

Protocol 3: Rats fed a NS or HS diet were euthanized by decapitation without subjecting to acute experiments. Mesenteric resistance arteries were collected for Western blot analysis of VR1 and CB1, and for immunohistochemical staining of VR1 and CGRP.

Compounds: Methanandamide (Sigma), capsaicin (Sigma), and SR141716A (provided by Dr Kaminski) were dissolved in ethanol (10% v/v), Tween-80 (10% v/v), and normal saline. Capsazepine (Calbiochem) was dissolved in dimethyl sulfoxide (10%, v/v), Tween-80 (10%, v/v), and normal saline.

Statistical Analysis: Values are expressed as mean +/−SE. Differences between 2 groups or before and after treatment were analyzed by using the unpaired or paired Student t test. The differences among groups were analyzed using 1-way ANOVA followed by a Bonferroni adjustment for multiple comparisons. Differences were considered statistically significant a†P<0.05.

### Example II

The following example demonstrated impaired vasodilation in response to perivascular nerve stimulation in mesenteric arteries of TRPV1-null (TRPV1 impaired) mutant mice.

TRPV1 gene knockout impairs postischemic recovery in isolated perfused heart in mice.

Male TRPV1 gene knockout (TRPV1−/−) strain B6.129S4-TRPV1tm1Jul and control wild-type (WT) strain C57BL/6J mice were used (Jackson Laboratory, Bar Harbor, Me.). Mice were heparinized (500 U/kg IP) and anesthetized with urethane (780 mg/kg IP). Hearts from TRPV1−/− and WT mice were cannulated and retrograde perfused at 37° C. and 80 mm Hg with Krebs-Henseleit buffer (118 mmol/L NaCl, 4.7 mmol/L KCl, 1.2 mmol/L MgSO4, 1.2 mmol/L KH2PO4, 2.5 mmol/L CaCl2, 25 mmol/L NaHCO3, 0.5 mmol/L Na-EDTA, and 11 mmol/L glucose, saturated with 95% O2/5% CO2, pH 7.4) through the aorta in a noncirculating Langendorff apparatus, as described previously. A water-filled balloon was inserted into the left ventricle and adjusted to a left ventricular end-diastolic pressure (LVEDP) of 5 to 8 mm Hg. The distal end of the catheter was connected to a Digi-Med Heart Performance Analyzer via a pressure transducer, and coronary flow (CF) was measured by a flowmeter with an online probe. Hearts were paced at 350 bpm except during ischemia, and pacing was reinitiated 2 minutes after reperfusion. After a 25-minute equilibration period, hearts were subjected to 40 minutes of no-flow normothermic global ischemia, followed by 30 minutes of reperfusion. The LVEDP, left ventricular developed pressure (LVDP) (peak systolic minus end-diastolic left ventricular pressure), and CF were measured during the process. The experiments were approved by the Michigan State University Animal Care and Use Committee.

Five experimental series were conducted, as follows.

Experiment 1: Perfusion With Capsazepine: These experiments were performed to determine the function of the TRPV1 receptor during ischemia/reperfusion injury and the difference of cardiac functions between long-term and short-term absence of the TRPV1 receptor. As normal controls (nonischemic), WT and TRPV1−/− hearts were perfused for 95 minutes. In experiments performed in the absence of the TRPV1 receptor antagonist, hearts from WT and TRPV1−/− mice were subjected to ischemia and reperfusion as described above, and cardiac function was measured. For acute blockade of the TRPV1 receptor, hearts from WT and TRPV1−/− mice were perfused with Krebs-Henseleit buffer for a 25-minute equilibration period, and capsazepine (10-6 mol/L), a selective antagonist of the TRPV1 receptor, was added to the perfusate 5 minutes before ischemia. Two additional concentrations of capsazepine, 10-5 and 10-7 mol/L, and another selective TRPV1 receptor antagonist, ruthenium red (10-6 mol/L), were used in WT hearts. WT hearts were also perfused with capsazepine (10-6 mol/L) for 95 minutes without ischemia/reperfusion injury as an additional control.

Experiment 2: Perfusion With CGRP: The effect of exogenous CGRP on cardiac function during ischemia and reperfusion was assessed. Hearts from WT and TRPV1−/− mice were subjected to a 25-minute equilibration period and perfused with CGRP (10-7 mol/L) added to the perfusate 5 minutes before ischemia. Additional WT hearts were perfused with CGRP8-37 (a selective CGRP antagonist; 10-6 mol/L) added to the perfusate 5 minutes before the addition of CGRP (10-7 mol/L) to confirm the specificity of the CGRP effect.

Experiment 3: Perfusion With CGRP₈₋₃₇: To determine the role of endogenous CGRP during ischemia and reperfusion in both WT and TRPV1−/− hearts, CGRP₈₋₃₇ was added to the perfusate alone. Hearts from WT and TRPV1−/− mice were perfused with CGRP₈₋₃₇ (10-6 mol/L) added to the perfusate 5 minutes before ischemia. Two additional concentrations of CGRP₈₋₃₇7, 10-5 mol/L and 10-7 mol/L, were also used in WT hearts. WT hearts were perfused with CGRP₈₋₃₇ (10-6 mol/L) for 95 minutes without ischemia/reperfusion injury as a control.

Experiment 4: Perfusion With SP: Hearts from WT and TRPV1−/− mice were subjected to a 25-minute equilibration period and perfused with SP (10-6 mol/L) added to the perfusate 5 minutes before ischemia. Additional WT hearts were perfused with RP67580 (a selective neurokinin-1 [NK1] receptor antagonist; 10-6 mol/L) added to the perfusate 5 minutes before the addition of SP (10-6 mol/L) to confirm the specificity of the SP effect.

Experiment 5: Perfusion With RP67580: Hearts from WT and TRPV1−/− mice were subjected to a 25-minute equilibration period and perfused with RP67580 (10-6 mol/L) added to the perfusate 5 minutes before ischemia. Two additional concentrations of RP67508, 10-5 mol/L and 10-7 mol/L, were used in WT hearts. WT hearts were perfused with RP67580 (10-6 mol/L) for 95 minutes without ischemia/reperfusion injury as a control.

Measurement of SP: The WT and TRPV1−/− hearts were cut into pieces and put into the tube containing 1.5 mL Krebs-Henseleit buffer that was saturated with 95% O₂/5% CO₂ continuously for 70 minutes (normal control group) as described. (Wang, et al., Circulation 112: 3617-23, 2005). In experimental groups, the WT and TRPV1−/− hearts were saturated with 95% O₂/5% C O₂ for 30 minutes after 40 minutes of incubation without O₂ (the ischemia/reperfusion group). Additional WT and TRPV1−/− hearts were treated the same as in the ischemia/reperfusion group except that capsazepine (10-6 mol/L) was added to the solution. The samples were purified and analyzed by radioimmunoassay (SP rat RIA kits; Peninsula Laboratories) as described previously for determination of SP release, which was normalized by the heart weight.

Statistical Analysis

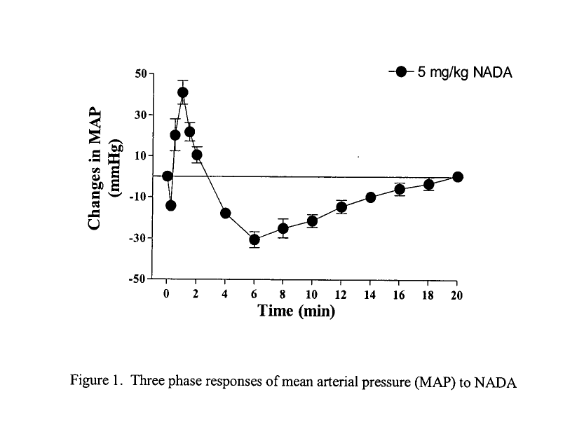

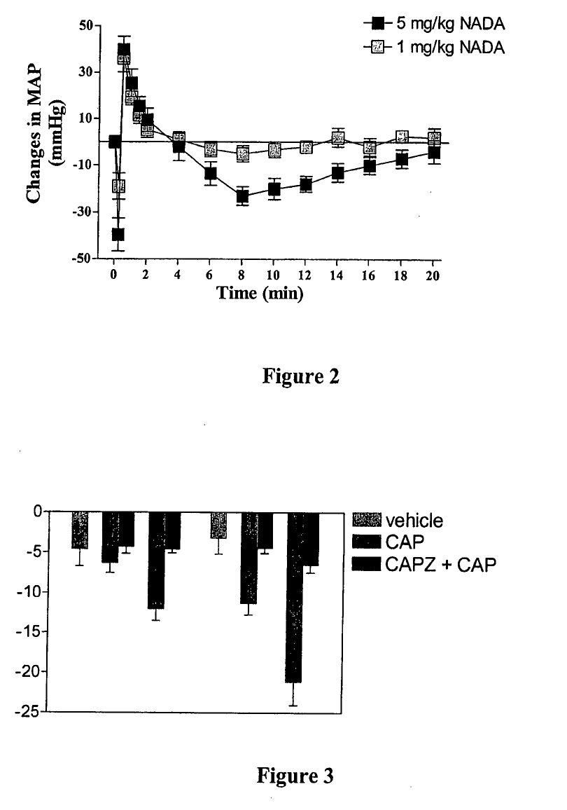

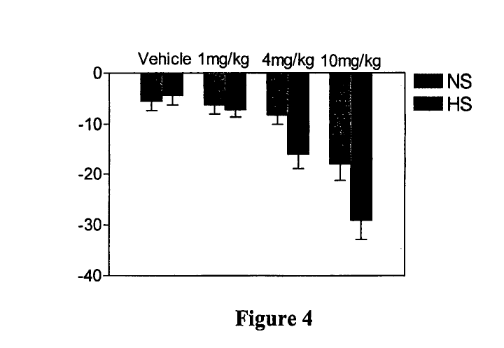

Values are expressed as mean ±SEM. Groups of WT hearts subjected to ischemia/reperfusion injury and TRPV1−/− hearts subjected to ischemia/reperfusion injury represent the same animals in FIGS. 1 to 5. Differences among groups with multiple measurements over time were determined by ANOVA (2-way ANOVA) for repeated measurements, and differences between means were identified by the least significant difference test. Comparisons among groups measured at the end of the ischemia/reperfusion experiments in the bar charts of each Figure and in SP release experiments were performed by 1-way ANOVA analysis followed by the Tukey-Kramer multiple comparison test. The difference in NK1 receptor expression between WT and TRPV1−/− hearts was determined by the Student t test. The results were considered statistically significant a†P<0.05.

### Example III

### Diuresis and Natriuresis Caused by Activation of VR1-Positive Sensory Nerves in Renal Pelvis of Rats

Male Wistar rats weighing 312.5 grams (Charles River Laboratories, Wilmington, Mass.) were housed in the animal facility 1 week before the experiment. Rats were divided into 7 groups and subjected to the following treatments: (1) control (vehicle), 5% ethanol, and 5% tween 80 in saline given via left renal pelvis perfusion (LRPP) (vehicle LRPP, n=5); (2) capsaicin (CAP), a selective VR1 receptor agonist, given at 2.4 nmol intravenously (CAP intravenous); (3) CAP at 2.4 nmol given via LRPP (CAP LRPP); (4) capsazepine (CAPZ), a selective VR1 receptor antagonist, given at 24 nmol via LRPP before CAP perfusion (CAPZ-CAP LRPP, n=6); (5) CAPZ given at 24 nmol via LRPP (CAPZ LRPP, n=5); (6) acute left renal denervation (RD) before CAP perfusion via LRPP (RD-CAP LRPP); and (7) acute left RD before vehicle LRPP (RD-vehicle LRPP, n=5). An additional 4 groups of rats (n=5 in each) were used for determining the effect of vehicle or CAP on renal excretory function at doses of 0.04, 0.4, and 2.4 nmol given via LRPP.

Rats were intraperitoneally administered pentobarbital sodium at 50 mg/kg and maintained with an intravenous infusion at 10 mg/kg per hour at 50 mL/min. Catheters were placed in the right jugular vein for administration of drugs and in the right carotid artery for monitoring mean arterial pressure (MAP) with a Statham 31D pressure transducer coupled to a Gould 2400s recorder (Gould Instrument Systems, Valley View, Ohio). Polyethylene (PE-50) catheters were inserted into both of the ureters via a midriff incision. A fine outlet tube of MD-2000 (ID 0.18/OD 0.22 mm; BASI, West Lafayette, Ind.) was placed inside the PE-50 catheter, with its tip in the renal pelvis during a 3-minute perfusion of drug at the rate of 20 mL/min that did not change renal pelvis pressure.

The experiments started 1.5 hours after the end of the surgery. LRPP consisted of 2 3-minute segments, i.e. CAPZ perfused within the first 3-minute segment, and CAP within the second 3-minute segment. In the case when CAP or CAPZ was perfused alone, the other segment was perfused with vehicle. In controls, vehicle was perfused in both segments without perfusion of CAP or CAPZ. Urine samples were collected for 10 minutes before and after each experiment protocol for analyses of urine flow rate (Uflow) and urinary sodium excretion (UNa). Urinary sodium excretion was measured using a flame photometer (model IL-943; Instrumentation Laboratory). At the end of experiment, blood samples were collected for determining plasma CGRP levels.

Verification of Acute Renal Denervation: The left kidney was denervated by transecting left renal nerves and painting the renal artery with 10% phenol in absolute ethanol. Thereafter, a bipolar stimulating electrode was placed on the left lumbar sympathetic chain above the left kidney. A flexible fiberoptic probe was placed into the renal cortex and connected to a laser Doppler flowmeter (Periflux System 5000; Perimed) for monitoring renal cortex blood flow (rCBF). A Grass S88 stimulator delivered conventional rectangular pulses of 0.2-ms duration, 15-V mplitude, and 4-Hz frequency for a total stimulation period of 1 minute. The absence (5% change) of a decrease in rCBF was taken as evidence of the completeness of left renal denervation. 16

Compounds: Capsaicin (Sigma) was dissolved in ethanol (5% v/v), tween 80 (5% v/v), and saline to make a stock solution of 65 nmol/μL, and was diluted in saline for intravenous and renal pelvis perfusion. Capsazepine (Calbiochem, San Diego, Calif.) was dissolved in DMSO (10% v/v), tween 80 (10% v/v), and saline to make a stock solution of 53 mmol/μL, and was diluted in saline for renal pelvis perfusion.

Statistical Analysis

All values were expressed as means SE. The differences among groups were analyzed using 1-way ANOVA followed by the Tukey-Kramer multiple comparison tests. The time course of Uflow after acute left renal denervation was analyzed using 2-way ANOVA with repeated measures for 1 factor. Comparisons of MAP before and after administration of drugs and rCBF before and after acute renal denervation were performed by the use of a paired t test. Differences were considered statistically significant a†P<0.05.

Results:

Transient receptor potential vanilloid type 1 (TRPV1) channels play a role in preventing high salt (HS) induced increases in blood pressure (BP), but mechanisms for TRPV1 activation by HS is unclear. The following example shows that an increased sensitivity of BP to N-arachidonoyl dopamine (NADA), an endovanilloid metabolized from dopamine, occurs during HS intake.

NADA (1, 4, 10 mg/kg, iv) dose-dependently decreased mean arterial pressure (MAP) in conscious Wistar male rats fed a HS diet for 10 days, and its depressor effect was greater in High salt (HS) vs. normal salt (NS)-treated rats (16±3 vs. 8±2 mmHg, p<0.05, n=6-7). FIGS. 1-2, and 4.

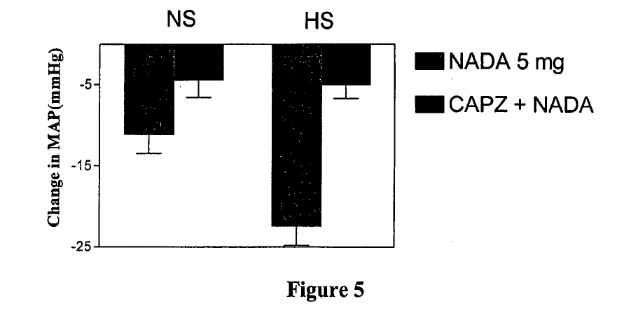

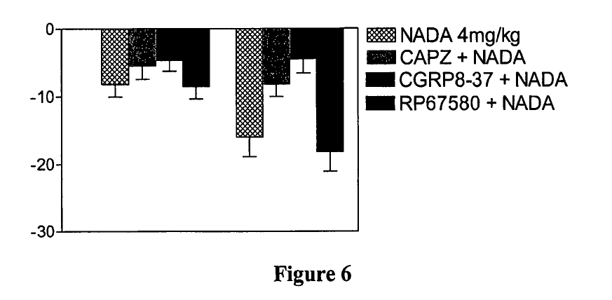

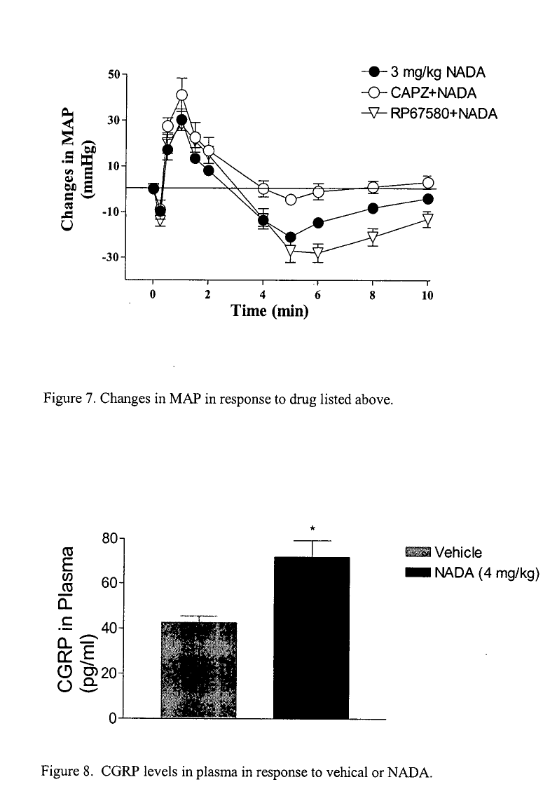

NADA (4 mg/kg) induced depressor effect was abolished by capsazepine (CAPZ, 3 mg/kg), a selective TRPV1 antagonist FIG. 3 and FIG. 7, or CGRP₈₋₃₇ (1 mg/kg/min), a selective CGRP receptor antagonist (p<0.05). FIG. 5 and FIG. 6.

Capsaicin (CAP, 10 or 30 μg/kg, iv), a selective TRPV1 receptor agonist, or CGRP (1 or 5 μg/kg, iv), dose-dependently decreased MAP in HS and NS rats, with a greater effect in the former (CAP, 11±2 vs. 6±1; 21±3 vs. 12 ±2; CGRP, 22±3 vs. 12±2; 41±4 vs. 25 ±3, p<0.05).

CGRP levels in plasma were higher in HS compared to NS rats (58.7±5.7 vs. 40.3±4.6 pg/ml, p<0.05), and that NADA (4 mg/kg) caused a greater increase in plasma CGRP levels in HS compared to NS rats (84.2±7.2 vs. 55.3±4.6 pg/ml, p<0.05). FIG. 8.

TRPV1 receptor protein expression in the mesenteric arteries was increased in HS compared to NS-treated rats (0.81±0.06 vs. 0.59±0.04, p<0.05).

The data show that 1) HS upregulates mesenteric TRPV1 expression; 2) HS increases sensitivity of BP to NADA; 3) HS increases basal and NADA-induced release of CGRP; and 4) the enhanced depressor effect induced by NADA during HS intake is blocked by antagonists of TRPV1 or CGRP receptors.

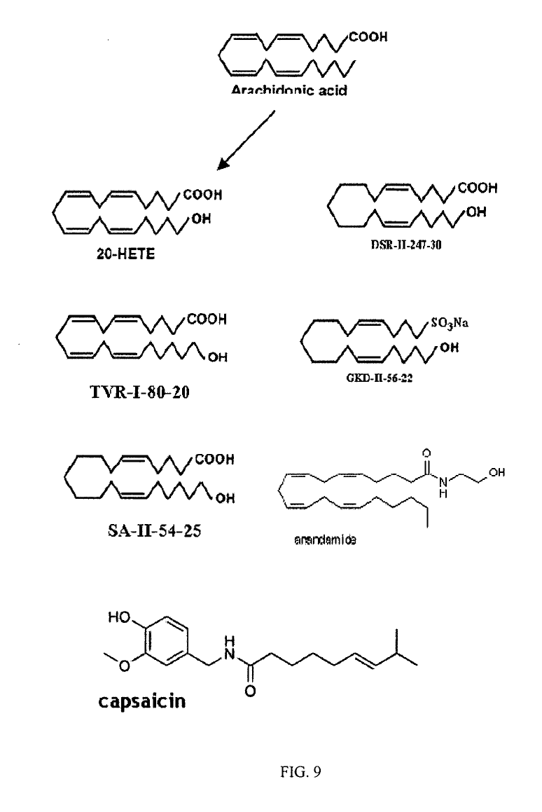

These results indicate that NADA, in addition to other TRPV1 agonists (FIG. 9) may serve as a novel endogenous TRPV1 agonist to prevent salt induced increases in BP via enhancing CGRP release.

### Example IV

### TRPV1-Mediated Depressor Effect: Role of Cytochrome P450 Metabolite 20-HETE

20-hydroxyeicosatetraenoic acid (20-HETE), a monooxygenation product of arachidonic acid (AA) metabolized by cytochrome P450 (CYP) enzyme, omega/omega-1-hydroxylase, has been shown to play a role in cardiovascular regulation. The effects of 20-HETE are cell- and tissue-specific given the diverse function of this CYP-AA metabolite.

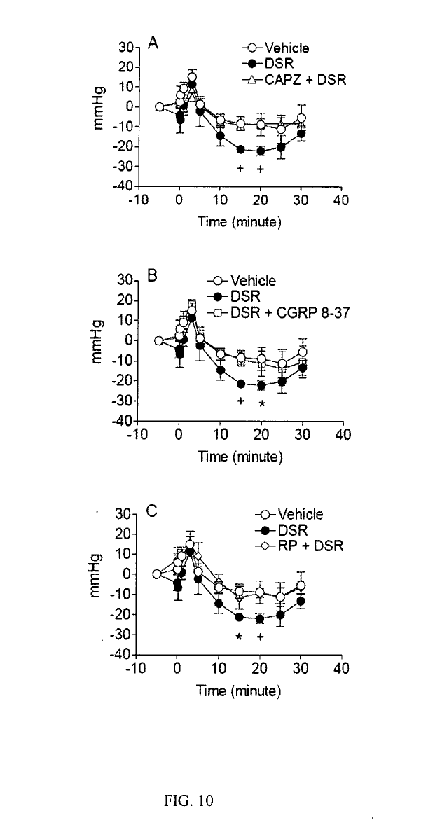

Several analogs of 20-HETE, which were equally potent as the parent 20-HETE in vitro assays, were administered IV via right jugular veins to male Wistar rats. Among the analogs tested in vivo, an analog named DSR-II-247-30 (DSR) was most potent in terms of its depressor effect. Mean arterial pressure (MAP) was significantly decreased by DSR at 1 mg/kg compared to vehicles (22.1±2.3 mmHg vs. 8.9±5.7 mmHg, n=5-6; P<0.05, respectively). FIG. 10A.

Capsazepine (CAPZ, 3 mg/kg), a selective TRPV1 antagonist, RP67580 (RP) (4 mg/kg), a selective neurokinin-1 receptor (NK1) antagonist, or CGRP 8-37 (3 mg/kg), a selective calcitonin-gene related peptide (CGRP) receptor antagonist, blocked DSR induced-depressor effects (8.5±2.5 mmHg, 9.4±2.3 mmHg, and 9.3±3.3 mmHg vs. 22.1±2.3 mmHg in DSR alone, P<0.05, respectively). FIGS. 10B and C.

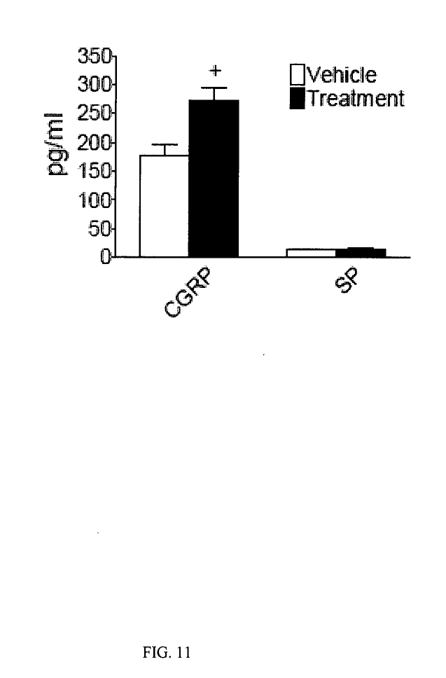

Radioimmunoassay showed that DSR significantly increased plasma CGRP levels (177.2±20.9 to 273.4±23.6 pg/mL, P<0.05) without changing plasma SP levels (13.5±0.2 to 14.5±1.6 pg/mL, P>0.05). FIG. 11.

Thus, activation of TRPV1 contributes to the depressor effect of 20-HETE, possibly via TRPV1-mediated release of CGRP and other sensory neuropeptides. These results indicate that 20-HETE may serve as an endogenous TRPV1 agonist to modulate blood pressure under pathophysiological conditions. Further, an analog of 20-HETE, 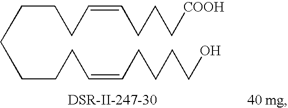
plays a depressor role via activation of the transient receptor potential vanilloid type 1 (TRPV1) channels expressed in sensory nerves.

Increased Depressor Response to N-Arachidonoyl-dopamine During High Salt Intake: Role of the TRPV1 Receptor

### Example V

N-oleoyl-dopamine (OLDA; Chu et al., 2003, The Journal of Biological Chemistry, Vol. 278, No. 16, Issue of April 18, pp. 13633-13639), was discovered by the inventors to protect cardiac tissue and activated TRPV1 channels.

Specifically, N-oleoyl-dopamine (OLDA) refers to an endovanilloid bioactive lipid originally found in the mammalian brain. OLDA selectively activated the transient receptor potential vanilloid type 1 (TRPV1) channel in vitro with a potency at least 30 times higher than capsaicin, a selective exogenous TRPV1 agonist. Thus the inventors further tested for and found that OLDA protected the heart against ischemia and reperfusion (I/R) injury via activation of the TRPV1 in wild type (WT) but not in gene-targeted TRPV1-null mutant (TRPV1^{-/-}) mice.

In particular, hearts of WT or TRPV1^{-/-} mice were Langendorffly perfused with OLDA (1.5×10⁻⁷M) in the presence or absence of capsazepine (CAPZ, 1×10⁻⁶ M), a selective TRPV1 antagonist; CGRP₈₋₃₇ (1×10⁻⁶ M), a selective calcitonin gene-related peptide (CGRP) receptor antagonist; or RP67580 (1×10⁻⁶ M), a selective neurokinin-1 (NK1) receptor antagonist, followed by 35 minutes of global ischemia and 40 minutes of reperfusion. The following measurements were made on the two types of heart tissue: left ventricular end-diastolic pressure (LVEDP), left ventricular developed pressure (LVDP), coronary flow (CF), and left ventricular (LV) peak positive rate of pressure change/rate of time change (+dP/dt) were evaluated after I/R.

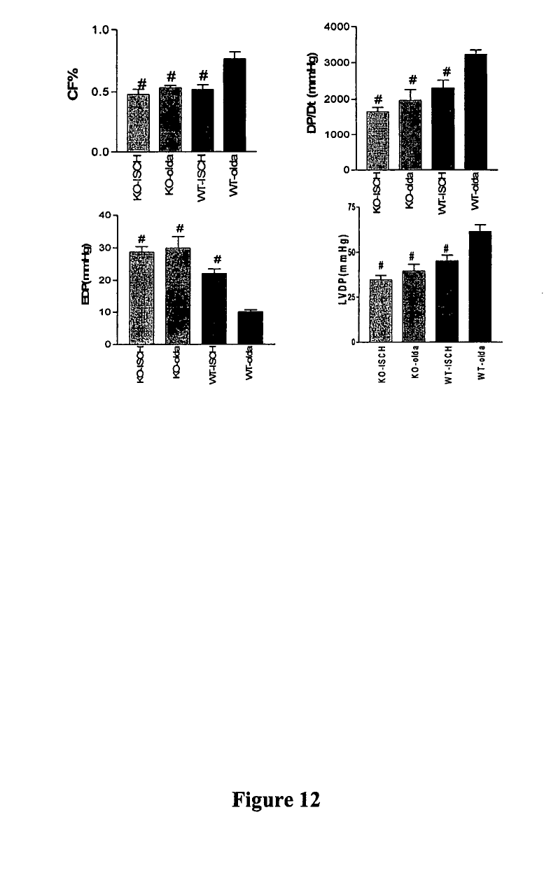

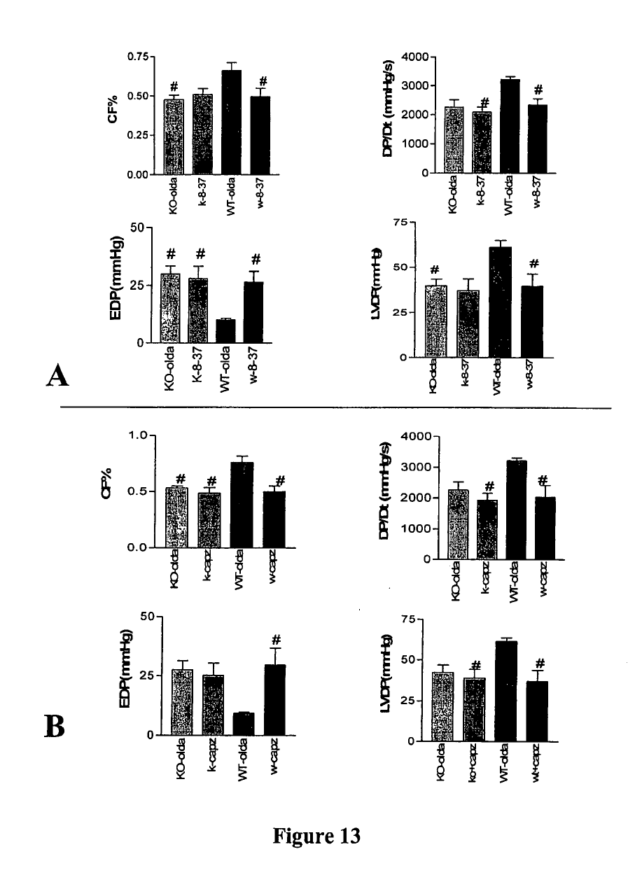

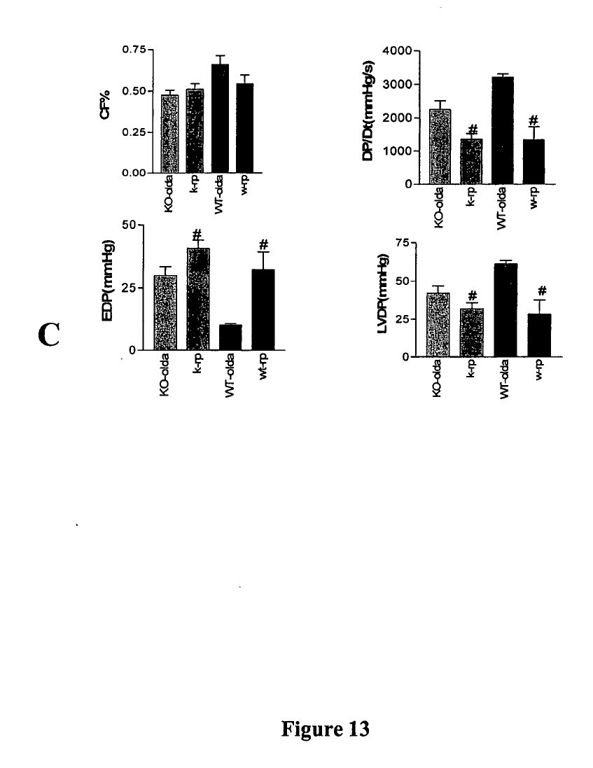

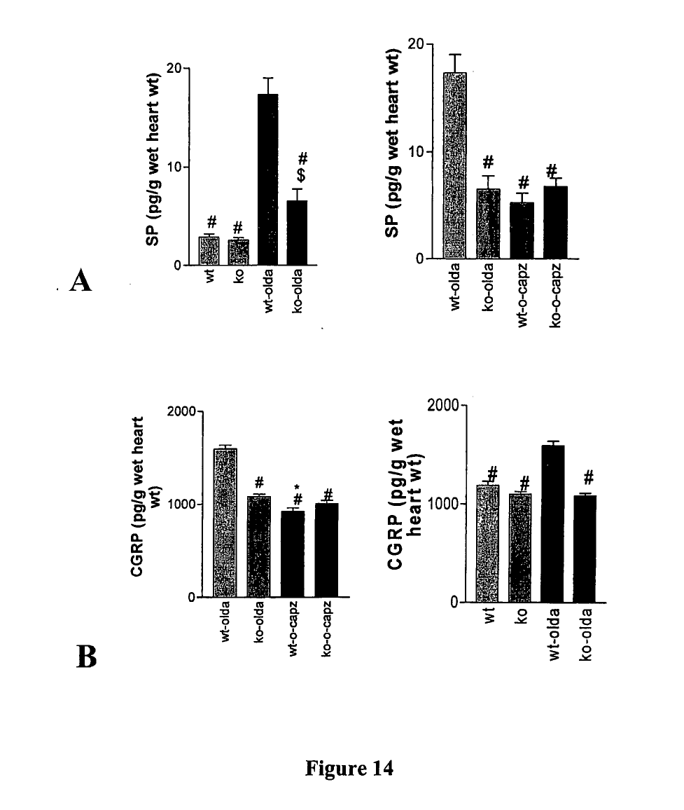

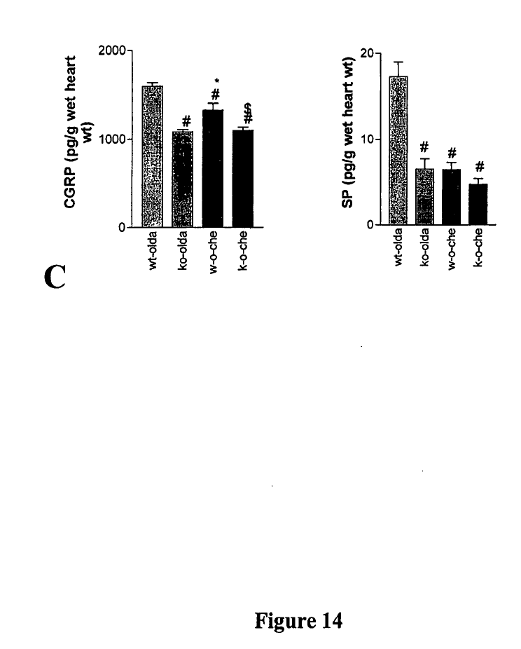

Thus, OLDA, via activating TRPV1 leading to CGRP and SP release, exerts a cardiac protective effect during I/R injury in WT hearts. FIG. 12 shows exemplary N-oleoyl-dopamine (OLDA) improved recovery of cardiac function after ischemia and reperfusion (I/R; ISCH) in WT (wild-type; w) but not TRPV1^{-/-} (knock-out; KO; k) hearts by increasing left ventricular developed pressure (LVDP) (WT: 45.2±3.2 vs 61.3±3.8 mmHg; p<0.05; TRPV-1^{-/-}34.7±2.5 vs 39.8±3.7 mmHg; p>0.05), coronary flow (CF) (WT: 52±4 vs 76±6%, p<0.05; TRPV^{-/}−, 48±4 vs 53±2%, p>0.05), and left ventricular (LV) peak positive dP/dt (+dP/dt) (WT: 2303±214 vs 3227±126 mmHg/s, p<0.05; TRPV^{-/-}1656±119 vs 1967±299 mmHg/s, p>0.05), and by decreasing left ventricular end-diastolic pressure (LVEDP; EPD) (WT: 22.1±1.4 vs 10.1±0.6 mmHg, p<0.05; TRPV^{-/-}28.7±1.7 vs 29.9±3.5 mmHg, #=p>0.05). (with OLDA vs without OLDA, respectively). FIG. 13 shows exemplary protective effect of N-oleoyl-dopamine (OLDA) in WT (wild-type; w) hearts was abolished by A) Calcitonin-Gene Related Peptide (CGRP₈₋₃₇; K-8-37), B) capsazepine (CAPZ), and C) RP67580 (rp). FIG. 14 shows an exemplary radioimmunoassay where N-oleoyl-dopamine (OLDA) induced significantly higher A) Calcitonin-Gene Related Peptide (CGRP) and Substance P(SP) release in WT (wild-type; w) compared to TRPV1^{-/-} hearts (p<0.05), B) CAPZ blocked increased CGRP and SP release in WT hearts and C) a Protein Kinase C(PKC) inhibitor, chelerythrine (che) blocked increased CGRP and SP release in WT hearts. The protective effect of OLDA is void in TRPV1^{-/-} hearts, supporting the notion that TRPV1 mediates OLDA induced protection against cardiac I/R injury.

### Example VI

This example demonstrates that N-arachidonoyl-dopamine (NADA), an endovanilloid, shows depressor activity via activation of the transient receptor potential vanilloid type 1 (TRPV1) channels during high salt intake.

In brief: Wistar rats were fed a normal (NS, 0.4%) or high (HS, 4%) sodium diet for 10 days, then artery and vein were cannulated for measurement of mean arterial pressure (MAP) or for injection of drugs and collection of plasma. Radioimmunoassay and Western blot were used to determine the plasma CGRP level and TRPV1 protein content, respectively.

Results: NADA-induced dose-dependent decrease in MAP was greater in HS-compared to NS-treated rats, and was abolished by capsazepine (CAPZ), a selective TRPV1 antagonist, or CGRP₈₋₃₇, a selective calcitonin gene-related peptide (CGRP) receptor antagonist, but not by SR141716A, a selective cannabinoid receptor 1 (CB1) antagonist. Capsaicin, a selective TRPV1 receptor agonist, or CGRP dose-dependently decreased MAP in NS- or HS-treated rats with a greater effect in the latter. Baseline and NADA-induced increases in plasma CGRP levels were higher in HS- compared to NS treated rats. TRPV1 protein expression in mesenteric arteries was higher in HS compared to NS-treated rats. In vitro, NADA caused a greater CGRP release from mesenteric arteries of HS-compared to NS-treated rats, which was blocked by CAPZ.

Methods:

Animal Preparation: Male Wistar rats (7 weeks old, Charles River Laboratory, Wilmington, Mass.) were housed in the animal facility for 1 week before the experiment. Rats were grouped randomly and pair-fed a NS (0.4% of Na⁺ by weight) or HS (4% of Na⁺ by weight) diet for 10 days. The rat chow was purchased from Harlan Teklad Diets (Madison, Wis.). Drinking water was available ad libitum throughout the experiment. All protocols were in accordance with the National Institutes of Health Guidelines for the Care and Use of Laboratory Animals.

Surgical Preparation Rats were anesthetized with ketamine and xylazine (80 and 4 mg/kg intraperitoneally, respectively) for cannulation or with urethane (1.5 g/kg intraperitoneally) for capsaicin administration. The left jugular vein, carotid and femoral artery were cannulated under anesthesia for injection of drugs or monitoring of mean arterial pressure (MAP) and heart rate (HR).

Experimental Protocols:

Protocol 1: Rats fed a NS or HS diet were divided into 5 groups for intravenous administration of vehicle or NADA alone or in combination with CAPZ (a TRPV1 receptor antagonist), SR141716A (a CB1 receptor antagonist), or CGRP₈₋₃₇ (a CGRP receptor antagonist). Three hours after surgery, baseline MAP and its response to the above-mentioned drugs were obtained with the rats fully awake and unrestrained [Wang et al., Am J Physiol 2004; 287:H1868-H[874]. NADA was injected in increasing doses (1, 4, and 10 mg/kg intravenous bolus) in 3 subgroups of rats (each group for each dose). To determine the role of TRPV1 and CB 1 receptor in NADA-induced hypotension, NADA (4 mg/kg) was injected 10 minutes or 20 minutes after intravenous injection of CAPZ (3 mg/kg) or SR141716A (3 mg/kg), respectively. These doses of CAPZ and SR141716A have been shown to be effective in inhibiting the anandamide-induced hypotension in vivo [Wang et al., Hypertension 2005; 46:986-991; Lake et al., Hypertension 1997; 29:1204-1210; Akerman et al., Br J Pharmacol 2003; 140:718-724].

To examine the effect of CGRP₈₋₃₇ on NADA-induced hypotension, CGRP₈₋₃₇ was intravenously administered at the rate of 1 mg/kg/min for 2 minutes followed by injection at the rate of 0.5 mg/kg/min for 15 minutes. NADA (4 mg/kg) was injected 1 minute after CGRPs-37 administration. The effectiveness of the dose and time frame for CGRP₈₋₃₇ injection was verified by blockade of the hypotensive response to capsaicin (30 μg/kg) (n=5).

Protocol 2: To examine the role of TRPV1 or CGRP receptor as a depressor during HS intake, MAP responses to administration of capsaicin (10 and 30 μg/kg), a selective TRPV1 receptor agonist, or CGRP (0.5, 1, and 5 μg/kg) were determined in anesthetized or conscious rats fed a NS or HS diet. Each dose of injection was separated by a 30-minute interval. To determine the specificity of capsaicin, CAPZ (3 mg/kg) was administered 10 minutes before capsaicin injection in a separate group.

Protocol 3: To determine the effect of NADA on CGRP release from perivascular sensory nerves innervating mesenteric arteries in vitro, mesenteric arteries were collected from rats fed a NS or HS diet, and equilibrated for 1 h in a Krebs' solution (5% CO₂ and 95% O₂; 37oC) as described previously [5,22]. After an 1 h stabilization period, the arteries were transferred to another chamber containing 1 ml of Krebs's solution with the addition of 0.05% BSA and drugs including NADA (1 or 10 μM) or CAPZ (10 μM). The tissues were removed after 20 min incubation, and the solutions were collected and evaporated. The pellets were stored at −80oC for CGRP immunoassay. At the end of the experiment, the tissues were blotted and weighted. The highest concentration of NADA (10 μM) and CAPZ (10 μM) did not show any cross-reactivity with CGRP. The results are expressed as pg of milligram of tissues.

Protocol 4: To determine plasma CGRP levels at baseline and in response to NADA, vehicle or NADA (4 mg/kg) was injected into conscious rats fed a NS or HS diet. Eight minutes after injection, when the depressor phase approached to the lowest point, the rats were sacrificed for collection of blood for plasma CGRP assay.

Protocol 5: To determine TRPV1 protein expression in mesenteric arteries, rats fed a NS or HS diet were euthanized by decapitation without subjecting to acute experiments, mesenteric arteries were collected for Western blot analysis of TRPV1.

Radioimmunoassay: A rabbit anti-rat CGRP radioimmunoassay kit (Phoenix Pharmaceuticals) was used to determine CGRP content in plasma and tissue incubating buffer in vitro [Wang et al., Am JPhysiol 2004; 287:H1868-H[874]. This antibody has 100% cross-reactivity with rat a-CGRP and 79% with β-CGRP. There is no cross-reactivity with rat amylin, calcitonin, somatostatin, or substance P. The assay was performed following the manufacturer's protocol.

Western blot analysis: Western blot analysis was performed as previously described [6], with the use of primary antibody directed to TRPV1 (1:800, Santa Cruz Biotechnology), secondary antibody conjugated with horseradish peroxidase. The immuno-complexes on the membranes were detected using enhanced chemiluminescence ECL kit (Amersham Pharmacia Biotech) and exposed to film (Hyperfilm-ECL; Amersham Pharmacia Biotech). The films were scanned and analyzed by using the Image Quantity Program (Scion) to obtain integrated densitometric values. β-actin was used to normalize protein loaded on membranes.

Drugs: NADA (Cayman Chemical), capsaicin (Sigma), and SR141716A (kindly provided by Dr. Norbert E. Kaminski, Michigan State University) were dissolved in ethanol (10%, v/v), Tween-80 (10%, v/v), and normal saline. CAPZ (Calbiochem) was dissolved in dimethyl sulfoxide (10%, v/v), Tween-80 (10%, v/v), and normal saline. CGRP₈₋₃₇ (American Peptide) was dissolved in normal saline.

Statistical Analysis All values are expressed as mean ±SE. Differences between 2 groups or before and after treatment were analyzed by using the unpaired or paired Student's t test. The differences among groups were analyzed using one-way ANOVA followed by a Bonferroni's adjustment for multiple comparisons. Comparisons between groups at each experiment time point were determined by the use of two-way ANOVA followed by a Bonferroni's test. Differences were considered statistically significant at P<0.05.

Results:

Body weight was not significantly different between 2 groups after 10-day dietary treatment (NS, 275±6 g versus HS, 268±8 g, P>0.05). HS intake did not affect blood pressure considering that baseline MAP was not different between NS and HS-treated rats (109±5 mmHg versus 114±6 mmHg, P>0.05).

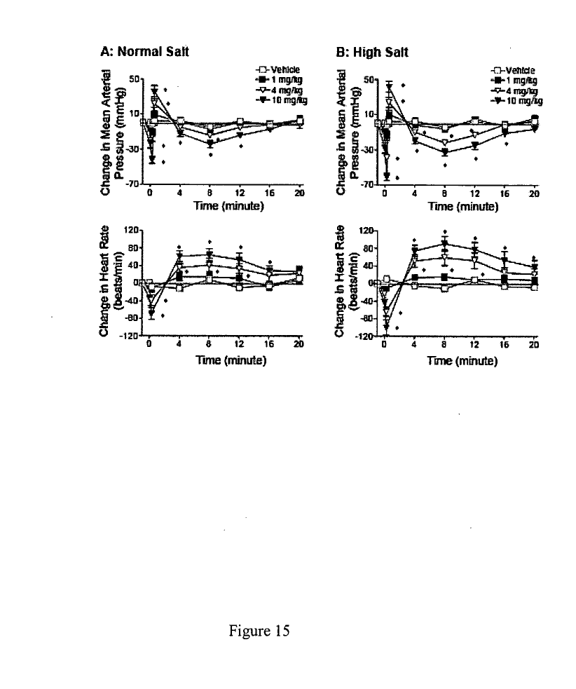

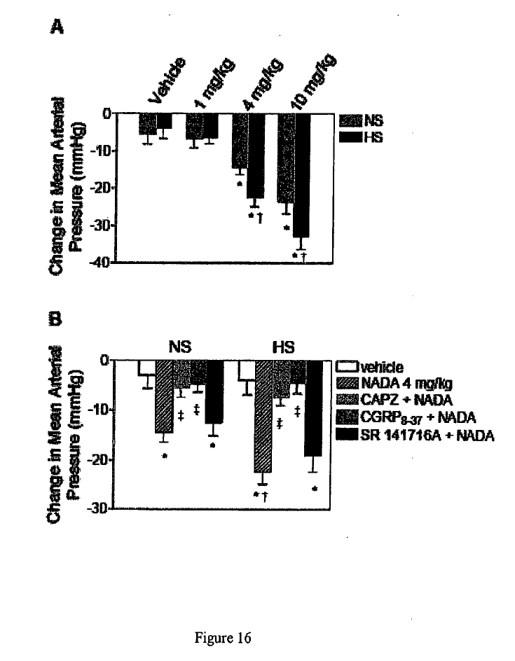

MAP and HR responses to intravenous injection of NADA in NS and HS-treated rats are shown in FIG. 15 and FIG. 16. Administration of NADA led to a triphasic MAP response in rats fed a NS or HS diet. The initial transient hypotension was followed by a brief pressor and a prolonged depressor phase. The prolonged depressor activity of NADA lasted for approximately 20 minutes, and reached the peak 7 to 9 minutes after administration of NADA. The depressor effect was associated with a pronounced increase in HR. NADA (1, 4, 10 mg/kg) induced peak decreases in MAP were dose dependent at 6±2 mmHg, 22±3 mmHg and 33±5 mmHg in HS-treated rats, respectively, which were significantly greater than that observed in NS-treated rats (7±3 mmHg, 14±2 mmHg, 24±4 mmHg, respectively). Blockade of the TRPV1 or CGRP receptors with CAPZ or CGRP₈₋₃₇ blocked the prolonged depressor effect but not the initial two phases of NADA at the dose of 4 mg/kg in both NS (6±2 mmHg, or 5±2 mmHg versus 14±2 mmHg, P<0.05) and HS-treated rats (7±2 mmHg, or 6±3 mmHg versus 22±3 mmHg, P<0.05). In contrast, SR141716A (3 mg/kg), a selective CB1 receptor antagonist, did not affect the prolonged depressor effect of NADA in NS and HS-treated rats (NS, 12±3 mmHg versus 14±2 mmHg; HS, 19±4 mmHg versus 22±3 mmHg, P>0.05). In addition, SR141716A did not affect the initial transient hypotensive and the brief pressor phases.

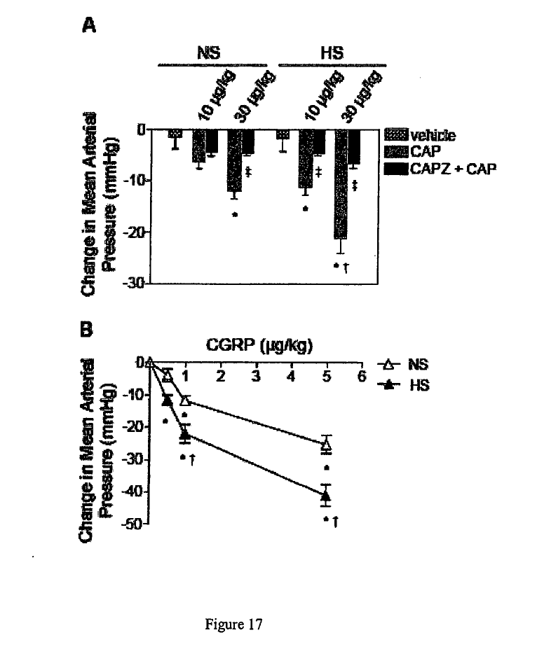

To examine the effects of TRPV1 activation by its specific agonist capsaicin on MAP in NS- and HS-treated rats, MAP responses to intravenous injection of capsaicin were studied (FIG. 17A). Activation of the TRPV1 by capsaicin caused a triphasic MAP response similar to that induced by NADA. Capsaicin produced a dose-dependent depressor effect in rats fed a NS or HS diet, with a greater effect in HS- than NS-treated rats (11±2 mmHg and 21±3 mmHg in HS-treated rats versus 6±1 mmHg and 12±2 mmHg in NS-treated rats, P<0.05). Moreover, CAPZ, a selective TRPV1 antagonist, blocked the decrease in MAP in NS-(4±1 mmHg; 5±1 mmHg) and HS-treated (4±1 mmHg and 6±2 mmHg) rats, considering that these values of MAP were not significantly different from those generated in vehicle-treated rats fed a NS (5±2 mmHg) or HS (3±2 mmHg) rats.

To determine the effect of HS intake on CGRP receptor function, MAP responses to intravenous administration of CGRP were examined in conscious rats fed a NS or HS rats as shown in FIG. 17B. CGRP (0.5, 1 and 5 μg/kg) caused dose-dependent decreases in MAP in rats fed a NS or HS diet. The magnitude of CGRP-induced decreases in MAP in HS-treated rats was significantly bigger than that in NS-treated rats (11±2, 22±3, and 41±3 mmHg in HS-treated rats versus 4±2, 12±2, and 25±3 mmHg in NS treated rats, P<0.05), respectively.

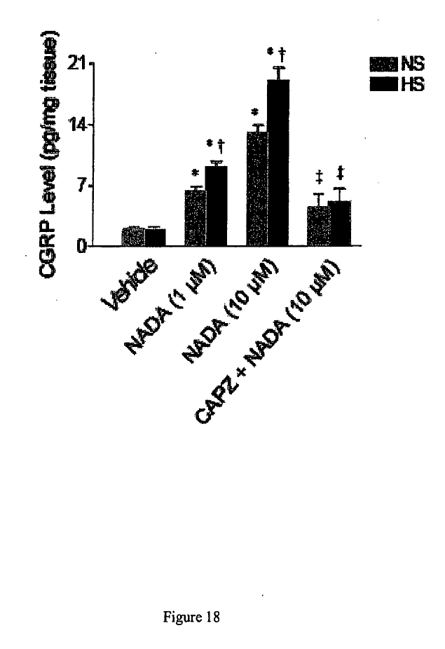

The effects of NADA on CGRP release in mesenteric arteries were studied in vitro as shown FIG. 18. NADA caused a dose-dependent CGRP release in mesenteric arteries isolated from rats fed a NS or HS diet. The CGRP release induced by NADA (1 and 10 μM) was significantly greater in HS-treated rats compared to NS-treated rats (9.16±0.68 and 19.00±1.56 pg/mg tissue in HS-treated rats versus 6.43±0.58 and 13.07±0.91 pg/mg tissue in NS-treated rats, P<0.05), respectively. Moreover, NADA (10 μM)-induced CGRP release was blocked by pretreatment with CAPZ (10 μM) in NS or HS treated rats.

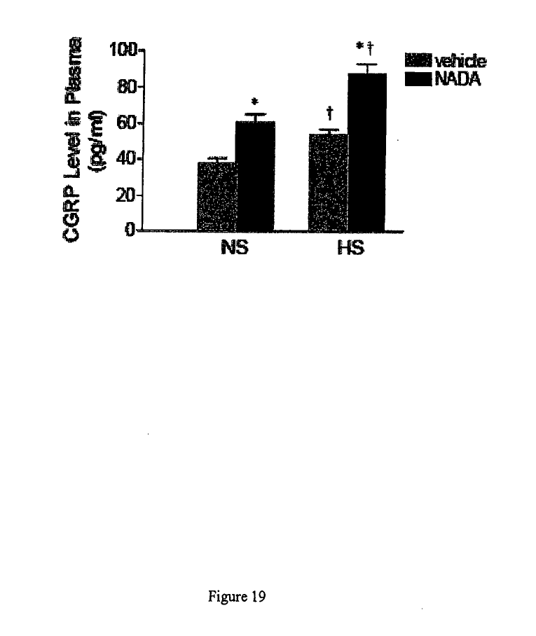

To examine the effects of NADA on CGRP release in vivo, plasma CGRP levels at baseline and in response to NADA were determined by radioimmunoassay as shown in FIG. 19. HS intake for 10 days significantly increased CGRP levels in plasma (NS, 38.0±2.9 pg/ml; HS, 53.8±3.3 pg/ml, P<0.05). Moreover, NADA at 4 mg/kg significantly increased plasma CGRP levels in both NS and HS-treated rats (NS, 38.0±2.9 pg/ml versus 60.6±5.1 pg/ml; HS, 53.8±3.3 pg/ml versus 87.7±5.6 pg/ml, P<0.05), with significantly higher magnitude in HS- compared to NS-treated rats (P<0.05).

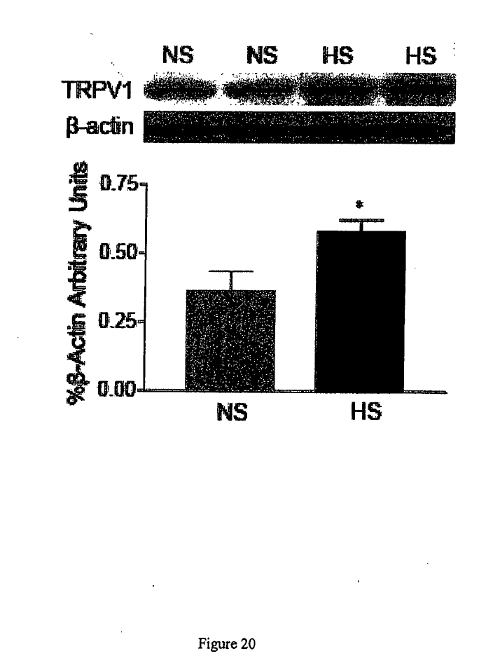

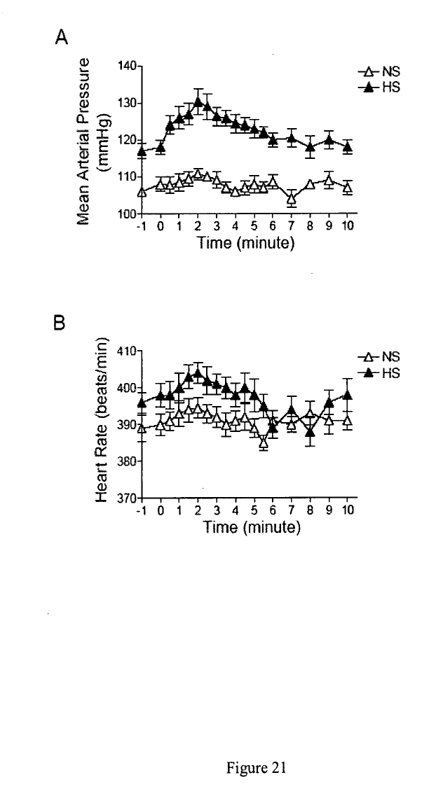

To determine the effect of HS intake on expression of TRPV1 in the perivascular sensory nerves innervating mesenteric arteries, Western blot analysis was performed and the results are shown in FIG. 20. HS intake for 10 days increased TRPV1 protein expression in mesenteric arteries when compared to rats fed a NS diet (0.58±0.04% of β-actin arbitrary versus 0.36±0.07% of β-actin arbitrary, P<0.05).

### Example VII

### Endocannabinoid Regulates Blood Pressure via Activation of the TRPV1 in Wistar Rats Fed a High Salt Diet

The inventors designed this example in order to examine the role of the endocannabinoids in blood pressure regulation during high salt (HS) intake.

HS (4% of Na⁺ by weight) intake for 3 weeks increased baseline mean arterial pressure (MAP, mmHg) when comparing to normal salt (NS, 0.4% of Na⁺ by weight)-treated male Wistar rats. Capsazepine (CAPZ, 3 mg/kg), a selective transient receptor potential vanilloid type 1 (TRPV1) antagonist, caused a greater increase in MAP (mmHg) in HS-treated compared to NS-treated rats (13±3 vs. 4±2, p<0.05) whereas calcitonin gene-related peptide (CGRP) dose-dependently decreased MAP in both HS- and NS-treated rats with a more profound effect in the former. HS increased plasma anandamide levels analyzed by Liquid Chromatography/Electrospray Tandem Mass Spectrometry (NS, 2.40±0.31 vs HS, 4.05+0.47 μmol/ml, p<0.05) and plasma CGRP levels determined by radioimmunoassay (NS, 36.6±3.8 vs HS, 55.7±6.4 pg/ml, p<0.05). MethA, a metabolically stable analog of anandamide, caused a greater CGRP release in mesenteric arteries isolated from HS-treated comparing to NS treated rats. Western blot showed that expression of receptor activity-modifying protein 1 (RAMP1), a subunit of the CGRP receptor, in mesenteric arteries was greater in HS-treated compared to NS-treated rats. These results show that HS intake increases production of anandamide, which may serve as an endovanilloid to activate TRPV1 leading to release of CGRP to blunt salt-induced increases in blood pressure. These data support the notion that TRPV1 may act as a molecular target for salt-induced elevation of endovanilloid compounds to regulate blood pressure.

Methods

Animal Preparation: Experiments were approved by the institutional Animals Care and Use Committee. Male Wistar rats (6 weeks old, Charles River Laboratory, Wilmington, Mass.) were housed in a temperature-controlled room with a 12:12-hour light/dark cycle. Rats were selected randomly to receive either a normal-sodium diet (NS, 0.4% of Na⁺ by weight) or a HS diet (HS, 4% of Na⁺ by weight) for 3 weeks. The diets were purchased from Harlan Teklad. All rats drank water ad libitum throughout the experiment.

Surgical Preparation The rats were anesthetized with ketamine and xylazine (80 and 4 mg/kg ip, respectively) for implantation of artery and vein catheters. The left jugular vein and carotid artery were cannulated for administration of drugs or for monitoring of mean arterial pressure (MAP) and heart rate (HR) with a Statham 231D pressure transducer coupled to a Gould 2400s recorder (Gould Instruments, Dayton, Ohio, USA). For conscious animal experiments, catheters were routed subcutaneously and exteriorized between the scapulae. Rats were quietly brought to home cages 3 hours after surgery, and MAP and HR were continuously recorded. Regular room light was turned on during experiments. The rats were allowed to acclimate to their surroundings for 15-30 minutes in their cages, i.e., until which time they ceased exploring the new environment and their blood pressures stabilized. Baseline MAP and HR were recorded for 15 minutes before administration of drugs (Supowit et al., 1997; Wang and Wang, 2004).

Protocol 1: To determine whether the TRPV1 is tonically activated during HS intake, rats fed a NS or HS diet were randomly assigned to 2 groups for intravenous injection of CAPZ (3 mg/kg), a selective TRPV1 antagonist, in conscious states.

Protocol 2: To determine the role of the CGRP receptor in rats fed a HS diet, MAP responses to injection of CGRP (1 and 5 pg/kg bolus) were measured in conscious rats fed a NS or HS diet. Thirty minutes were allowed for stabilization of blood pressure between injections.

Protocol 3: To determine plasma CGRP concentration in response to MethA (a metabolically stable analog of anandamide), vehicle or MethA (5 mg/kg) was administered into conscious rats fed a NS or HS diet. Fifteen minutes after injection, when the prolonged depressor phase approached to the lowest point, the rats were decapitated for collection of blood for plasma CGRP assay.

Protocol 4: To determine biochemical parameters in plasma and tissues, rats fed a NS or HS diet were sacrificed by decapitation without subjecting to acute experiments. Plasma and mesenteric arteries were collected for analysis of endocannabinoid and Western blot analysis of CGRP receptor components.

Protocol 5: To determine the effect of MethA (0.1 and 10 μM) on CGRP release from mesenteric arteries in vitro, mesenteric arteries from rats fed a NS or HS diet were collected and placed into ice-cold PBS solution. The arteries were dissected free of fat and connective tissues.

The preparations were incubated in a Krebs' solution of the following compositions (in mM): NaCl 119, NaHCO₃ 25, KH₂PO₄ 1.2, MgSO₄ 1.5, CaCl₂ 2.5, KCl 4.7, and D-glucose 11. The Krebs' solution was maintained at 37oC and gassed with 95% O₂ and 5% CO₂. After 1 h stabilization period, the arteries were transferred to another chamber containing 1 ml of Krebs' solution with the addition of 0.05% BSA and drugs. In addition, the tissue samples were incubated with CAPZ (10 μM), a selective TRPV1 antagonist, to determine the role of TRPV1 in MethA-induced CGRP release. The tissues were removed after 20 min incubation, and the solutions were collected. At the end of the experiment, the tissues were blotted and weighted. The solution in the tubes was evaporated and the pellets were stored at −80oC for CGRP immunoassay. The highest concentration of MethA (10 μM) and CAPZ (10 μM) did not show any cross-reactivity with CGRP. The results are expressed as pg of CGRP per milligram of tissue per 20 min.

Analysis of Endocannabinoid: Plasma was collected and stored at −80oC until lipid extraction. Lipid extracts were isolated from plasma spiked with D4-labeled anandamide as internal standards. The samples were analyzed by Liquid Chromatography/Electrospray Tandem Mass Spectrometry using electrospray positive program designed specific for anandamide as described previously (kindly provided by Drs. Joseph Leykam and Lijun Chen) (Bátkai et al., 2004; Giuffrida et al., 2000).

An electrospray-mass spectrometer (LCQ Deca, Thermo Electron Corporation) equipped with a Nova-PakC18 column (300×3.9 mm, 4 μm, Waters) was used for analysis. Anandamide standards eluted from the column after approximately 25 minutes. Diagnostic ions (protonated molecular ions [M+H]+) were detected in the tendam mode. Complete system control and data evaluation were done using the Xcaliba software. Values are expressed as picomoles per millilitre of plasma.

Radioimmunoassay (RIA): CGRP contents in plasma and tissue incubating buffer were measured using a rabbit antirat CGRP RIA kit (Phoenix Pharmaceuticals) according to the manufacturer's protocol (Wang and Wang, 2006). This antibody has 100% cross-reactivity with rat α-CGRP and 79% with rat β-CGRP. There is no cross-reactivity with rat amylin, calcitonin, somatostatin, or substance P.

Western blot analysis: Membrane protein of the mesenteric arteries was extracted, separated on a 10% sodium dodecyl sulfate-polyacrylamide gel, and transferred to a polyvinylidene difluoride membrane as described previously (Wang et al., 2005). The membranes were blocked 1 hour at room temperature or overnight at 4oC in 5% milk washing solution (50 mmol/L Tris-HCl, 100 mmol/L NaCl, and 0.1% Tween-20 at pH 7.5). Subsequently, the membranes were incubated with rabbit anti-rat calcitonin receptor-like receptor (CRLR) antiserum (1:5000, Alpha Diagnostic International) or rabbit anti-human receptor activity-modifying protein 1 (RAMP1) polyclonal IgG (1:1000, Santa Cruz Biotechnology) in blocking solution overnight at 4oC. After being washed, the membranes were incubated with goat anti-rabbit IgG-HRP (1:5000, Santa Cruz Biotechnology) in blocking solution at room temperature for 1 hour. The membranes were developed using enhanced chemiluminescence ECL kit (Amersham Pharmacia Biotech) and exposed to film (Hyperfilm-ECL; Amersham Pharmacia Biotech). The films were scanned and analyzed by using the Image Quantity Program (Scion) to obtain integrated densitometric values. β-actin was used to normalize protein loaded on membranes.

Compounds: CAPZ (Calbiochem) was dissolved in dimethyl sulfoxide (10%, v/v), Tween-80 (10%, v/v), and normal saline. CGRP (Sigma) was dissolved in normal saline. MethA (Sigma) was dissolved in ethanol (10%, v/v), Tween-80 (10%, v/v), and normal saline.

Statistical Analysis Values are expressed as mean ±SE. Differences between 2 groups or before and after treatment were analyzed by using the unpaired or paired Student's t test. The differences among groups were analyzed using one-way ANOVA followed by a Bonferroni's adjustment for multiple comparisons. Comparisons between groups at each experiment time point were determined by the use of two-way ANOVA followed by a Bonferroni's test. Differences were considered statistically significant a†P<0.05.

Results:

High salt intake for 3 weeks did not affect the growth rate considering that body weight showed no significant difference between NS- and HS-treated rats (NS, 289±7 g vs. HS, 280±9 g, p>0.05). After 3-week dietary treatment, baseline MAP was significantly higher in HS-treated rats (117±4 mmHg) compared to NS-treated rats (106±3 mmHg, p<0.05).

To determine whether blockade of the TRPV1 affects blood pressure in rats fed a HS diet, MAP and HR responses to bolus injection of CAPZ (3 mg/kg), a selective TRPV1 antagonist, were examined under the fully awake and unrestrained state. As shown in FIG. 21A, the MAP elevation began immediately after injection of CAPZ, and reached the peak within 2 min after injection in rats fed a HS diet. The pressor action of CAPZ lasted for about 6 to 7 min.

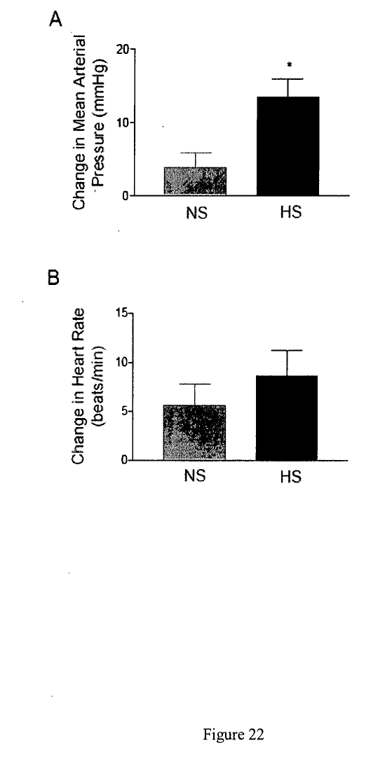

As shown in FIG. 22A, the peak MAP responses to CAPZ injection were significantly increased in HS-treated rats (13±3 mmHg) compared with the NS-treated rats (4±2 mmHg, p<0.05). Therefore, blockade of the TRPV1 leads to a significant increase in blood pressure in rats fed a HS diet but not in rats fed a NS diet, indicating that TRPV1 receptors are activated during HS intake to blunt salt-induced elevation in blood pressure.

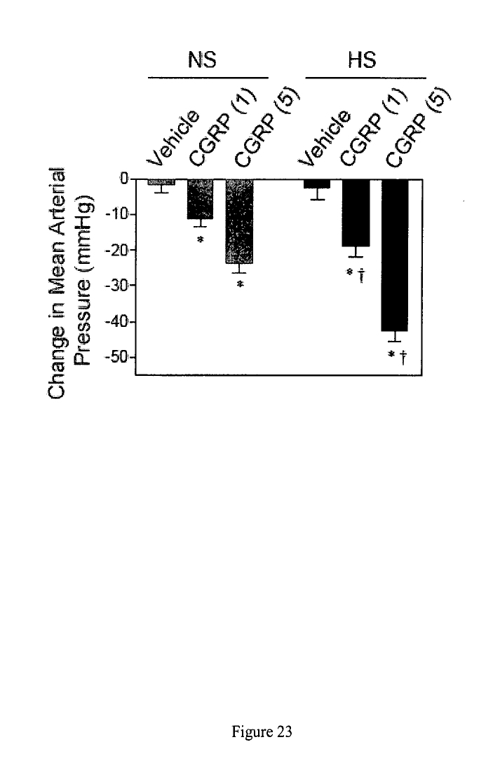

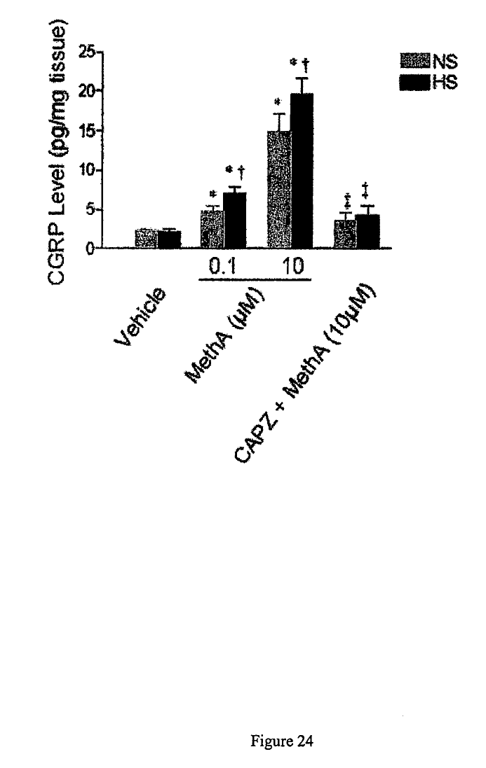

As shown in FIG. 21B and FIG. 22B, no significant change in HR was observed during this experimental period in NS- or HS-treated rats. Effects of CGRP on MAP were examined in conscious rats as shown in FIG. 23. CGRP at the doses of 1 and 5 μg/kg caused dose-dependent reductions in MAP in rats fed a NS and HS diet. The magnitude of the CGRP-induced reductions in MAP in HS-treated rats was significantly greater than that in NS-treated rats (19±3 and 43±3 mmHg in HS-treated rats vs. 11±3 and 24±3 mmHg in NS-treated rats, respectively, p<0.05). The effects of MethA on CGRP release in mesenteric arteries were examined in vitro as shown in FIG. 24. MethA led to a dose-dependent CGRP release in mesenteric arteries in rats fed a NS or HS rats. The CGRP release induced by MethA (0.1 and 10 μM) was significantly greater in HS-treated rats compared to NS-treated rats (7.08±0.84 and 20.01±2.55 μg/mg tissue in HS-treated rats vs. 4.81±0.75 and 14.89±2.36 pg/mg tissue in NS-treated rats, respectively, p<0.05). Moreover, MethA (10 μM)-induced CGRP release was blocked by CAPZ (10 μM) in NS or HS-treated rats.

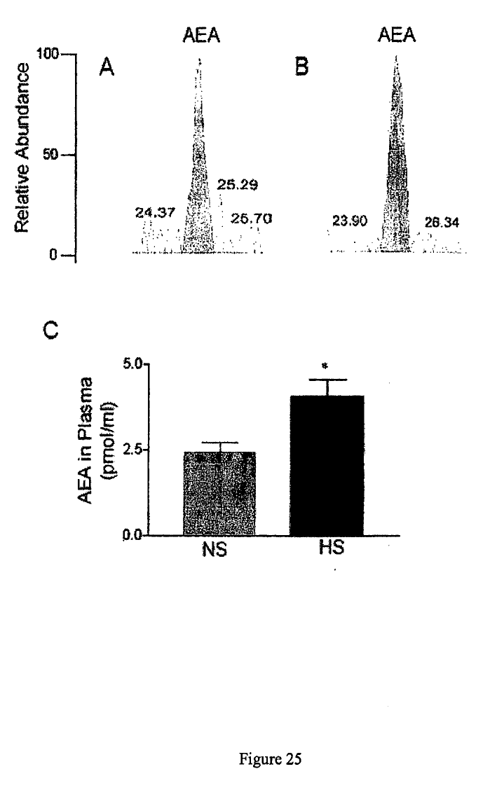

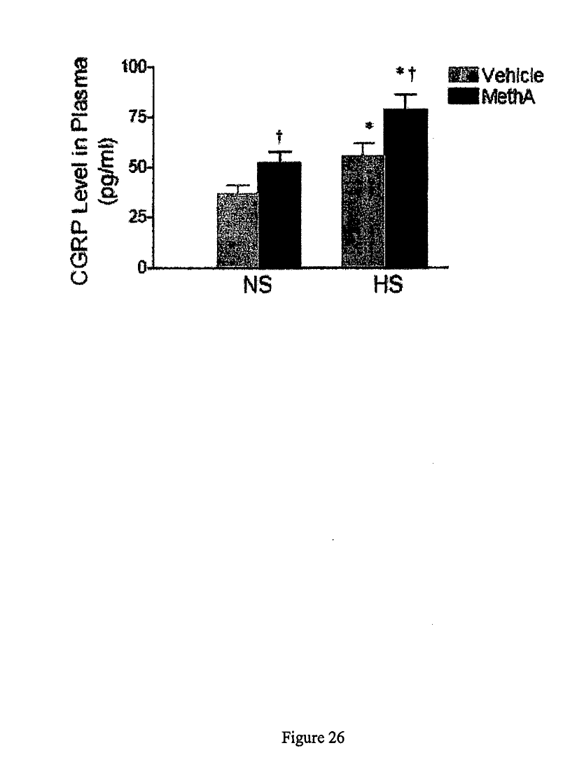

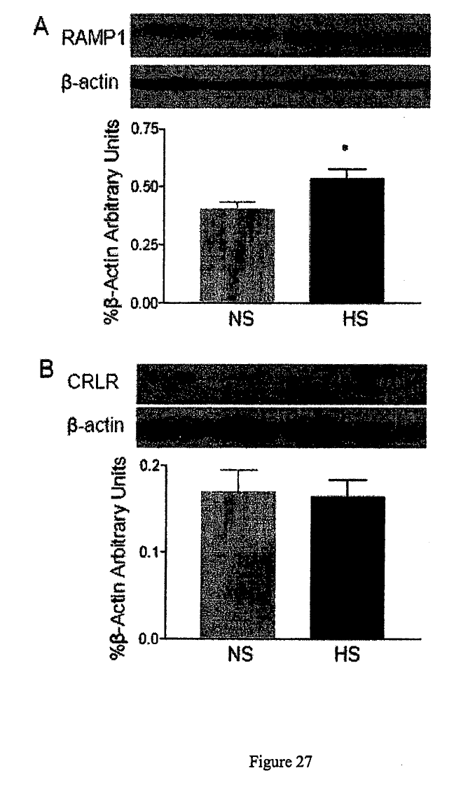

To determine the effect of HS intake on endocannabinoid release, plasma anandamide levels in rats fed a NS or HS rats were assayed by Liquid Chromatography/Electrospray Tandem Mass Spectrometry. HS intake for 3 weeks significantly increased anandamide levels in plasma (NS, 2.40±0.31 μmol/ml; HS, 4.05 ±0.47 pmol/ml, p<0.05) as shown in FIG. 25. The results indicate that the endocannabinoid system was activated during HS intake. To examine the effect of MethA on CGRP release in both NS and HS-treated rats in vivo, plasma CGRP levels were measured by radioimmunoassay (FIG. 26). HS increased plasma CGRP levels (NS, 36.6±3.8 pg/ml; HS, 55.7±6.4 pg/ml, p<0.05). MethA at 5 mg/kg significantly increased plasma CGRP levels in both NS and HS-treated rats (NS, 36.6±3.8 pg/ml vs. 50.4±5.9 pg/ml; HS, 55.7±6.4 pg/ml vs. 78.1±8.5 pg/ml, p<0.05), and MethA induced CGRP release was significantly greater in HS-treated rats compared to NS-treated rats (p<0.05). Western blotting analysis was performed to determine the effect of HS intake on expression of CGRP receptor proteins. Mesenteric RAMP1, one of the key components of the CGRP receptor, was upregulated in HS-treated rats compared to NS-treated rats (0.54±0.04 vs. 0.41±0.03, p<0.05). There was no difference in CRLR expression between the two groups (FIG. 27).

CGRP is one of the most powerful vasodilatory neuropeptides (Wimalawansa, 1996; Kawasaki et al., 1988) via its direct effect or inhibition of sympathetic nervous activity (Ralevic et al., 1995; Oh-hashi et al., 2001). McLatchie et al (McLatchie et al., 1998) have shown that a functional CGRP receptor derives from CRLR but the phenotype is determined by co-expression of a particular RAMPs. RAMPs are required to transport CRLR to the plasma membrane, determining its glycosylation state, and defining its pharmacological properties (McLatchie et al., 1998). Co-expression of PAMPI and CRLR is found to form a CGRP receptor, whereas RAMP2 or RAMP3 co-expressed with CRLR produces an adrenomedullin receptor (McLatchie et al., 1998; Fraser et al., 1999). Our data show that chronic high salt intake causes upregulation of RAMP1 in mesenteric arteries, suggesting that the mechanisms including upregulation and sensitization of the CGRP receptor in the target tissues may also be involved in the enhanced responses to anandamide in rats fed a HS diet. Indeed, our data show that the depressor effect of CGRP is greater in rats fed a HS diet than in rats fed a NS diet. While modulating peripheral vascular reactivity may underlie acute blood pressure regulation, long-term blood pressure regulation is intrinsically linked to renal excretory function (Guyton, 1961; Borst and Borst-de-Geus, 1963). Much of the previous research on salt dependent hypertension has focused on sympathetic nervous system and endocrine regulation of sodium and water balance. In addition to these well investigated systems, sensory nerves and their neurotransmitters facilitate sodium excretion. It was been shown that the kidney is innervated by a dense network of CGRP-positive sensory nerves (Chai et al., 1998). Moreover, CGRP and substance P have direct and indirect effects on tubular ion transport leading to natriuresis and diuresis (Shekhar et al., 1991; Arendshorst et al., 1976). Indeed, several studies have shown that sodium excretion in response to sodium loading is impaired in salt-sensitive hypertension induced by sensory nerve degeneration of neonatally capsaicin treatment or by surgical sensory denervation (Ye and Wang, 2002; Wang et al., 2005; Kopp et al., 2003). In summary, the results shown herein, demonstrate that TRPV1 is activated in rats fed a HS diet, probably owing to elevated anandamide production leading to CGRP release. These effects probably involving altered peripheral vascular reactivity and renal function may play a counterregulatory role in blunting salt-induced increases in blood pressure.

There is increasing evidence showing that HS may contribute to the development of hypertension (Haddy and Pamnani, 1995; Kotchen, 2005). However, mechanisms underlying salt-dependent hypertension are unclear. Although previous studies have shown that CGRP release may play a compensatory role in preventing blood pressure elevation in deoxycorticosterone-salt hypertensive rats (Supowit et al., 1997), the identity of the endogenous ligands triggering enhanced CGRP release remains unclear. Our findings of the current study suggest that elevated anandamide concentrations may attenuate the increase in blood pressure via activation of TRPV1 during HS intake. Thus, it is conceivable that activation of the TRPV1 via enhancement of anandamide production/release or blockade of its metabolic degradation or uptake might be an effective approach for future consideration of new therapeutic strategies in treating hypertension.

### Example VIII

### This Example demonstrates that TRPV1 Gene Knockout Abolishes Preconditioning Protection against Myocardial Injury in Isolated Perfused Hearts in Mice

Although a previous study by the inventors in the previous exampled showed that TRPV1 gene deletion impairs cardiac recovery after ischemic-reperfusion injury (Wang, et al., Circulation 112: 3617-23, 2005; herein incorporated by reference), it was unknown whether TRPV1 would directly mediate PC-induced protection of hearts given that the molecular mechanisms underlying this process are poorly defined. The inventors show herein that TRPV1 expressed in sensory nerves innervating the heart plays a key role in PC-induced protection against myocardial injury and the lack of TRPV1 eliminates such protection, hearts of gene-targeted TRPV1-null mutant (TRPV1−/−) or wild-type (WT) mice were used to determine whether ischemic PC activates the TRPV1, resulting in the release of SP and CGRP to convey the beneficial effects of PC against I/R.

The clinical relevance of these studies relate to the clinical studies in humans showing that patients with angina have a lower in-hospital death rate and a smaller infarct size than patients without angina, suggesting that PC by preinfarction angina might render the myocardium more resistant to infarction from the subsequent prolonged ischemic episode (Tomai et al., Circulation 100: 559-63, 1999; Kloner et al., Circulation 91: 37-47, 1995; all of which are herein incorporated by reference). TRPV1-positive afferent nerves innervating the heart were found to be widely distributed on the epicardial surface of the ventricle (Zahner et al., J Physiol 551: 515-23, 2003; herein incorporated by reference), which can be activated in the setting of myocardial ischemia to mediate the sensation of angina (Benson et al., Ann N Y Acad Sci 940: 96-109, 2001; herein incorporated by reference). Indeed, TRPV1 was shown to integrate and respond to multiple ischemic metabolites, serving as a polymodal detector of pain-producing chemical and physical stimuli (Pan et al., Circulation 110:1826-31, 2004; Tominaga et al., Neuron 21: 531-43, 1998; all of which are herein incorporated by reference). It was shown that PC caused by brief periods of acute myocardial ischemia produces various metabolites including bradykinin (BK), reactive oxygen species (ROS), protons, and arachidonate metabolites (Tjen-A-Looi et al., J Physiol 510: 633-41, 1998; Das et al., Ann NY Acad Sci 874: 49-65, 1999; all of which are herein incorporated by reference). BK, acting on B2 bradykinin receptors, was shown to improve left ventricular function by reducing the incidence of arrhythmias, attenuating myocardial necrosis, and preventing apoptosis after myocardial I/R (Kabir et al., Am J Physiol Heart Circ Physiol 291: H1893-H1899, 2006; Feng et al., Circulation 112: 1-190-5, 2005; all of which are herein incorporated by reference). The inventor's contemplated that BK was exciting sensory nerve endings by activating TRPV1 via production of 12-lipoxygenase metabolites of arachidonic acid, 12-hydroperoxyeicosatetraenoic acid (12-HPETE) which is structurally similar to capsaicin and is the most potent TRPV1 agonist (Shin et al., Proc Natl Acad Sci USA 99:10150-10155, 2002; Chuang et al., Nature 411: 957-962, 2001; all of which are herein incorporated by reference).

ROS, one of the known PC triggers, may activate TRPV1 via the cyclooxygenase pathway of prostaglandin ethanolamides (Ruan et al., J Appl Physiol 101: 644-654, 2006; Van Der Stelt et al., Eur J Biochem 271: 1827-1834, 2004; all of which are herein incorporated by reference). The intracellular mechanism may involve protein kinase C (PKC) given it was shown that PKC plays a pivotal role during PC (Mayr et al., Am J Physiol Heart Circ Physiol 287: H946-H956, 2004; Brooks et al., Circ Res 79: 627-630, 1996; herein incorporated by reference) possibility via mediating BK- or ATP-induced sensitization of the TRPV1 (Sugiura et al., J Neurophysiol 88: 544-548, 2002; Moriyama et al., J Neurosci 23: 6058-6062, 2003; all of which are herein incorporated by reference). Thus, metabolites produced by PC may stimulate cardiac afferent nerves (Pan et al., J Physiol 518: 857-66, 1999; Schultz et al., Cardiovasc Res 38:348-55, 1998; Ruan et al., J Physiol 565: 563-78, 2005; all of which are herein incorporated by reference) through direct or indirect TRPV1 activation leading to depolarization and the release of sensory neurotransmitters such as CGRP and SP.

Indeed, the inventors discovered during the course of developing the present inventions that PC induced protection observed in the WT hearts was suppressed in TRPV1−/− hearts, evident by impaired recovery of the cardiac function as well as higher LDH release in TRPV1−/− hearts with PC comparing to WT hearts with PC. Further, acute blockade of the TRPV1 resulting in complete abrogation of PC-induced cardiac protection, suggesting that no compensatory changes occurred in response to TRPV1 gene deletion. Thus blockade or ablation of the TRPV1 decreases tolerance of the heart to ischemic injury which is lessened by PC under normal conditions such that TRVP1 mediated PC protection against I/R injury in the heart.

Materials and Methods

Langendorff Heart Preparation and Measurements of Cardiac Function

Male TRPV1 gene knockout (TRPV1−/−) strain B6.129S4-TRPV1^{tm1Jul} and matching control wild type (WT) strain C57BL/6J mice were used (Jackson Laboratory, Bar Harbor, Me.). Mice were heparinized (500 U/kg i.p.) and anesthetized with pentobarbital sodium (50 mg/kg i.p.). Hearts from TRPV1−/− and WT mice were cannulated and retrogradely perfuse at 37o C and 80 mmHg with Krebs-Henseleit buffer (118 mmol/L NaCl, 4.7 mmol/L KCl, 1.2 mmol/L MgSO₄, 1.2 mmol/L KH₂PO₄, 2.5 mmol/L CaCl₂, 25 mmol/L NaHCO₃, 0.5 mmol/L Na-EDTA, and 11 mmol/L glucose, saturated with 95% O₂-5% CO₂, pH 7.4) through the aorta in a noncirculating Langendorff apparatus as described previously (Wang et al., Circ Res 93: 776-82, 2003; herein incorporated by reference). A water-filled balloon was inserted into the left ventricle and adjusted to a left ventricular end-diastolic pressure (LVEDP) of 5 to 8 mmHg. The distal end of the catheter was connected to a Digi-Med Heart Performance Analyzer via a pressure transducer. Coronary flow (CF) was continuously measured using an ultrasonic flow probe placed in the aortic perfusion line. Hearts were paced at 400 bpm except during ischemic PC and sustained global ischemia to avoid inducing excessive ventricular tachyarrhythmia during reperfusion, and pacing was reinitiated 2 minutes after reperfusion. Left ventricular developed pressure (LVDP, peak systolic minuses LVEDP) and LV peak positive dP/dt (+dP/dt) during isovolumic contraction were used as indices of LV systolic function; LVEDP and the peak negative dP/dt (−dP/dt) were used as indices of LV diastolic function.

Experimental Protocols:

Hearts were allowed to stabilize for 25 min prior to 3 cycles of 5 minutes of ischemia followed by 5 minutes of reperfusion. Non-preconditioned hearts were time-matched perfused with the same duration as that of preconditioned hearts.

Group 1: Controls. As normal controls (nonischemic), WT and TRPV1−/− hearts were perfused for 130 minutes. For I/R controls, WT and TRPV1−/− hearts were first perfused with Krebs-Henseleit buffer for 55 min (time-matched perfusion of the equilibration period plus the PC period), and subsequently subjected to 30 minutes of no-flow normothermic global ischemia followed by 40-minutes of reperfusion.

Group 2: PC. WT and TRPV1−/− hearts were perfused with Krebs-Henseleit buffer for 25 minutes as the equilibration period and subjected to PC with 3 cycles of 5 minutes of ischemia followed by 5 minutes of reperfusion. The hearts were subjected to 30 minutes of no-flow normothermic global ischemia followed by 40-minutes of reperfusion as that of I/R control groups.

Group 3: PC plus capsazepine (CAPZ). WT and TRPV1−/− hearts were treated with the standard PC protocol as that of group 2 except that CAPZ (10⁻⁷M), a selective antagonist of the TRPV1, was added to the perfusate (at 1% of coronary flow rate) 5 minutes before the PC and continued to perfused for another 5 minutes during the first circle of PC, totaling 10 minutes of perfusion. Two additional concentrations of CAPZ, 10⁻⁶M and 10⁻⁸ M, were used in WT hearts. An additional drug control was added. After a 20-minute equilibration period, WT hearts were perfused with CAPZ (10⁻⁷ M) for 10 minutes without PC, followed by perfusion with Krebs-Henseleit buffer for 25 minutes (time-matched perfusion of the PC period) and then subjected to I/R.

Group 4: PC plus CGRP₈₋₃₇. To determine the role of endogenous CGRP during PC in WT and TRPV1−/−hearts, CGRP₈₋₃₇ (10⁻⁶ M), a selective calcitonin gene-related peptide (CGRP) receptor antagonist, was perfused as that of CAPZ. Two additional concentrations of CGRP₈₋₃₇, 10⁻⁵ M and 10⁻⁷ M, were used in WT hearts. An additional drug control of CGRP₈₋₃₇ was added as that of the CAPZ control group.

Group 5: PC plus RP67580. To determine the role of endogenous SP during PC, hearts from WT and TRPV1−/−mice were perfused as that of CAPZ with RP67580 (10⁻⁷ M), a selective neurokinin-1 (NK1) receptor antagonist. Two additional concentrations of RP67580, 10⁻⁶ M and 10⁻⁸ M, were used in WT hearts. An additional drug control of RP67580 was added as that of the CAPZ control group.

Lactate dehydrogenase (LDH) release: In addition to the measurement of cardiac function, cardiac injury was assessed by measuring LDH release. Perfusion effluent was collected during the first 10 minutes to 20 minutes of I/R and stored at 80° C. until analyzed. Total LDH levels were determined with the use of a CytoTox 96 assay (Promega). The data were expressed as absorbance units released per milliliter per minute per gram of heart wet tissues (Turnbull et al., Am J Physiol Heart Circ Physiol 290: H1103-9, 2006; herein incorporated by reference).

Measurement of SP and CGRP: WT and TRPV1−/−hearts were cut into pieces and put into tubes containing 1.5 ml Krebs-Henseleit buffer that was saturated with 95% O₂-5% CO₂ at 37° C. continuously for 30 min (the stabilization period) (Wang, et al., Circulation 112: 3617-23, 2005). In the PC group, hearts were subjected to 3 cyclic episodes of incubation with Krebs-Henseleit buffer (free of gas and glucose) in an anaerobic chamber and aerated with 100% N to remove residual oxygen for 10 min (an insult that did not induce myocardial cell death as measured by LDH release), followed by normoxic culture in normal glucose Krebs-Henseleit buffer for 10 min. Additional WT and TRPV1−/−hearts were treated the same as that of the PC group except that CAPZ (10⁻⁶ M) was added to the solution. In the normal control group, WT and TRPV1−/−hearts were treated the same as that of the PC group except no deprivation of oxygen and glucose. The samples were purified and analyzed by radioimmunoassay. The assay was performed as recommended by the supplier. Commercially available rat CGRP and SP radioimmunoassay kits (Peninsula Laboratories Inc.) were used for determination of SP and CGRP release which was normalized by the heart weight.

Statistical Analysis Values are expressed as mean ±SEM. Differences among groups with multiple measurements over time were determined by ANOVA (2-way ANOVA) for repeated measurements, and differences between means were identified by the least significant difference test. Comparisons among groups measured at the end of the I/R experiments in the bar charts and in SP, CGRP, and LDH release experiments were performed by 1-way ANOVA analysis followed by the Tukey-Kramer multiple comparison test. The results were considered statistically significant a†P<0.05.

Results:

Although the inventors previously showed that the TRPV1 contributes to cardiac protection during myocardial injury using hearts from TRPV1−/−mice (Wang, et al., Circulation 112: 3617-23, 2005; herein incorporated by reference), no direct evidence was available indicating that TRPV1 directly mediated PC-induced cardiac protection. The inventors' data show herein, that both genetic ablation and acute blockade of TRPV1 by its selective antagonist impairs the PC protective effect. These results provide the first direct evidence that TRPV1 directly mediated cardiac protection induced by PC.

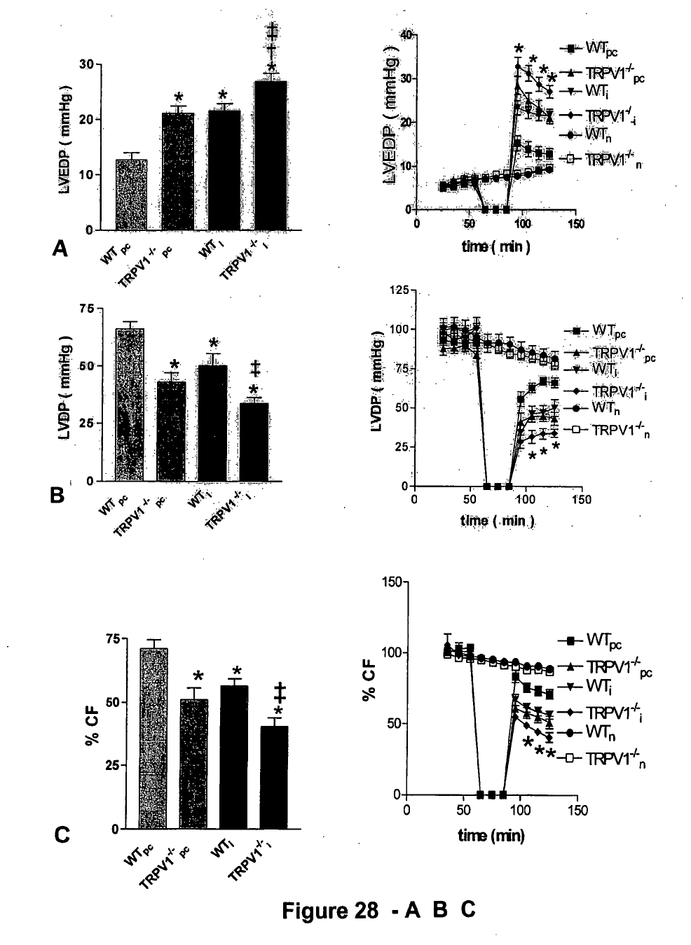

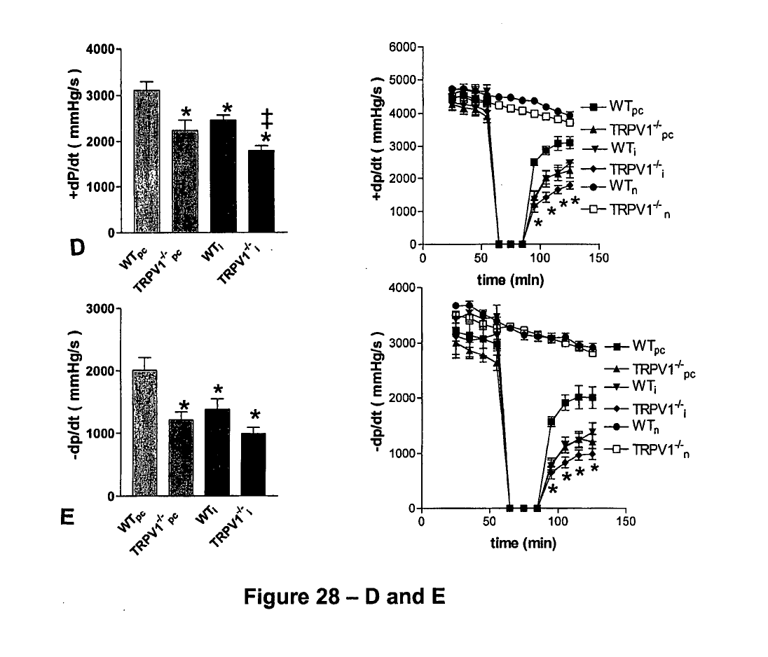

PC protection against I/R injury was impaired in TRPV1−/− hearts. There were no statistically significant differences in hemodynamics between groups under baseline and during PC periods (FIG. 28, right panel). After I/R, PC prevented the increase in LVEDP in WT hearts but not TRPV1−/− hearts, resulting in LVEDP in PC treated TRPV1−/− hearts as high as that of WT hearts without PC. LVEDP increased even further in TRPV1−/−hearts without PC compared to WT hearts without PC and TRPV1−/− hearts with PC. PC enhanced recovery of LVDP, CF, +dP/dt, and −dP/dt in WT hearts but not TRPV1−/−hearts (FIG. 28; right panel). Recovery of LVDP, CF, and +dP/dt was further impaired in TRPV1−/− hearts without PC compared to WT hearts without PC and TRPV1−/−hearts with PC. Thus, hearts from WT showed significantly better preservation of postischemic function by PC whereas PC had less protective effect on hearts from TRPV1−/− mice.

Acute blockade of the TRPV1 abolished PC protection in WT hearts.

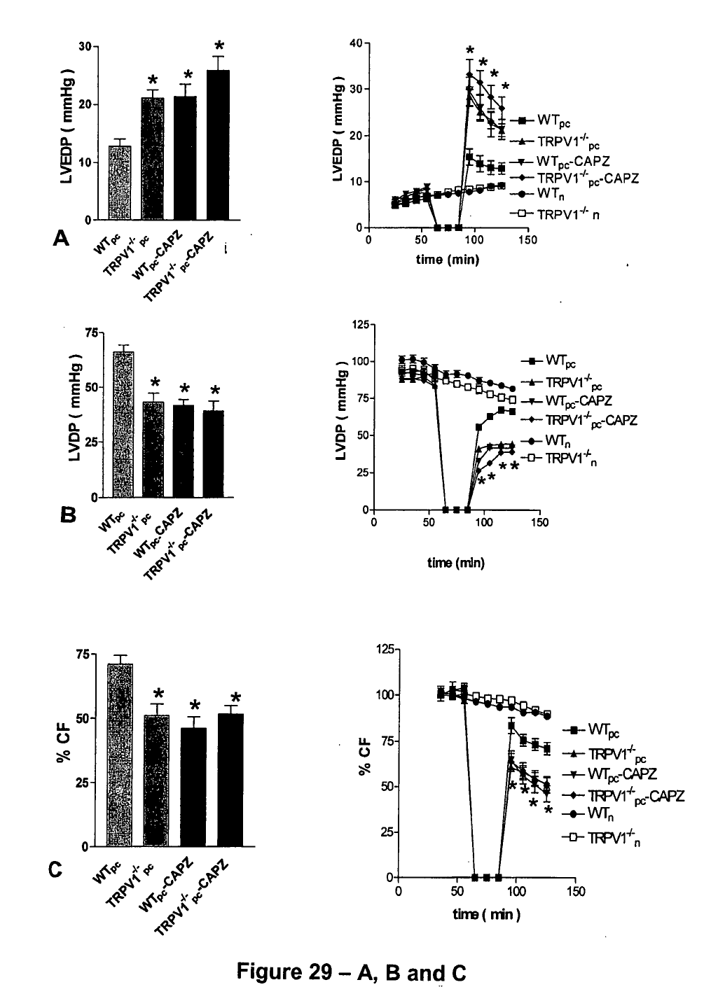

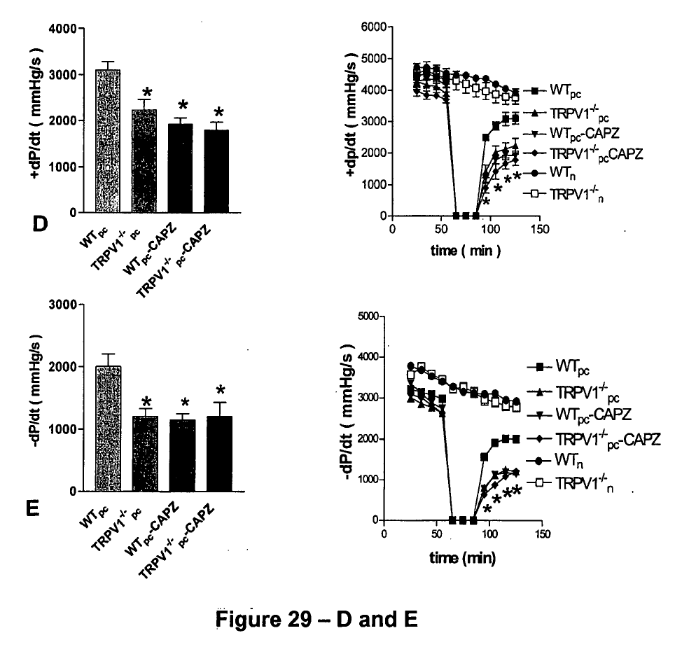

Given that compensatory change may occur in TRPV1−/− mice, the effect of acute blockade of the TRPV1 with its antagonist in WT and TRPV1−/− hearts was examined. Blockade of the TRPV1 with CAPZ abolished PC induced protection by significantly increasing LVEDP and inhibiting recovery of LVDP, CF, +dP/dt, and −dP/dt in WT hearts, but it had no effect on these parameters in TRPV1−/− hearts (FIG. 29, right panel). CAPZ at lower (10⁻⁸ M) or higher (10⁻⁶ M) concentrations also impaired PC protection in WT hearts, with the latter tended to impair more severely (data not shown). CAPZ had no effect on cardiac function in WT hearts without I/R (FIG. 29, right panel). As drug controls, CAPZ giving 25 minutes before I/R had no effect on cardiac recovery after I/R (data not shown).

Blockade of the CGRP Receptor Impaired Pc Protection.

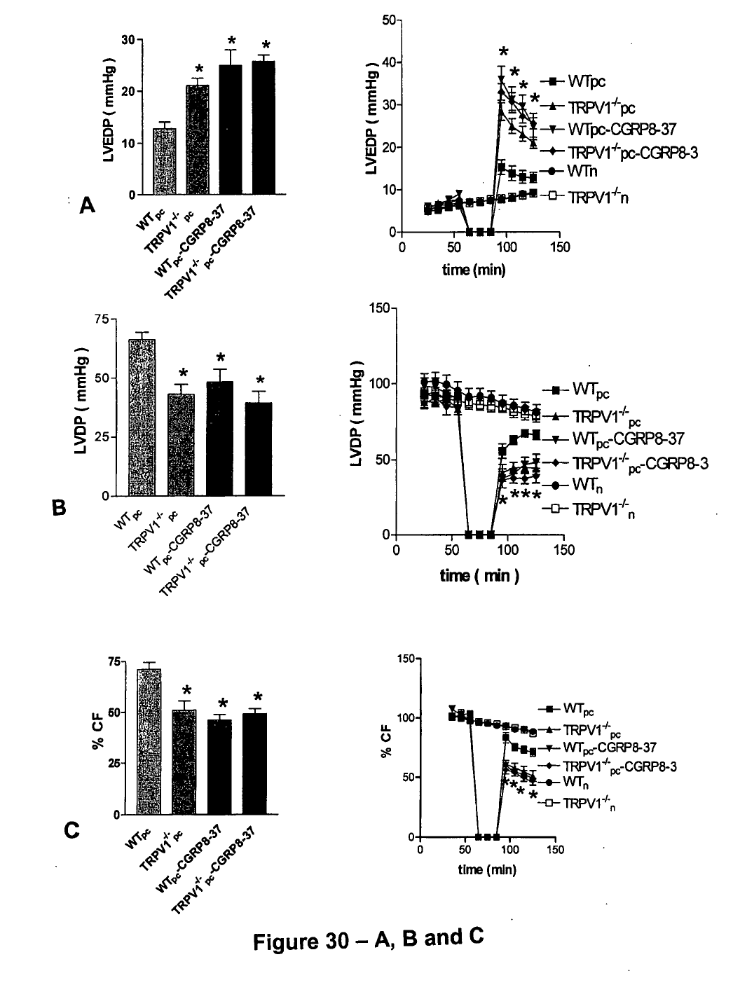

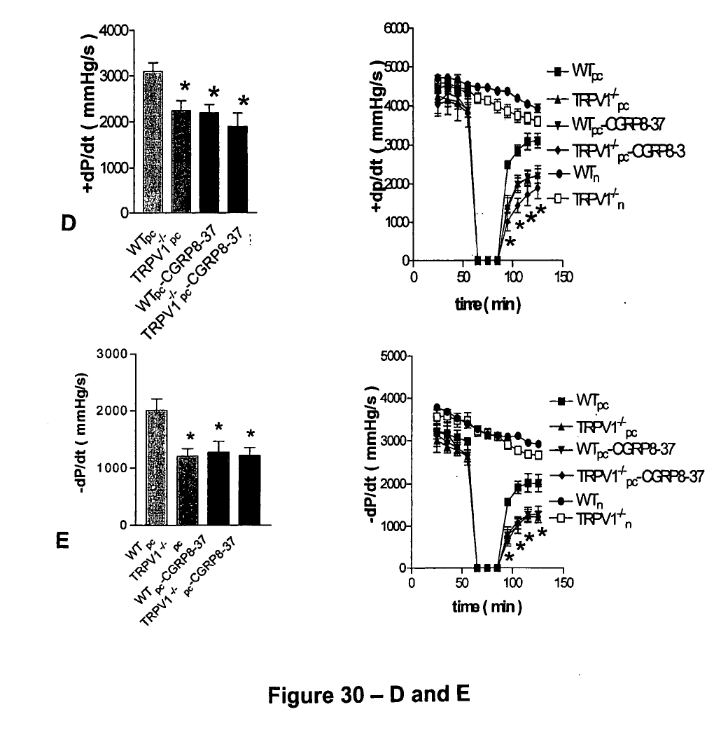

To determine whether endogenous CGRP plays a role in PC-induced cardiac protection, the selective CGRP receptor antagonist, CGRP₈₋₃₇ (10⁻⁶ M) was given before and during PC. CGRP₈₋₃₇ blocked PC-induced cardioprotective effects in WT mice by increasing LVEDP and inhibiting recovery of LVDP, CF, +dP/dt, and −dP/dt in WT hearts, but it had no effect on these parameters in TRPV1−/− hearts (FIG. 30, right panel). Lower (10⁻⁷ M) or higher (10⁻⁵ M) concentrations of CGRP₈₋₃₇ had similar effects on cardiac recovery in WT hearts as that evoked by CGRP₈₋₃₇ at 10⁻⁶ M (data not shown). CGRP₈₋₃₇ (10⁻⁶ M) had no effect on cardiac function in WT hearts without I/R (FIG. 30, right panel). As drug controls, CGRP₈₋₃₇ giving 25 minutes before I/R had no effect on cardiac recovery after I/R.

Blockade of the SP Receptor Impaired Pc Protection.

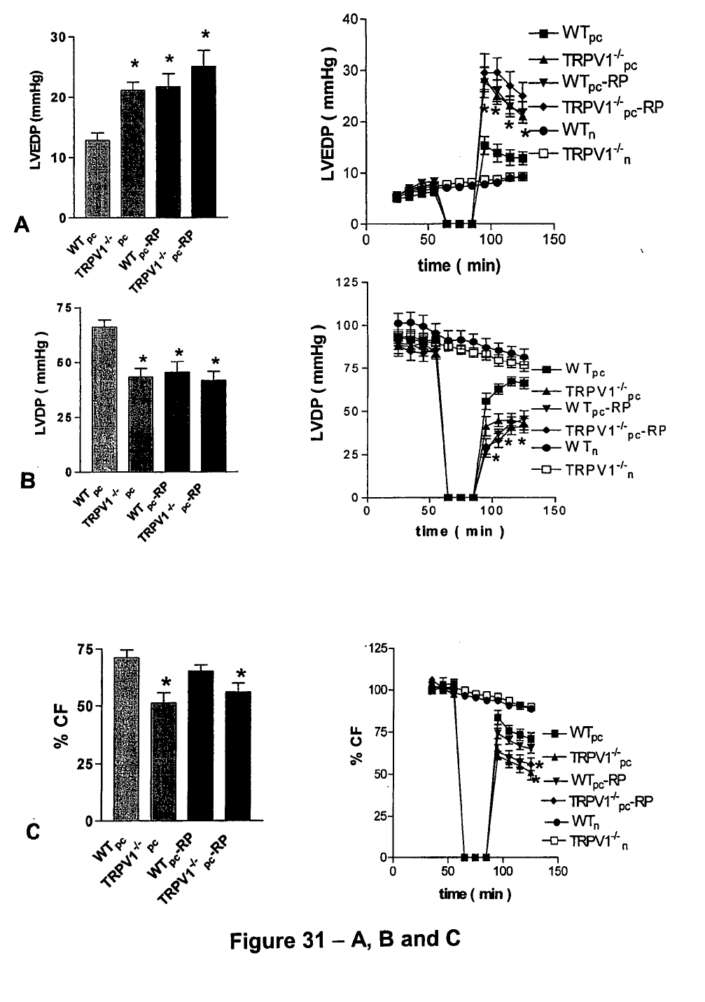

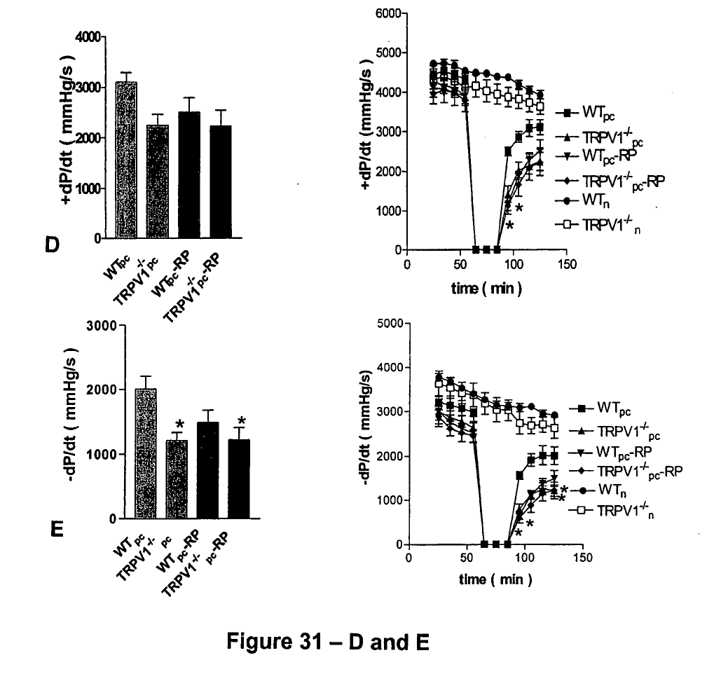

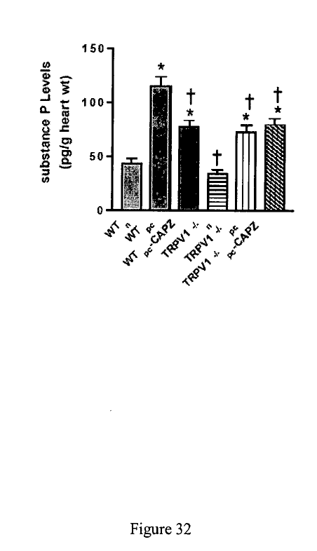

The effect of endogenous SP on PC-inducing cardiac protection was assessed by pretreatment of the hearts with the NK-1 receptor antagonist, RP67580 (10⁻⁷ M). The protective effects of PC were abolished in the presence of RP67580 by increasing LVEDP and decreasing LVDP, +dP/dt and −dP/dt, but it had no effect on these parameters in TRPV1−/− hearts (FIG. 31, right panel). Lower (10⁻⁸M) or higher (10⁻⁶M) concentrations of RP67580 had similar effect on postischemic recovery in WT hearts as that evoked by RP67580 at 10⁻⁷ M (data not shown). RP67580 (10⁻⁷ M) had no effect on cardiac function in WT hearts without I/R, and RP67580 giving 25 minutes before I/R had no effect on cardiac recovery after I/R.

CGRP is one of the most potent vasodilators identified to date in many species (Wang et al., Am J Physiol Heart Circ Physiol 287: H1582-9, 2004; herein incorporated by reference). In addition to vasodilation, CGRP was suggested to play a protective role after myocardial infarction and vascular damage. A wealth of pharmacological data indicates that depletion of CGRP from sensory nerves by prior pre-treatment with capsaicin greatly inhibits or abolishes the protective effect of ischemic PC (Li et al., Eur J Pharmacol 311: 163-7, 1996; Li et al., Eur J Pharmacol 442: 173-7, 2002; Wolfrum et al., Regulatory Peptides 127: 217-24, 2005; all of which are herein incorporated by reference). However, capsaicin induces quite non-specific depletion of neuropeptides from sensory nerve terminals. To avoid this problem, TRPV1−/− hearts combined with selective receptor antagonists were used in the present study. The fact that basal CGRP and SP release is similar in the WT and TRPV1−/− hearts indicates that sensory neuropeptide synthesis and release in TRPV1−/− hearts are not impaired under the resting condition. In contrast, ischemic PC causes increased release of CGRP in WT but not TRPV1−/−hearts. Likewise, blockade of the CGRP receptor impairs the PC protective effects in WT but not TRPV1−/− hearts.

As shown herein, release of SP and CGRP at baseline (normal control) showed no difference between WT and TRPV1−/− hearts. In contrast, SP released from both WT and TRPV1−/− hearts subjected to PC increased remarkably compare to the baseline (P<0.05), but the magnitude of the increase was smaller in TRPV1−/− hearts than in WT hearts (FIG. 31, 32). Furthermore, blockade of the TRPV1 receptor with CAPZ attenuated SP release in WT but not TRPV1−/− hearts subjected to PC, indicates that SP release is partially mediated by the TRPV1 receptor (FIG. 31, 32). CGRP release in WT but not TRPV1−/− hearts subjected to PC increased remarkably compare to the baseline (P<0.05). Furthermore, blockade of the TRPV1 with CAPZ attenuated CGRP release in WT but not TRPV1−/− hearts subjected to PC treatment (FIG. 31, 32).

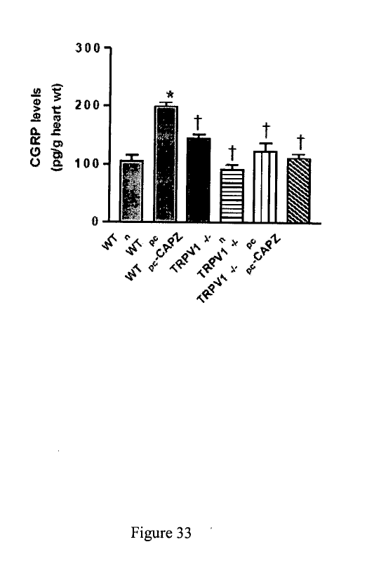

LDH Measurements: LDH levels after I/R were significantly lower in WT hearts with PC than WT hearts without PC and TRPV1−/− hearts with or without PC, and it was lower in TRPV1−/− hearts with PC than without PC (FIG. 33-A), indicating that PC protects hearts in WT but its protection impaired in TRPV1−/− rats. The protective effects of PC in WT hearts were abolished by blockade of TRPV1, CGRP, and NK1 receptors with CAPZ (10⁻ M, FIG. 33-B), CGRP₈₋₃₇ (10⁻⁶M, FIG. 33-C), RP67580 (10⁻⁷M, FIG. 7-D), respectively.

Thus the inventors concluded that ischemic insult capable of inducing TRPV1 activation lead to release of sensory neuropeptides such as SP, CGRP, and other neurokinins from sensory nerve terminals (Manzini et al., Br J Pharmacol. 97:303-12, 1989; Franco-Cereceda et al., Acta Physiol Scand 135:173-87, 1989; all of which are herein incorporated by reference). These neuropeptides produce coronary vasodilation and negative inotropic and chronotropic effects, which would be expected to limit the deleterious consequences of ischemia on the myocardium (Sekiguchi et al., Circulation 89: 366-74, 1994; Bolli et al., Circulation 112: 3541-3, 2005; all of which are herein incorporated by reference).

The inventors further showed that exogenous CGRR and SP added to the perfusion solution before ischemia improved recovery of LVEDP, LVDP, and CF after I/R injury in the mouse hearts, and that beneficial effects of these neuropeptides were evident in WT and TRPV1−/− hearts (Wang, et al., Circulation 112: 3617-23, 2005; herein incorporated by reference). These results indicate that substitution of SP and CGRP prior to I/R is capable of inducing PC-like protection, observations supported by others showing that exogenous or endogenous CGRP and SP have cardiac protection (Wang et al., Am J Physiol Heart Circ Physiol 287: H1582-9, 2004, Li et al., Eur J Pharmacol 311: 163-7, 1996; Ferdinandy et al., Naunyn Schmiedebergs Arch. Pharmacol 1997; 356 (3), 356-63; Ustinova et al., Cardiovasc Res 30: 55-63, 1995; Li et al., Eur J Pharmacol 442: 173-7, 2002; Wolfrum et al., Regulatory Peptides 127: 217-24, 2005; all of which are herein incorporated by reference). Moreover, the facts that PC caused higher SP and CGRP release in WT than TRPV1−/− hearts and acute blockade of the TRPV1 decreased SP and CGRP release in WT hearts indicate that these neuropeptides may account for, at least in part, TRPV1-mediated PC protection.

These examples show that endogenous CGRP released from isolated mouse hearts by activation of TRPV1 by PC would contribute, at least in part, to PC induced cardiac protection.

The mechanisms underlying CGRP-induced PC protection are unclear. However, several possibilities exist. The cardiac protective effects afforded by CGRP-mediated ischemic preconditioning have been suggested to be related to inhibition of cardiac TNF-a production (Li et al., Eur J Pharmacol 442: 173-7, 2002; herein incorporated by reference) to induce an anti-inflammatory effect to prevent postischemic leukocyte rolling and adhesion (Kamada et al., Am J Physiol Heart Circ Physiol 290: H531-7, 2006; all of which are herein incorporated by reference) and to reduce the oxidative stress damages via inhibition of apoptosis due to the I/R sequence (Schaeffer et al., Ann N Y Acad Sci 1010:733-7, 2003; herein incorporated by reference). CGRP also may offer a protection from ischemia by causing microvascular vasodilator by activation of an NO- and endothelium-independent or endothelium-dependent vasodilator pathway (Brain et al., Physiol Rev 84: 903-34, 2004; herein incorporated by reference).

It was shown that SP is co-localized with other sensory neuropeptides especially CGRP and neurokinin A in sensory nerve terminals (Lundberg et al., Eur J Pharmacol 108:315-9, 1985; herein incorporated by reference) and is released from cardiac afferent fibers during myocardial ischemia to protect the heart from I/R injury (Hua et al., Am J Physiol Heart Circ Physiol 286: H1654-64, 2004; Ustinova et al., Cardiovasc Res 30: 55-63, 1995; all of which are herein incorporated by reference). In the present study, the inventors found that PC increased SP release in both WT and TRPV1−/− hearts, although the magnitude of the increase was smaller in TRPV1−/− hearts than in WT hearts. Also, blockade of the NK1 receptor impaired PC-induced protection in WT but not TRPV1−/− hearts for providing additional information on TRPV mediated effects (Wang, et al., Circulation 112: 3617-23, 2005; herein incorporated by reference) by showing that harmful I/short term and non-lethal PC increase SP release, and that TRPV1 activation is responsible for, at lease in part, PC-induced SP release. The cardiac protective effect of SP is possibly mediated by nitric oxide release leading to vasodilatation of coronary arteries (Christie et al., Br J Pharmacol 98: 397-406, 1989; Burnstock, J Hypertens Suppl 8: S95-106, 1990; all of which are herein incorporated by reference). However, the mechanisms underlying the protective effect of SP on the myocardium cannot be fully explained by coronary vasodilatation. The data in the present study showed that RP67580 increased LVEDP and decreased LVDP, +dP/dt, and −dP/dt but had no effect on CF in WT hearts. These results indicate that protective effects of SP may not depend solely upon improved total perfusion of the heart, rather, it may affect the distribution of regional myocardial flow and thus produces its beneficial effect without altering total cardiac flow. It was shown that SP increases NO synthesis (Burnstock, J Hypertens Suppl 8: S95-106, 1990; herein incorporated by reference), which acts as a second messenger resulting in activation of protein kinase C and tyrosine kinase phosphatidylinositol 3-kinase as well as open of the K_{ATP} channel (Dawn et al., Ann NY Acad Sci 962:18-41, 2002; herein incorporated by reference). Moreover, NK-1 activated by SP stimulates cyclooxygenase-2 and prostaglandin E₂ expression (Koon et al., Immunology 176: 5050-59, 2006; herein incorporated by reference). These may mediate SP-induced PC protect of the heart (Shinmura et al., PNAS 97:10197-20, 2000; herein incorporated by reference).

In summary, the experimental information presented herein provide direct evidence that the TRPV1 mediated ischemia PC via at least in part increasing endogenous CGRP and SP. Given that sensory nerve function is impaired in many pathological conditions, such as diabetes and aging in which ischemic PC was shown to be attenuated, (Kersten J et al., Am J Physiol Heart Circ Physiol 278: H1218-24, 2000; Tanaka et al., Am J Physiol Heart Circ Physiol 282: H2018-H2023, 2002; Tani et al., Circulation 95: 2559-66, 1997; Bartling et al., Ann Thora Surg 76:105-11, 2003; Wu et al., J Thorac Cardiovasc Surg 122: 972-8, 2001; all of which are herein incorporated by reference) the inventors discoveries are contemplated to provide new compounds and provide additional compounds for providing a molecular basic for treatment of these pathological conditions based at least in part on TRPV1 receptor activation and downstream mediators.

### Example IX

Objective: To determine the function and regulation of thiazide-sensitive NaCl co-transporters (NCC), Na†K†2Cl co-transporters (NKCC2), and epithelial sodium channels

Design and methods Neonatal Wistar rats were treated with capsaicin or vehicle. Seven-week-old male rats were treated for 2 weeks with: vehicle plus a normal (Con-NS) or high (Con-HS) sodium diet, and capsaicin pretreatment plus a normal (Cap-NS) or high (Cap-HS) sodium diet. Mean arterial pressure (MAP), renal excretory function, and protein expression determined by Western blot were performed.

Results MAP was increased in Cap-HS compared with other groups. Trichlormethiazide increased urine sodium excretion (UNaV) and urine flow rate (UFR) and decreased MAP in Cap-HS rats only. Furosemide increased UnaV and UFR in Cap-NS, Con-HS and Cap-HS, and decreased MAP in Cap-HS rats only. Amiloride had no effect on UNaV, UFR and MAP in any group. Renal NCC contents were increased in Cap-HS compared with Con-NS, Con-HS and Cap-NS rats, and NKCC2 expression was increased in Cap-NS, Con-HS and Cap-HS compared with Con-NS rats. No change was found in ENaC alpha subunit expression. The capsaicin-induced release of calcitonin gene-related peptide from renal tissues was decreased in

Results:

At the end of 2 weeks' dietary treatment, MAP was significantly increased in Cap-HS (129_{—}2 mmHg) compared with Cap-NS, Con-NS and Con-HS rats. 2 mmHg, respectively; n¼16, P<0.001). These results were consistent with previous findings. Hematocrit was significantly lower in Cap-H Con-HS rats (0.449_{—}0.002, 0.447_{—}0.002 and 0.450_{—}0.005, respectively; n¼5, P<0.001), suggesting that the circulating fluid volume was increased in Cap-HS rats as a result of sodium and water retention. Intravenous injections of diuretics caused increases in the UFR. The UFR reached a peak at 10, 5, and 10 min after the injection of trichlormethiazide, furosemide, and amiloride, respectively. The UFR returned almost completely to the baseline at 30 min after the injection of these diuretics.

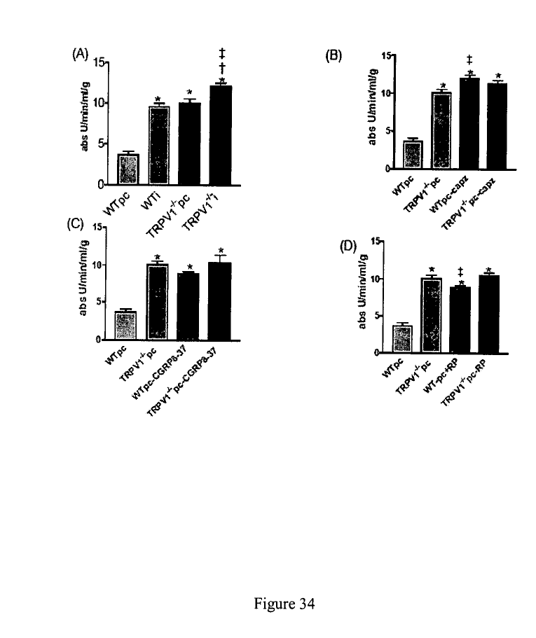

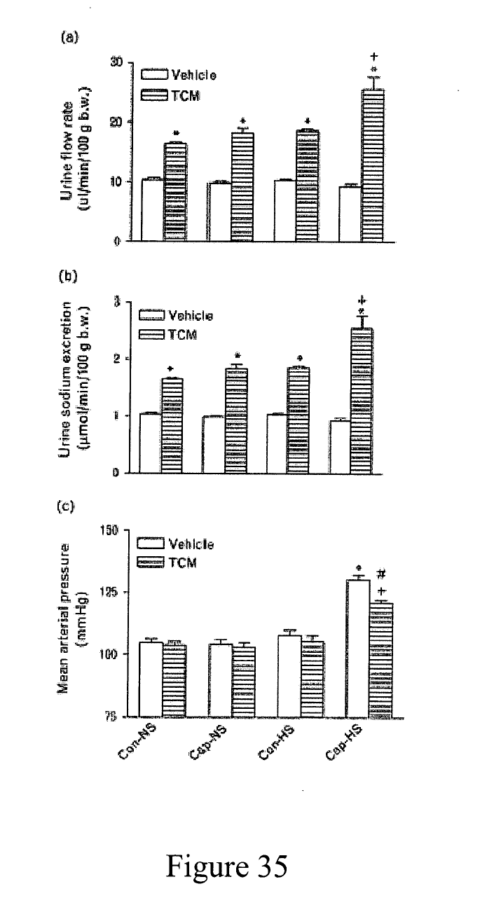

Blockade of NCC with trichlormethiazide significantly increased urine flow/sodium excretion rates in all groups, and the increases were more profound in Cap-HS compared with Cap-NS, Con-NS, and Con-HS rats (n¼5, P<0.01; FIG. 34). Baseline MAP was significantly increased in Cap-HS rats only, and trichlormethiazide decreased MAP in Cap-HS but not in Cap-NS, Con-NS, and Con-HS rats (n¼5, P<0.001; FIG. 35).

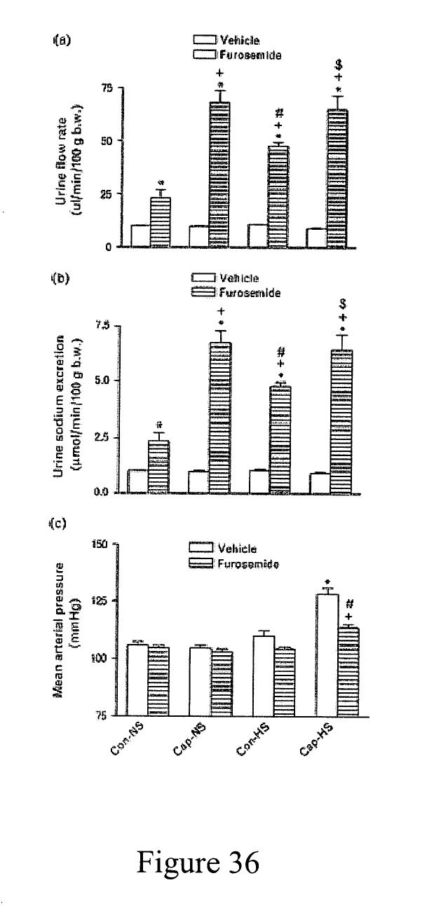

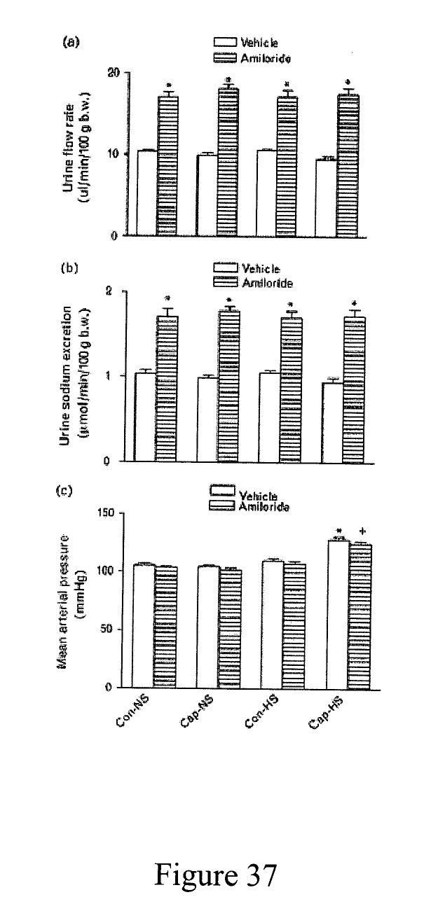

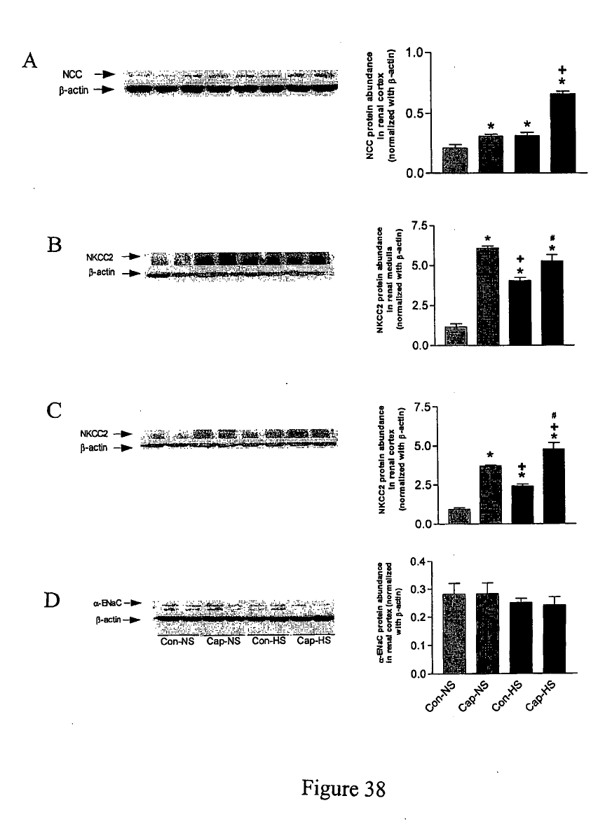

Blockade of NKCC2 with furosemide significantly increased urine flow/sodium excretion rates in all groups (FIG. 35). These increases were much greater in Cap-HS and Cap-NS rats compared with Con-HS and Con-NS rats, and also when Con-HS were compared with Con-NS rats (n¼6, P<0.05; FIG. 36). Likewise, baseline MAP was significantly increased in Cap-HS rats only, and furosemide decreased MAP in Cap-HS but not in Cap-NS, Con-NS, and Con-HS rats (n¼6, P<0.01; FIG. 36). Blockade of ENaC with amiloride significantly increased the urine flow/sodium excretion rates in all groups of rats without distinction (FIG. 37). Baseline MAP was significantly increased in Cap-HS rats only, but amiloride had no effect on MAP in any group (n¼5, P>0.05; FIG. 36). Western blot analysis showed that the abundance of NCC in the renal cortex was significantly increased in Cap-HS compared with Cap-NS, Con-NS and Con-HS rats (n¼5, P<0.01), and was higher in Cap-NS and Con-HS rats than in Con-NS rats (P<0.05; FIG. 38). In the renal medulla, NKCC2 abundance was significantly increased in Cap-HS and Cap-NS rats compared with Con-NS and Con-HS rats (n¼5, P<0.05 FIG. 38), and was higher in Con-HS than in Con-NS rats (P<0.01). In the renal cortex, NKCC2 abundance was significantly increased in Cap-HS compared with Cap-NS, Con-NS and Con-HS rats (n¼5, P<0.05), and was higher in Cap-NS and Con-HS than in Con-NS rats (P<0.01; FIG. 37). There was no difference between groups in alpha-ENaC abundance in the renal cortex (n¼5, P>0.05; FIG. 38).

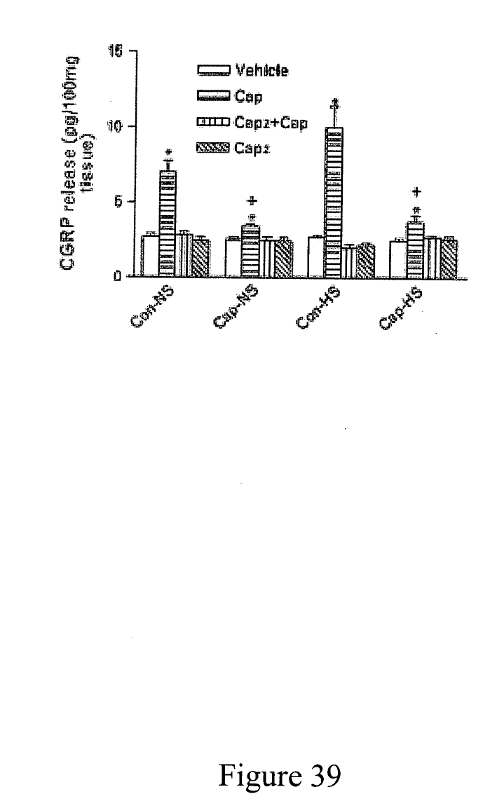

To examine renal sensory nerve function, renal CGRP release after stimulation of the renal sensory nerves with capsaicin was examined. Renal CGRP release in control rats was dose-dependent in response to capsaicin (0.1, 1 and 10 mmol/l; CGRP 2.66_{—}0.15, 10.95_{—}1.23 and 14.39_{—}1.22 pg/100 mg tissue, respectively; n/44, P<0.05). Renal CGRP release in response to 1 mmol/l capsaicin was significantly lower in Cap-HS and Cap-NS than in Con-NS and Con-HS rats (n¼5, P<0.001; FIG. 39), suggesting that renal sensory nerve function was impaired in capsaicin-treated rats. Capsazepine at 10 mmol/l blocked the effect of capsaicin on stimulating CGRP release in all groups (FIG. 39).

### Example X

Enhanced oxidative stress in kidneys of salt-sensitive hypertension: role of sensory nerves (Am J Physiol Heart Circ Physiol 291: H3136-H3143, 2006). To determine the mechanism(s) underlying enhanced oxidative stress in kidneys of salt-sensitive hypertension, neonatal Wistar rats were given vehicle or capsaicin (CAP, 50 mg/kg sc) on the first and second days of life. After being weaned, male rats were assigned into four groups and treated for 2 wk with the following: vehicle+a normal sodium diet (NS, 0.4%, CON-NS), vehicle+a high-sodium diet (HS, 4%, CON-HS), CAP+NS (CAP−NS), and CAP+HS (CAP−HS). Systolic blood pressure was significantly increased in CAP−HS but not CAP−NS or CON-HS rats. Plasma and urinary 8-iso-prostaglandin F2alpha levels increased by approximately 40% in CON-HS and CAP−HS rats compared with their respective controls fed a NS diet (P≦0.05), and these parameters were higher in CAP−HS compared with CON-HS rats. Superoxide (O2—) levels in the renal cortex and medulla increased by approximately 45% in CAP−HS compared with CON-HS, CON-NS, and CAP−NS rats (P≦0.05).

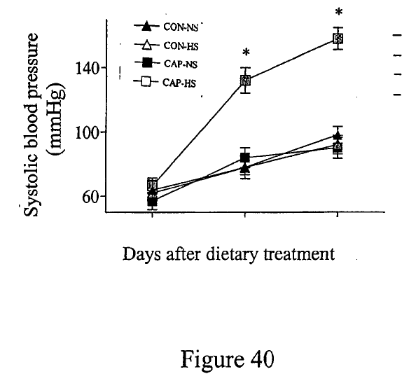

Enhanced O2— levels in the cortex and medulla in CAP−HS rats were prevented by preincubation of renal tissues with apocynin, a selective NAD(P)H oxidase inhibitor. Protein expression of NAD(P)H oxidase subunits, including p47phox and gp92phox in the renal cortex and medulla, was significantly increased in CAP−HS compared with CON-HS, CON-NS, and CAP−NS rats. In contrast, protein expression and activities of Cu/Zn SOD and Mn SOD were significantly increased in the renal medulla in both CAP−HS and CON-HS but in the cortex in CAP−HS rats only. Creatinine clearance decreased by_{—}45% in CAP−HS rats compared with CON-HS, CON-NS, and CAP−NS rats (P ≦0.05). O2— levels in the renal cortex of CAP−HS rats negatively correlated with creatinine clearance (r=−0.76; P≦0.001). Therefore, regardless of enhanced SOD activity to suppress oxidative stress, increased oxidative stress in the kidney of CAP-treated rats fed a HS diet is likely the result of increased expression and activities of NAD(P)H oxidase, which may contribute to decreased renal function and increased blood pressure in these rats. Our results suggest that sensory nerves may play a compensatory Tail-cuff systolic blood pressure was significantly higher in the CAP−HS group compared with CON-HS, CON-NS, and CAP−NS groups (FIG. 40). On day 12 after the dietary treatment, tail-cuff systolic blood pressure was significantly increased in CAP−HS rats (158 7 mmHg, P<0.05) compared with CON-NS (98 6 mmHg), CON-HS (92 6 mmHg), and CAP−NS (90 5 mmHg) rats. Thus neonatal treatment with CAP did not increase blood pressure in rats fed a NS diet but led to an elevation in blood pressure in rats fed a HS diet.

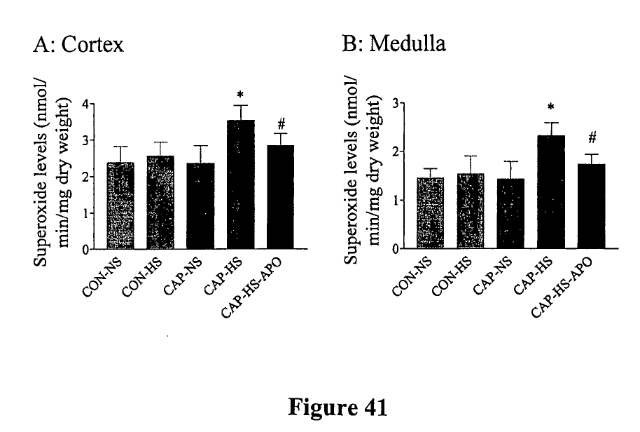

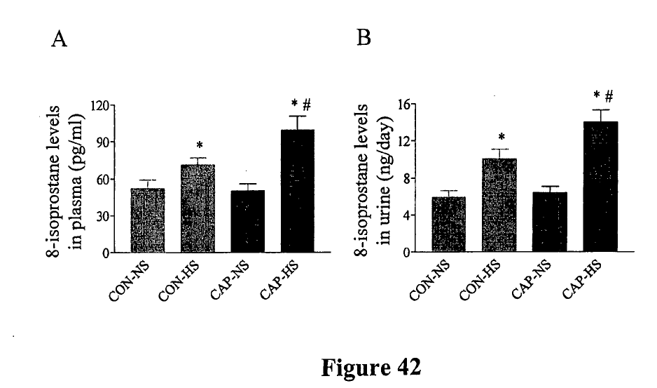

As shown in FIG. 41, HS intake for 2 wk significantly increased renal cortical and medullary O₂— production (nmol_min_{—}1_mg dry wt_{—}1) in CAP−HS rats (3.53 0.46; 2.32 0.29, P_{—}0.05) compared with CON-NS (2.37 0.49; 1.45 0.21), CON-HS (2.55 0.43; 1.54 0.39), and CAP−NS (2.35 0.55; 1.43 0.40) rats. Ex vivo incubation of the renal cortex and medulla with apocynin (0.1 mM), a selective NAD(P)H oxidase inhibitor, significantly reduced O₂— production in the renal cortex and medulla in CAP−HS rats (cortex, 3.53 0.46 vs. 2.83 0.38; medulla, 2.32 0.29 vs. 1.73 0.23, P<0.05). In contrast, apocynin had no significant effects on O₂— production in CON-HS, CON-NS, and CAP−NS rats. The results suggest that increased NAD(P)H oxidase activity is the source of increased O₂— production in renal tissues in CAP−HS rats. Plasma and urine 8-isoprostane, an index of oxidative stress, are shown for vehicle or CAP-treated rats fed a NS or HS diet in FIG. 42. HS intake alone produced a significant increase in plasma 8-isoprostane levels (71.2 6.5 pg/ml) and urine 8-isoprostane excretion (10.1 1.1 ng/day) compared with CON-NS rats (52.5 7.3 pg/ml; 5.9 0.8 ng/day). Moreover, CAP treatment did not increase plasma and urine 8-isoprostane levels in rats fed a NS diet but caused an further elevation in plasma and urine 8-isoprostane levels in rats fed a HS diet (99.2 4.9 pg/ml; 14.0 1.4 ng/day) compared with CON-HS rats (71.2 6.5 pg/ml; 10.1 1.1 ng/day, P<0.05).

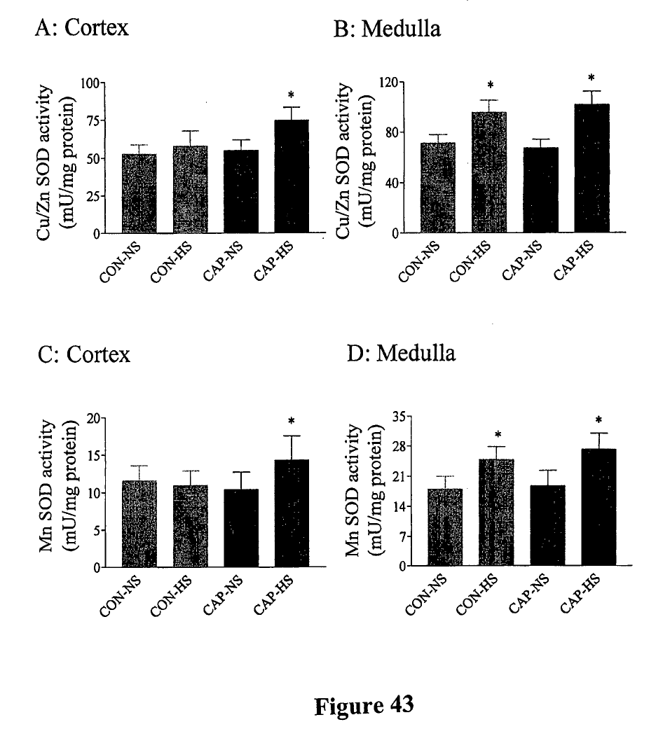

FIG. 43 shows that, 2 wk after initiation of dietary treatment, the renal cortical Cu/Zn SOD and Mn SOD activity (in mU/mg protein) were increased in CAP−HS rats (74.6 9.6; 14.3 3.5, P<0.05) compared with CON-NS (52.7 6.6; 11.6 2.2), CON-HS (57.8 10.8; 10.9 2.1), and CAP−NS (54.8 7.6; 10.4 2.5) rats. In contrast, HS intake significantly increased renal medullary Cu/Zn SOD and Mn SOD activity in both vehicle (95.6 10.8; 24.8 3.3) and CAP (101.7 11.6; 27.1 4.2)-treated rats compared with vehicle (71.2 7.4; 17.9 3.2) and CAP (67.0 7.6; 18.7 3.8) (P<0.05)-treated rats fed a NS diet. There were no differences in renal medullary Cu/Zn SOD and Mn SOD activities between CON-HS and CAP−HS rats.

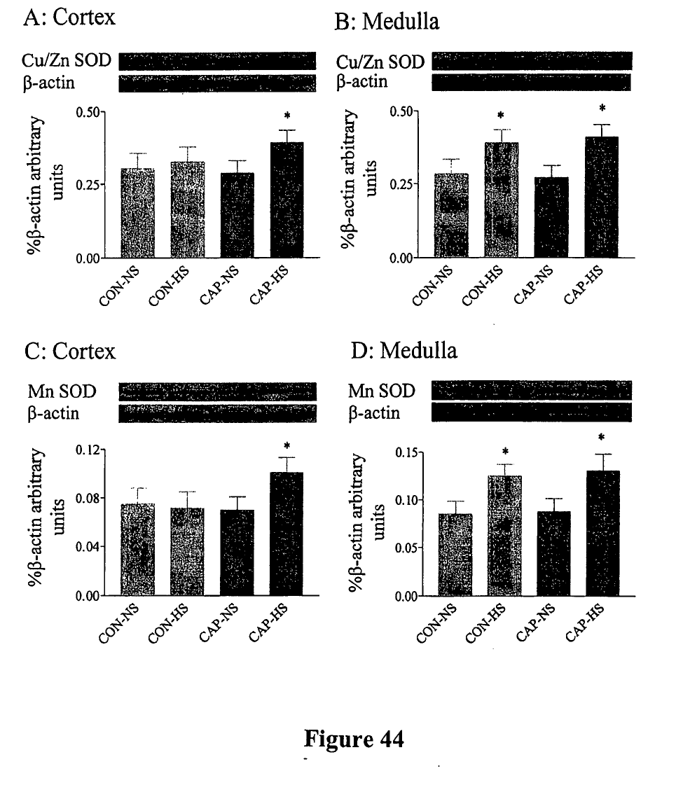

Consistent with the changes in renal Cu/Zn SOD and Mn SOD activity, the renal cortical Cu/Zn SOD and Mn SOD protein expression (%-actin arbitrary units) were increased in CAP−HS (0.39 0.05; 0.10 0.01, P<0.05) compared with CON-NS (0.30 0.06; 0.08 0.02), CON-HS (0.33 0.06; 0.07 0.02), and CAP−NS (0.29 0.05; 0.07 0.01) rats (FIG. 44). Also, HS intake increased renal medullary Cu/Zn SOD and Mn SOD protein expression in both vehicle (0.39 0.05; 0.13 0.01) and CAP (0.41 0.05; 0.13 0.02) (P≦0.05)-treated rats compared with vehicle (0.29 0.06; 0.09 0.02) and CAP (0.27 0.05; 0.09 0.02)-treated rats fed a NS diet.

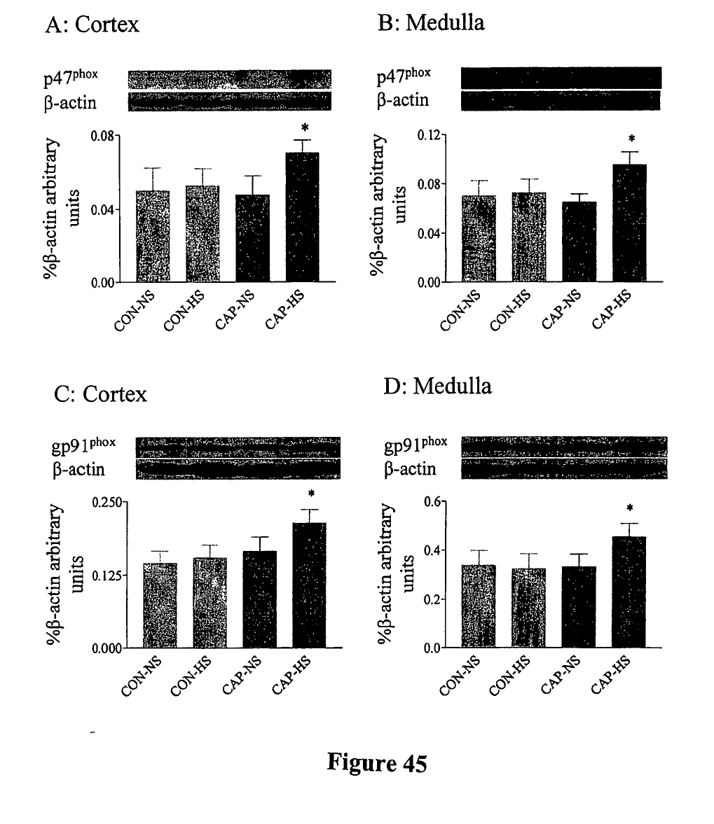

FIG. 45 shows the renal protein expression of NAD(P)H oxidase subunits, including p47phox and gp91phox. A HS loading for 2 wk increased renal cortical and medullary p47phox protein expression in CAP-treated rats (0.07 0.01; 0.1 0.01 arbitrary units, P≦0.05) compared with CAP−NS rats (0.05 0.01; 0.07 0.01 arbitrary units). Similar to p47phox protein expression, renal cortical and medullary gp91 phox protein expression was increased in CAP−HS (0.21 0.03; 0.45 and 0.06 arbitrary units, P<0.05) compared with CAP−NS rats (0.17 0.03; 0.33 0.60 arbitrary units). In contrast, a HS loading did not increase renal cortical and medullary p47phox and gp91 phox protein expression in vehicle-treated rats.

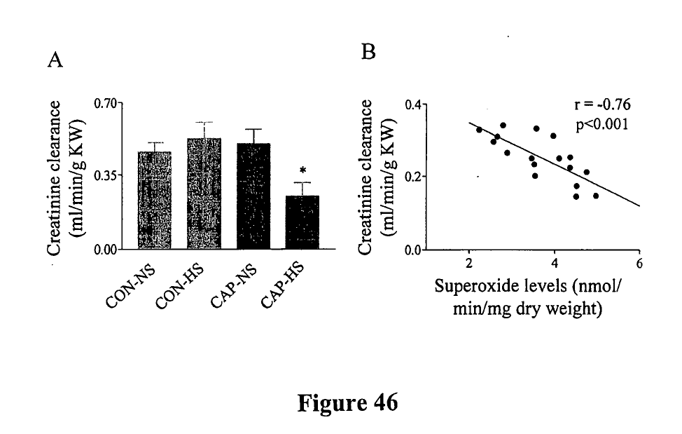

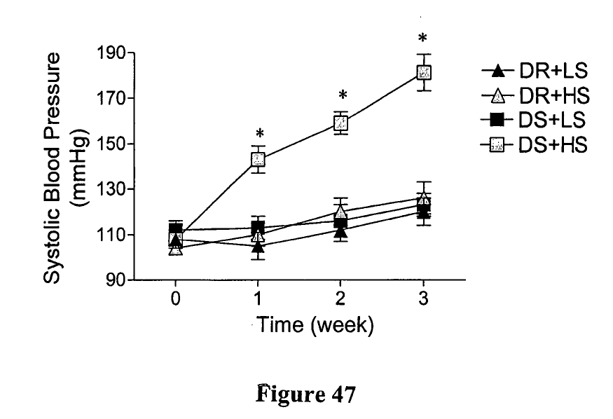

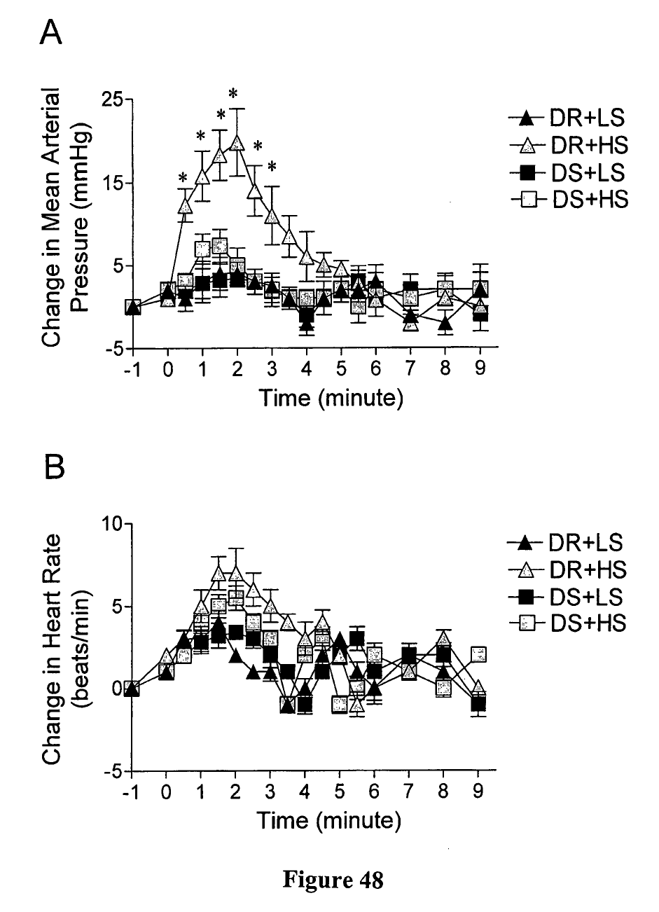

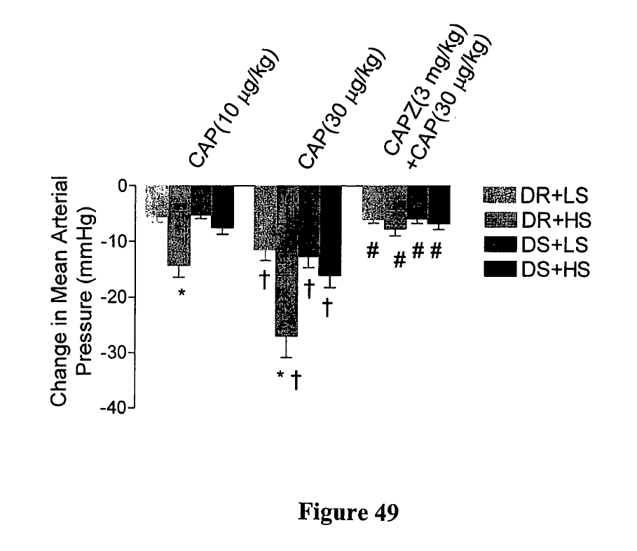

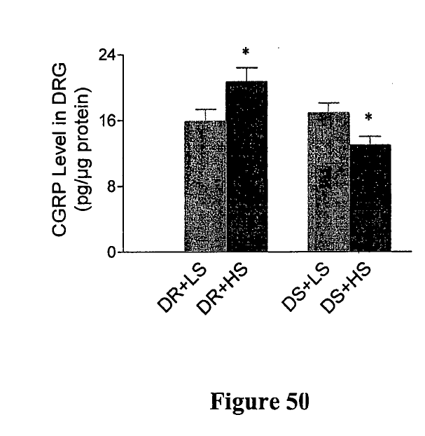

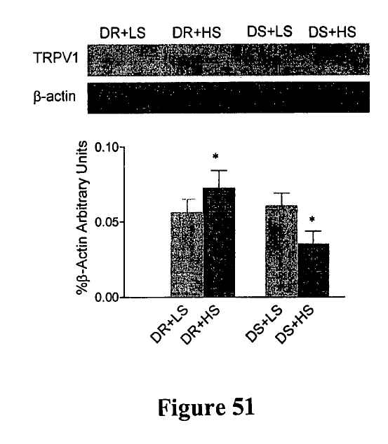

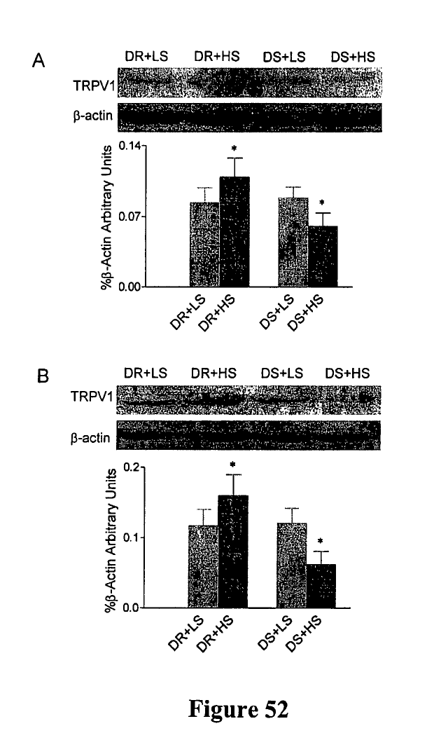

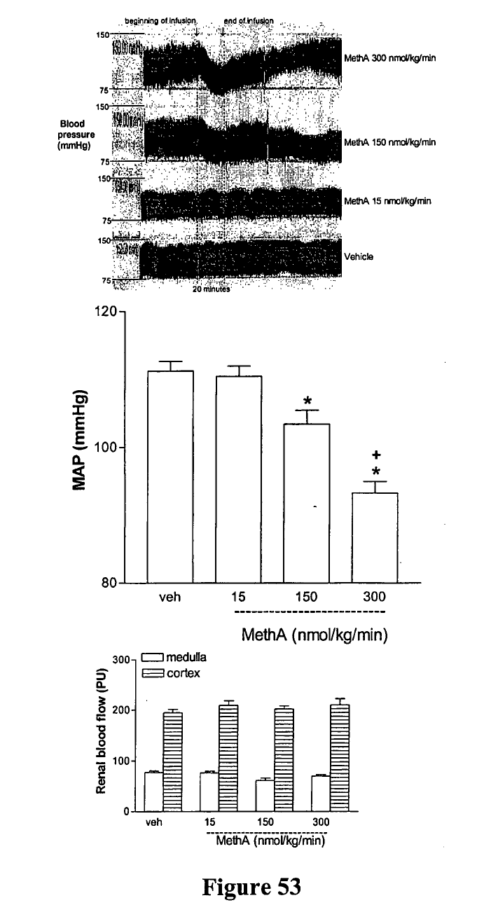

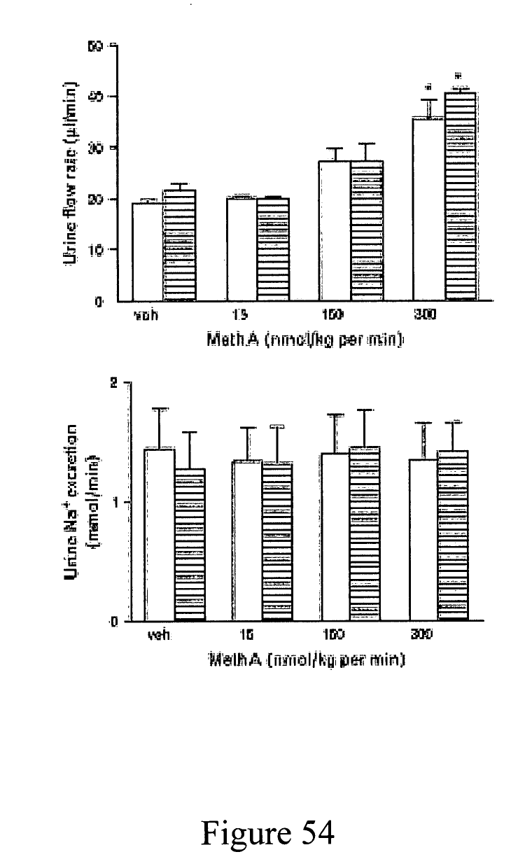

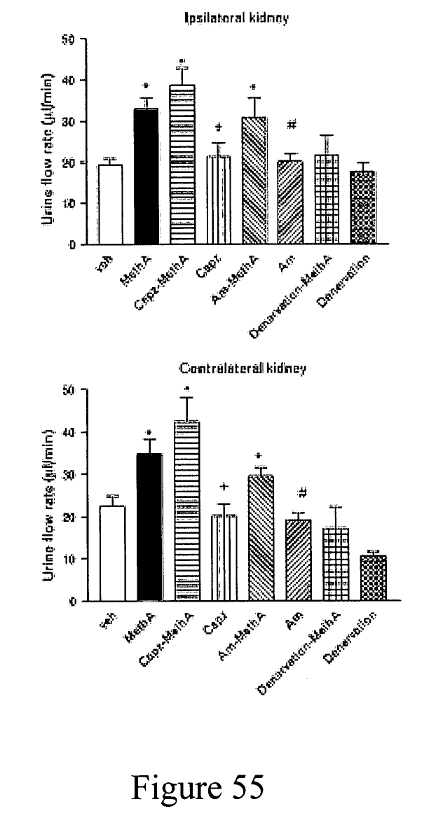

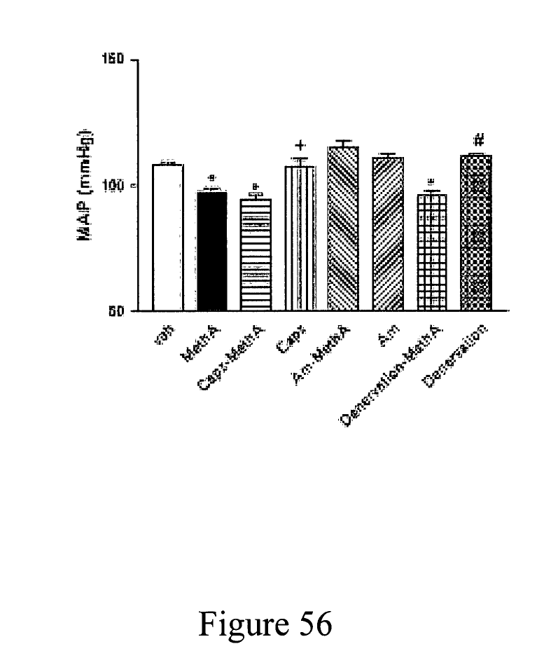

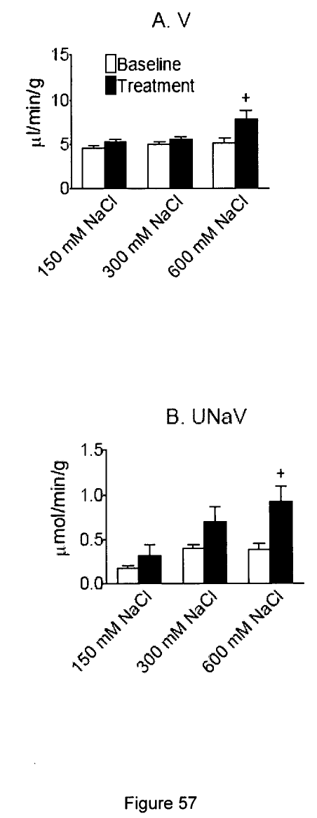

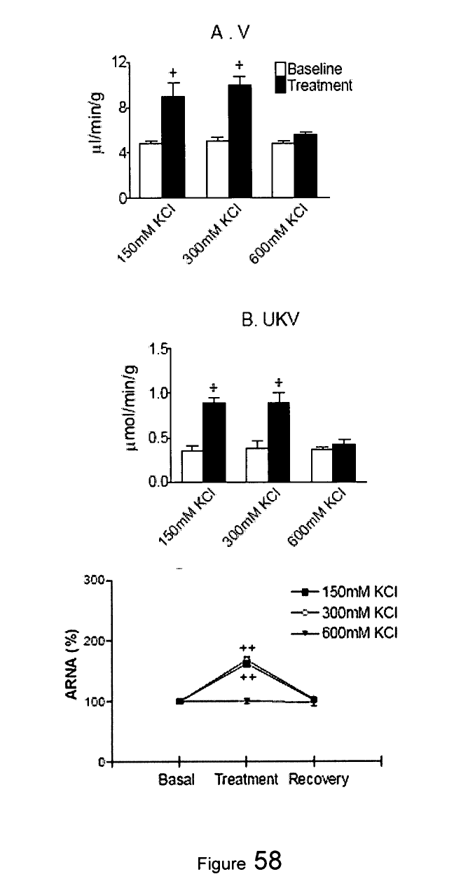

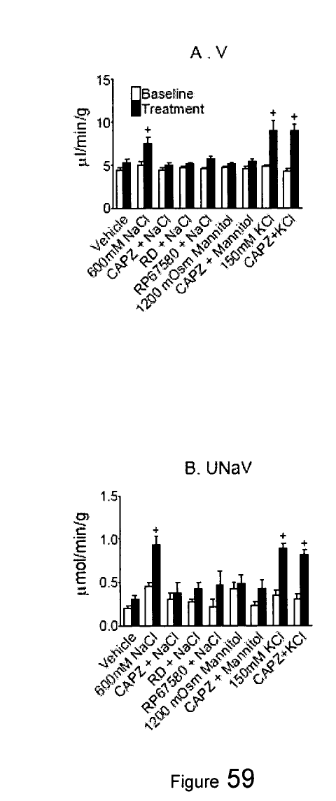

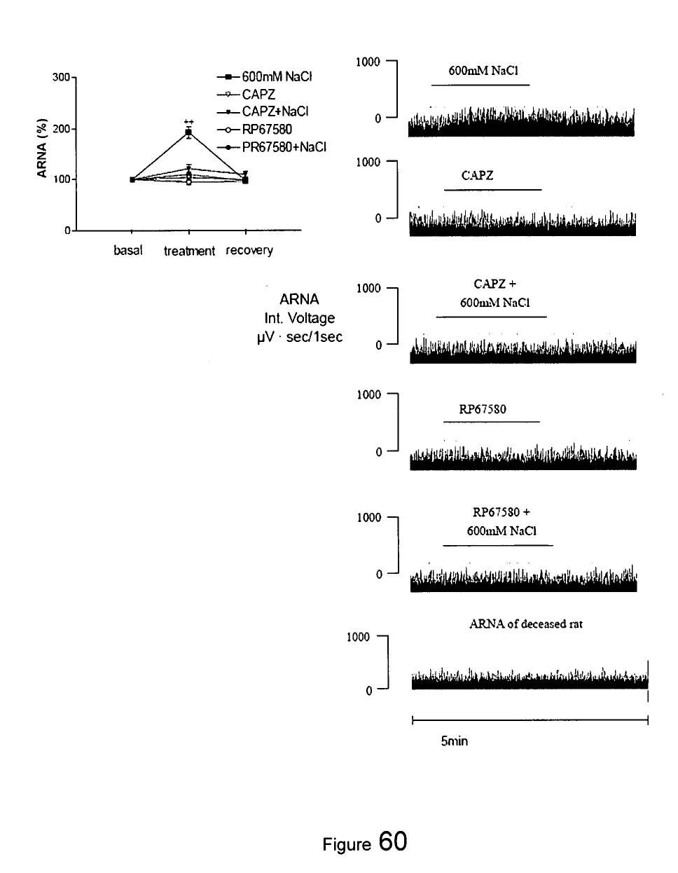

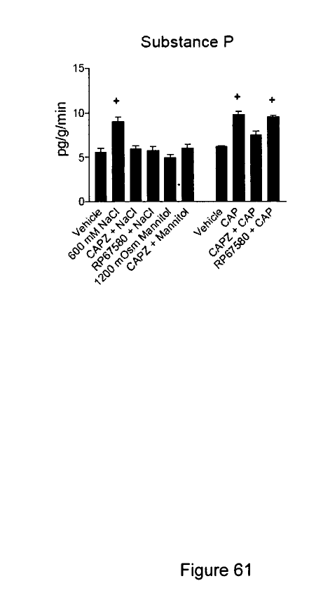

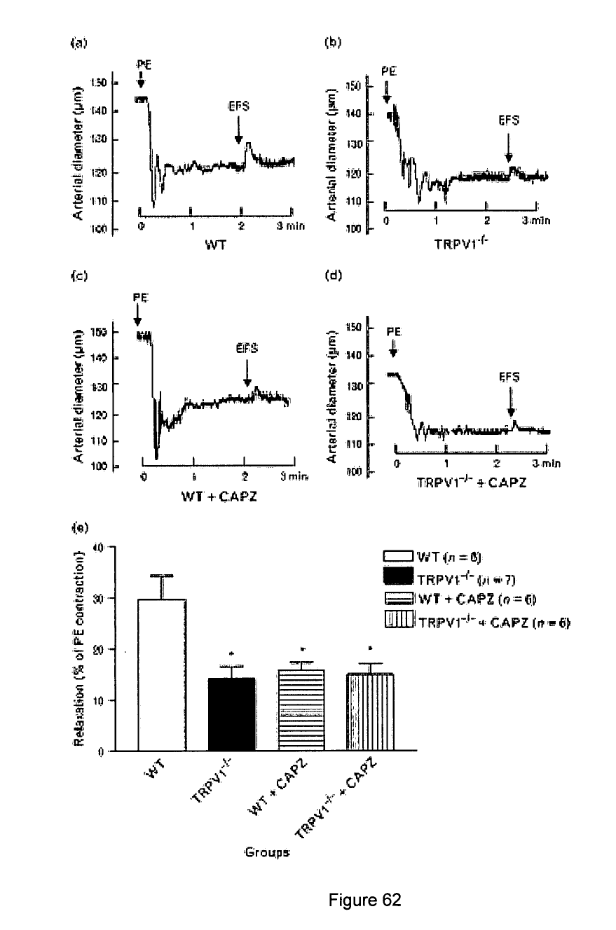

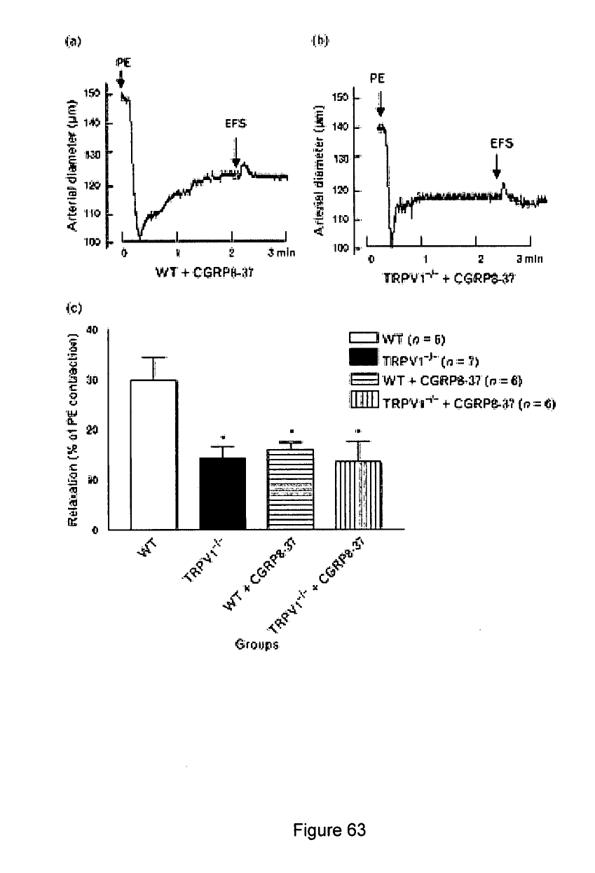

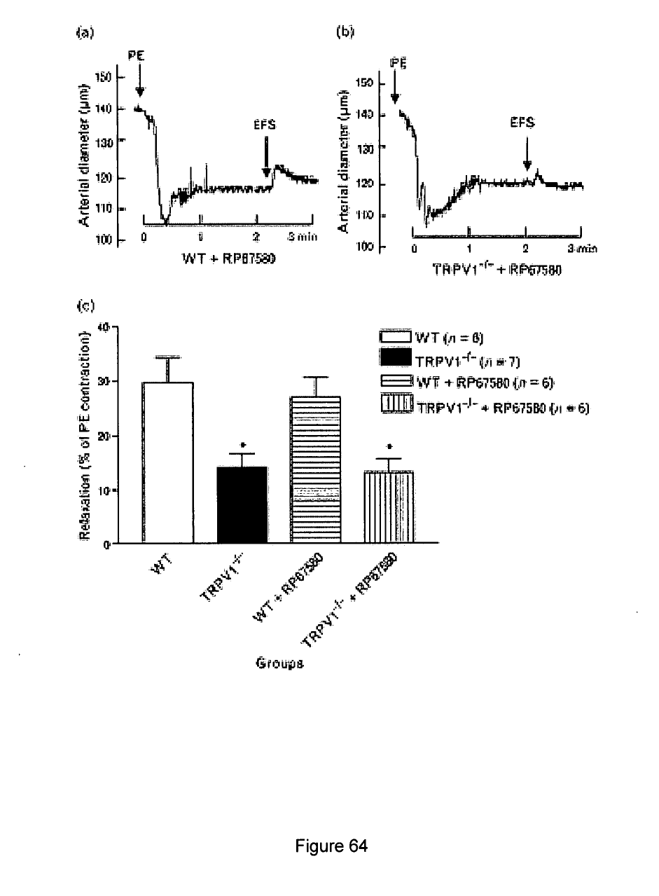

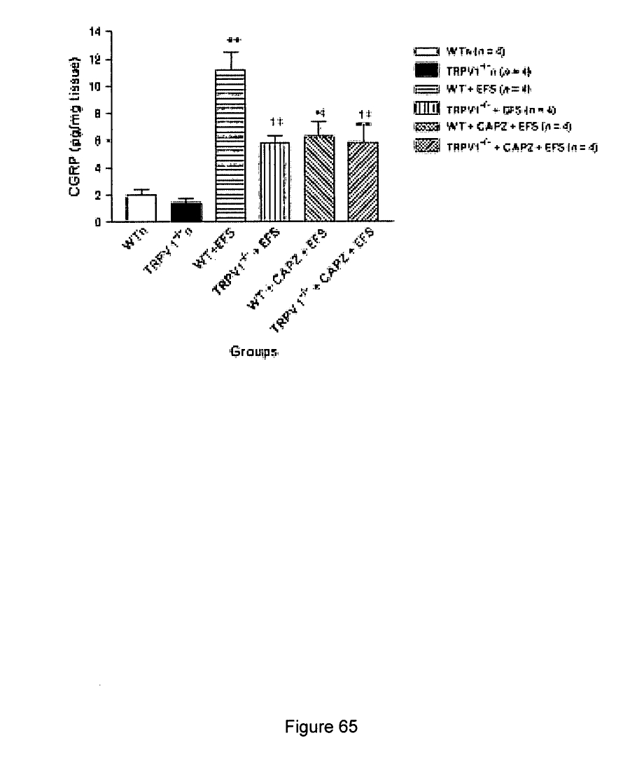

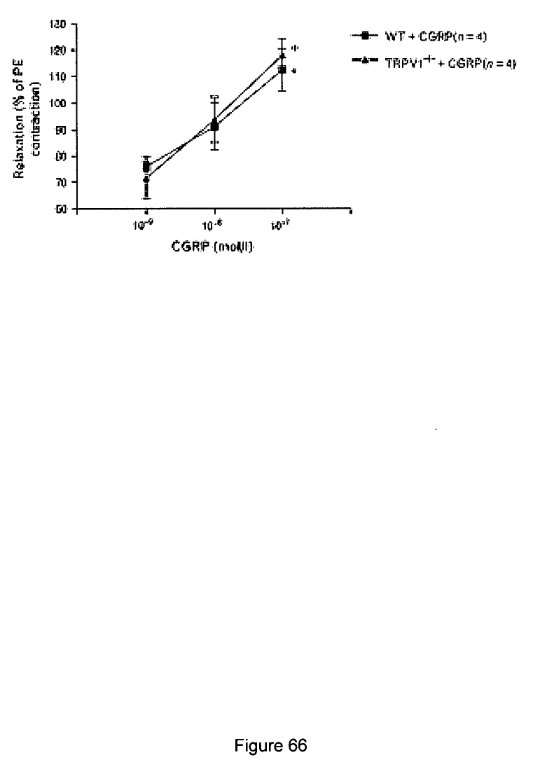

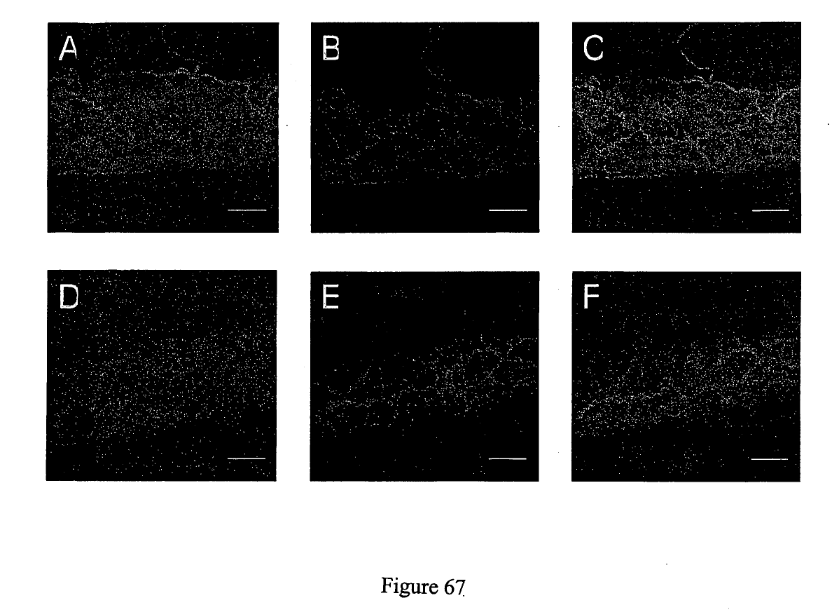

Creatinine clearance, a parameter representing glomerular filtration rate (GFR), is shown in FIG. 46. On day 14 after the dietary treatment, creatinine clearance (m1xmin-1 xg kidney wt-1) was significantly decreased in CAP−HS (0.25 0.03) compared with CON-NS (0.46 0.05), CON-HS (0.53 0.08), and CAP−NS rats (0.50 0.03) (P<0.05). Moreover, the O₂— levels in the renal cortex significantly correlated with creatinine clearance in CAP−HS rats (r=−0.76; P<0.001; FIG. 46B).

In summary, our data show that HS loading for 2 wk increases oxidative stress in the kidney of sensory-denervated rats, a finding that is supported by enhanced renal cortical and medullary O₂— production, further increased plasma and urinary 8-isoprostane levels, and increased activities and expression of NAD(P)H oxidase in these rats. These data suggest that sensory nerves play an important protective role against the production of O₂— in the kidney during HS intake.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, biochemistry, chemistry, molecular biology, or related fields are intended to be within the scope of the following claims.

We claim:

1. A method of treating a subject, comprising:
a) providing:
i) a subject, and
ii) a pharmaceutical composition comprising a therapeutic agent, wherein said therapeutic agent is selected from the group consisting of 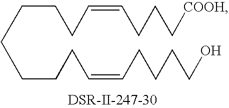
N-arachidonoyl dopamine (NADA), N-oleoyl-dopamine (OLDA), anandamide, Methanandamide (MethA), 20-hydroxyeicosatetraenoic acid (20-HETE), capsaicin (CAP), derivative or synthetic analog thereof, and a pharmaceutical carrier; and
b) administering said pharmaceutical composition to said subject.

2. The method of claim 1, wherein said pharmaceutical carrier comprises saline, a solvent, and a wetting agent.

3. The method of claim 1, wherein said solvent is selected from the group consisting of ethanol and dimethyl sulfoxide (DMSO).

4. The method of claim 1, wherein said wetting agent is selected from the group consisting of Tween 20 and Tween 80.

5. The method of claim 1, wherein said pharmaceutical carrier comprises a targeting agent for sensory nerves.

6. The method of claim 1, further comprising providing a side-effect reducing agent, wherein said side-effect reducing agent is co-administered to said patient.

7. The method of claim 1, wherein said side-effect reducing agent is a cannabinoid-1 receptor antagonist or a derivative or synthetic analog thereof.

8. A method of treating a patient demonstrating at least one symptom of salt sensitive hypertension, comprising:
a) providing:
i) a patient demonstrating one or more symptoms of salt induced hypertension, and
ii) a pharmaceutical composition comprising a therapeutic agent, wherein said therapeutic agent is selected from the group consisting of 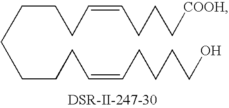
N-arachidonoyl dopamine (NADA), N-oleoyl-dopamine (OLDA), anandamide, Methanandamide (MethA), 20-hydroxyeicosatetraenoic acid (20-HETE), capsaicin (CAP), derivative or synthetic analog thereof, and a pharmaceutical carrier; and
b) administering said formulation to said patient under a condition such that one or more symptom of salt induced hypertension is reduced.

9. The method of claim 8, wherein said symptom of salt sensitive hypertension is selected from the group consisting of increased blood pressure, increased mean arteriole pressure, and decreased plasma renin levels.

10. The method of claim 8, wherein said patient is selected from a group consisting of a subject with a genetic predisposition for salt sensitivity, a population of subjects displaying a genetic predisposition for salt sensitivity, a subject with a nutritional imbalance for inducing a salt sensitivity, a subject with a hormonal imbalance for inducing a salt sensitivity, a subject exposed to an environmental factor for inducing a salt sensitivity, and a subject with high salt intake.

11. A method for drug screening comprising exposing a cell expressing a Transient Receptor Potential Vanilloid-1 (TRPV1) receptor to a test compound of interest and determining the activity of said cell in the presence and absence of said test compound.

12. A method for drug screening comprising:
a) providing:
i) a cell expressing a Transient Receptor Potential Vanilloid-1 (TRPV1) receptor; and
ii) a test compound;
b) exposing the cell to the test composition; and
c) determining the activity of said cell in the presence of said test composition.

13. The method of claim 12, wherein said cell is selected from the group consisting of a neuronal cell, a cardiac cell, a kidney cell, and an engineered cell.

14. The method of claim 12, wherein said cell is located within a tissue.

15. The method of claim 14, wherein said activity is selected from the group consisting of decreasing mean arteriole pressure, protecting against ischemia and reperfusion injury, increasing ventricular end-diastolic pressure, increasing coronary flow, decreasing ventricular peak positive dP/dt, decreasing plasma renin levels, increasing alpha calcitonin gene-related peptide release, and increasing substance P release.

16. The method of claim 12, wherein said cell is in vitro.

17. The method of claim 16, wherein said activity is selected from the group consisting of increasing alpha calcitonin gene-related peptide release, increasing substance P release, and increasing Ca release.

18. The method of claim 12, further providing a cell comprising an impaired Transient Receptor Potential Vanilloid-1 (TRPV1) receptor and d) determining the activity of said cell in the presence of said test composition, wherein said activity is decreased as compared to the activity of a cell expressing a Transient Receptor Potential Vanilloid-1 (TRPV1) receptor.

19. The method of claim 14, further providing an inhibitor and exposing said cell expressing Transient Receptor Potential Vanilloid-1 (TRPV1) receptor to said inhibitor and determining the activity of said cell, wherein said activity is decreased.

20. The method of claim 19, wherein said method further provides a cell expressing a cannabinoid-1 receptor, and a step for exposing the cell to the test compound and determining cannabinoid-1 receptor activity, wherein said exposing reduces activation of said cannabinoid-1 receptor.

21. The method of claim 19, wherein said cell is in a whole organ selected from the group consisting of a heart and a kidney.
